(19) United States
(12) Patent Application Publication
(10) Pub. No.: US 2022/0026434 A1
(43) Pub. Date: Jan. 27, 2022

(54) ADVANCED METHODS FOR AUTOMATED HIGH-PERFORMANCE IDENTIFICATION OF CARBOHYDRATES AND CARBOHYDRATE MIXTURE COMPOSITION PATTERNS AND SYSTEMS THEREFORE AS WELL AS METHODS FOR CALIBRATION OF MULTI WAVELENGTH FLUORESCENCE DETECTION SYSTEMS THEREFORE, BASED ON NEW FLUORESCENT DYES

(71) Applicant: MAX-PLANCK-GESELLSCHAFT ZUR FÖRDERUNG DER WISSENSCHAFTEN E.V, München (DE)

(72) Inventors: Erdmann RAPP, Magdeburg (DE); René HENNIG, Magdeburg (DE); Udo REICHL, Magdeburg (DE); Stefan HELL, Göttingen (DE); Vladimir BELOV, Göttingen (DE); Matthias BISCHOFF, Dortmund (DE); Dirk MEINEKE, Köln (DE); Laura THOMAS, Lüneburg (DE); Kirill KOLMAKOV, Magdeburg (DE); Gyuzel MITRONOVA, Göttingen (DE); Elizaveta SAVICHEVA, St. Petersburg (RU)

(21) Appl. No.: 17/424,265

(22) PCT Filed: Jan. 21, 2019
(86) PCT No.: PCT/EP2019/051351

§ 371 Date: Jul. 20, 2021

(51) Int. Cl. G01N 33/58; G01N 1/30; G01N 21/64; G01N 30/86; G01N 33/52

(52) U.S. Cl. CPC G01N 33/582; G01N 1/30; G01N 21/64; G01N 2021/6441; G01N 33/52; G01N 2001/302; G01N 30/86

(57) ABSTRACT

The present invention relates to improved (simplified/easier, more robust and more reproducible) methods for identification of carbohydrates compositions, e.g. out of complex carbohydrate mixtures, as well as the determination of carbohydrate mixture composition patterns (e.g.: of glycosylation patterns) based on advanced internal standards to determine precise and highly reproducible migration and retention time indices using novel fluorescent dyes in combination with high performance separation technologies, like capillary (gel) electrophoresis (C(G)E) or (ultra)high performance liquid chromatography (U)HPLC with a highly sensitive detection like (laser induced) fluorescence detection. In a first aspect, the present invention relates to methods for an automated determination and/or identification of carbohydrates and/or carbohydrate mixture composition pattern profiling as well as a method for an automated carbohydrate mixture composition pattern profiling based on the use of at least a first and second fluorescent label for labelling the migration/retention time alignment standard and sample or different samples, respectively, whereby the at least one of that fluorescent dye is a compound as defined herein. Moreover, the present invention relates to a method for calibration of multi wavelength fluorescence detection systems as well as calibration systems or calibration standards and new compounds suitable for calibration are described. The present invention relates further to a kit or system for determining or identifying carbohydrate mixture composition patterns as well as a kit or system for determining and/or identifying carbohydrate mixture composition pattern. Further, a carbohydrate dye conjugate comprising the dye as defined herein for use in a method according to the present invention is provided. The dyes employed for forming the carbohydrate dye conjugate have formula A or B below:

The present invention relates to improved (namely, simplified/easier, more robust and more reproducible) methods for identification of carbohydrates compositions, e.g. out of complex carbohydrate mixtures, as well as the determination of carbohydrate mixture composition patterns (e.g.: of glycosylation patterns) based on advanced internal standards to determine precise and highly reproducible migration and retention time indices using novel fluorescent dyes in combination with high performance separation technologies, like capillary (gel) electrophoresis (C(G)E) or (ultra)high performance liquid chromatography (U)HPLC with a highly sensitive detection like (laser induced) fluorescence detection.

In a first aspect, the present invention relates to methods for an automated determination and/or identification of carbohydrates and/or carbohydrate mixture composition pattern profiling as well as a method for an automated carbohydrate mixture composition pattern profiling based on the use of at least a first and second fluorescent label for labelling the migration/retention time alignment standard and sample or different samples, respectively, whereby the at least one of that fluorescent dye is a compound as defined herein.

Moreover, the present invention relates to a method for calibration of multi wavelength fluorescence detection systems as well as calibration systems or calibration standards and new compounds suitable for calibration are described.

The present invention relates further to a kit or system for determining or identifying carbohydrate mixture composition patterns as well as a kit or system for determining and/or identifying carbohydrate mixture composition pattern. Further, a carbohydrate dye conjugate comprising the dye as defined herein for use in a method according to the present invention is provided.

### PRIOR ART

The importance of glycosylation in many biological processes is commonly accepted, a discussion is in the literature over decades. Glycosylation is a common and highly diverse post-translational modification of proteins in eukaryotic cells. Various cellular processes have been described, involving carbohydrates on the protein surface. The importance of glycans in protein stability, protein folding and protease resistance have been demonstrated in the literature. In addition, the role of glycans in cellular signaling, regulation and developmental processes has been demonstrated in the art.

Carbohydrate(s) is the umbrella term for monosaccharide(s), like xylose arabinose, glucose, galactose, mannose, fructose, fucose, N-acetylglucoseamine, sialic acids; (homo or hetero) disaccharide(s), like lactose, sucrose, maltose, cellobiose; (homo or hetero) oligosaccharide(s), like glycans (e.g. N- and O-glycans), galacto-oligosaccharides (GOS), fructooligosaccharides (FOS), milk oligosaccharides (MOS) or even the glycomoiety of glycolipids; and polysaccharide(s), like amylose, amylopektin, cellulose, glycogen, glycosaminoglycan, or chitin. Oligo- and polysaccharides can either be linear or (multiple) branched.

Glycoconjugates are compounds in which a carbohydrate (the glycone) is linked to a non-carbohydrate moiety (the aglycone). Typically, the aglycone is either a protein or a lipid, thus, the glycoconjugate are termed glycoprotein or glycolipid respectively. In a more general sense, glycoconjugate means a carbohydrate covalently linked to any other chemical entity including protein, peptide, lipid or even saccharide.

Glycoconjugates represent the structurally and functionally most diverse molecules in nature. Starting from simple glycoconjugates composed of a nucleotide and a single sugar moiety to extraordinary complex and multiple glycosylated proteins. The most common carbohydrate moieties in glycoconjugates are concentrated on a few monosaccharides, including N-acetylglucosamine, N-acetylgalactosamine, mannose, galactose, fucose, glucose as well as xylose and sialic acids and modifications thereof including modifications being phosphorylated or sulfated, the structural diversity is possibly much larger than that of proteins or DNA.

The reasons for this diversity are the presence of the anomers and the ability of monosaccharides to branch and to build different, glycosylic linkages. Accordingly, an oligosaccharide with the relatively small chain length may have an enormous number of structural isomers. In contrast to protein biosynthesis, which is based on RNA as a template, the information flow from the genome to the glycome is complex and, in addition, not a template driven process. Co- and post-translational modification of e.g. proteins in glycan biosynthesis is based on enzymatic reactions. Due to the glycan biosynthesis a drastic increase of complexity and structural diversity of the glycans is present. Of note, the term “glycan” is used synonymously to the term glycone, both referring to the carbohydrate portion of the glycoconjugate.

Further, the terms glycan, oligosaccharides and polysaccharides are used synonymously referring to “compounds having a moiety of a (medium or large) number of monosaccharides linked glycosidically”. In proteins, the oligosaccharides are mainly attached to the protein backbone, either by N-(via Asn) or O-(via Ser or Thr) glycosidic bonds, whereas N-glycosylation represents the more common type found in glycoproteins. Variations in glycosylation site occupancy (macro-heterogeneity), as well as variations in these complex sugar residues attached to one glycosylation site (micro-heterogeneity) results in a set of different protein glycoforms. These have different physical and biochemical properties which results in additional functional diversity of the glycoproteins. For example, in manufacturing of therapeutic proteins in mammalian cell cultures, macro- and micro heterogeneity were shown to affect properties of the proteins. For instance, the relevance of the glycosylation profile for the therapeutic profile of monoclonal antibody is well documented. Of note, the glycan structures, in particular, the N-glycan structures are also depending on various factors during the production process, like substrates levels and other cultural conditions. Thus, the glycoprotein manufacturing does not only depend on the glycosylation machinery of the host cell but also on external parameters, like cultural conditions and the extracellular environment. Further parameters effecting the glycosylation in culture production include temperature, pH, aeration, supply of substrates or accumulation of byproducts, such as ammonia and lactate. For example, in the pharmaceutical field the glycosylation profiles are of particular interest since due to regulatory reasons, the glycosylation profile of drugs has to be determined.

Also in food and pharmaceutical industry the beneficial effects of different types of glycoconjugates, namely, having nutritional and/or biological effects are gaining increasing interest. Today, complex soluble but also oligomeric and/or polymeric carbohydrate mixtures, obtained synthetically or from natural sources, like plants or human or animal milk are used as nutrition additives or in pharmaceuticals. The occurrence of sialic acids or sialic acid derivatives and the occurrence of monosaccharides having a phosphate, sulphate or carboxyl group within those complex natural carbohydrates is even increasing their complexity. Because of this complexity, those prebiotic oligo- or polysaccharides, like neutral or acidic galacto-oligosaccharides, long chain fructo-oligosaccharides or (human) milk oligosaccharides ((H)MOS), which can have nutritional and/or biological effects, are gaining increasing interest for food and pharmaceutic industry.

In order to elucidate the structural features of the glycome, which means the complete set of free carbohydrates and glycoconjugates in cells produced under specific conditions and to understand its functions and its counterplay with DNA and protein machinery, rapid, robust and high resolution by analytical techniques must be available.

A wide range of strategies and analytical techniques for analyzing glycoconjugates including glycoproteins, glycopeptides and released N-glycans or O-glycans have been established. For example, complex samples containing a variety of different oligosaccharides can be separated by chromatographic or electrokinetic techniques. These techniques include chromatographic techniques like size exclusion chromatography (SEC), hydrophilic interaction chromatography (HILIC), reversed phase liquid chromatography (RPLC) and reversed phase ion pairing chromatography (RPIPC), as well as porous graphitized carbon chromatography (PGC). Further, structural data of complex molecules including carbohydrates derived from glycoconjugates are either analyzed by mass-spectrometry (MS) or nuclear magnetic resonance spectroscopy (NMR) which are generally laborious and time-consuming techniques regarding sample preparation and data interpretation. For example, a combination of several techniques is often applied like combination of liquid chromatography (LC) with NMR or MS or combination of capillary electrophoresis (CE) with MS or NMR. Typically, a glycosylation pattern is obtained, also identified as a carbohydrate mixture composition pattern identifying characteristic properties of said glycan, such as retention or migration times. By comparing data obtained from unknown samples with determined parameters, the rapid screening and evaluation of unknown samples can be performed.

Each of these techniques has advantages as well as drawbacks. Choosing one, respectively a set of these methods for a given problem can become a time- and labor-intensive task. For example, NMR provides detailed structural information, but is a relatively insensitive method (nmol), which cannot be used as a high-throughput method. Using MS is more sensitive (fmol) than NMR. However, quantification can be difficult and only unspecific structural information can be obtained without addressing linkages of monomeric sugar compounds. Both techniques require extensive sample preparation and also fractionation of complex glycan mixtures before analysis to allow evaluation of the corresponding spectra. Furthermore, a staff of highly skilled scientists is required to ensure that these two techniques can be performed properly.

Easier, cheaper and thus more common are electrokinetic and chromatographic separation-based analytical methods. Most common and adulterated are the chromatographic glycoanalytical techniques, like hydrophilic interaction chromatography with fluorescence detection (HILIC-FLR), reversed phase liquid chromatography with fluorescence detection (RPLC-FLR). They can be operated as high performance or as ultra-high-performance liquid chromatography (HPLC or UHPLC), but up to now only with an external standard (i.e.: not together with the sample within the same run and separation column, like with an internal standard) for retention-time alignment, and therefore only with limited (long-term) reproducibility (Kobata A, et al., Methods Enzymology 1987, 138, 84-94. Tomiya N, et al., Analytical Biochemistry 1988, 171, 73-90. Guile G R, et al., Analytical Biochemistry 1996, 240, 210-226.

Although separation techniques based on the capillary electrophoresis principle, like capillary gel electrophoresis were considered for complex carbohydrate separation in the art before, e.g. Callewaert, N. et al., Glycobiology 2001, 11, 275-281, WO 01/92890, Callewaert, N. et al., Nat. Med. 2004, 10, 429-434, Hennig R, et al., Biochimica et Biophysica Acta—General Subjects 2016, 1860, 1728-1738, Ruhaak L R, et al., Journal of Proteome Research 2010, 9, 6655-6664, EP2112506 A1 there is still an ongoing need for a reliable and fast system allowing automated high throughput carbohydrate analysis.

Examples of the electrokinetic separation techniques are capillary electrophoresis (CE) and capillary gel electrophoresis (CGE). These techniques allow high resolution, fast separation and also quantification. For example, multiplex capillary gel electrophoresis with laser induced fluorescence detection (xCGE-LIF) has shown to be an especially powerful tool for glycoanalysis. An advantage of the multiplex capillary array setup is the potential for very high throughput analysis due to parallelization of separation. Another reason for using xCGE-LIF is the very high sensitivity due to LIF detection. CGE is defined as “a special case of capillary sieving electrophoresis wherein the capillary is filled with a cross-linked gel (polymer)”.

The electrophoretic mobility of a compound depends on the mass to charge ratio, and when employing e.g. CGE due to the gel sieving effect, it depends additionally from the molecular shape. Commonly, native carbohydrates cannot be separated by their mass to charge ratio, because most of them are electroneutral except the ones that contain charge residues, like sialic acid, glucuronic acids, sulphated or phosphorylated moieties. However, a problem of CE the (long-term) reproducibility of the migration times, e.g. in CGE due to ageing of the gel present in the capillaries. Therefore, up to now, its usability has some limitations, even when using internal standards for migration time alignment (like a DNA basepair (bp) ladder with a fluorescent tag emitting at a different wavelength than the dye (e.g. APTS) of the carbohydrate sample), as despite comparable mass-to-charge ration (m/z), m and z both are very different for the bp alignment standard and the carbohydrate sample see EP2112506 A1. Therefore, the matrix (e.g. content and composition of salts, solvents, gel, etc.) but also temperature and time (which are also causing changes of the matrix, e.g. due to gel-ageing) are decreasing reproducibility and therefore usability.

Since Sanger discovered the chain termination method for the sequencing of DNA in 1977, big advances were made to increase the sequencing throughput. The first improvement was made in the mid-80s by replacing the radiolabeling of DNA fragments by the labeling with fluorescent dyes. By labeling each DNA base with an individual fluorescent dye (comprising distinct excitation and emission wavelengths), all four reaction mixture could be loaded into one lane of a slab-gel and simultaneously analyzed. A laser scanning system with an optical filter, enabled the wavelength resolve detection of the fluorescent emission from all four dyes (respectively all DNA bases) separately. The conversion into a digital signal pave the way to the development of the automated DNA sequences, like the ABI PRISM 377. Genetic Analyzer.

In conventional slab-gel electrophoresis systems multiple samples are separated in a thin gel with many individual lanes. Unfortunately, it was difficult increase throughput, as the separation speed was limited by the field strength which could not be increased as it generates heat in the gel. Furthermore, the detection speed was limited to one up to several seconds per data point.

To overcome this issue capillary electrophoreses (CE) systems were developed with several parallel capillary tubes (capillary array) with a diameter of only 10-50 μm. Due to its big surface per volume a better heat transfer was achieved, allowing at higher field strength and a lot faster separation. Optimized optics inside these multi-capillary CE systems, with a laser beam aligned transversely to the parallel capillaries, allowed a simultaneously excitation of all fluorescent labeled analytes inside all capillaries. These laser-induced fluorescence (LIF) detection offered the lowest limits of detection. During the detection the emitted fluorescence is filtered with a virtual filter set (observation windows), followed by the capturing of the fluorescence signals from the defined individual channels (multi-wavelength detection) by a CCD camera.

FIG. 32: Detection mode of multi-capillary CE systems with multi-wavelength detection.

Since fluorescent dye emission spectra are always rather broad and overlapping (as shown in Scheme 1) virtual filters need to be calibrated. Thereby the intended is not to collect the emission at its maximum, rather than to minimize overlap of the emission profiles on the CCD array. However, the spectral overlap still occurs to some extent, and a certain cross-talk is always present, as sown in Scheme 1 for the middle fluorescent dye.

For DNA sequencing each of the four nucleotides is labeled with one fluorescent dye. During the sequencing always the most prominent peak in a color channel is picked and defines the nucleotide. The problem of spectral cross-talk is not much important for DNA sequencing, as the smaller cross-talk signal from the neighbor dye channel is not considered.

For analysis of oligosaccharide by multiple/multiplexed CE (xCE) systems completely other demands are to be met. In general an unknown sample labeled with one fluorescent dye is co-injected and co-separated with an alignment standard labeled with another fluorescent dye. This internal standard is subsequent used for the alignment of the migration time of the unknown sample. By this alignment an automated determination and/or identification of the sample composition is possible.

For a proper analysis the absence of spectral cross-talk between the two dye channels (unknown sample vs. alignment standard) is necessary. For instance the electropherogram of an unknown sample (complex oligosaccharide mixture) contains peaks with intensities varying in several orders of magnitude. Signals “leaking” from the channel of the alignment standard would produce additional peaks, change the composition of the unknown sample, and hence burden the analysis. In order to eliminate cross-talk between dye channels, it is crucial to re-calibrate the multiplexed CE system.

Native carbohydrates are poorly detectable by spectroscopic methods. Only UV light at wavelengths below 200 nm permits detection. To overcome this drawback, released N-glycans are labeled with a fluorescent tag before (chromatographic or electrokinetic) separation, to make them well detectable for e.g. UV, VIS, FLR and LIF detectors.

FIG. 1 shows the main steps of separation based glycananalysis. The procedure can be divided into the following steps: sample preparation, chromatographic or electrokinetic separation with fluorescent detection and data evaluation. Labelling of glycans and detection of labelled products are described in the art. The principle reaction mechanism of reductive amination used for fluorescent labeling of carbohydrates is shown in Scheme 2.

Scheme 2 below shows the principal reaction sequence of the reductive amination of carbohydrates (cf., N. Volpi, Capillary electrophoresis of carbohydrates. From monosaccharides to complex polysaccharides, Humana Press, New York, 2011, pp. 1-51).

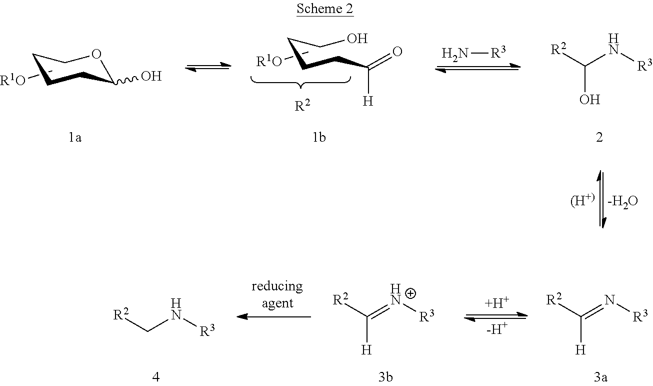

The first step of the reductive amination involves a nucleophilic addition reaction where the lone electron pair of the amine nitrogen attacks the electrophilic aldehyde carbon atom of the carbohydrate residue in its open-chain form (1b). The acid-catalyzed elimination of water from intermediate 2 gives an imine (3a). Since the imine formation is reversible, the imine has to be converted into a secondary amine (4) via irreversible acid-catalyzed reduction with a hydride source (reducing agent in Scheme 2). The nature of the reducing agent is important, because only iminium ions 3b need to be reduced, while carbohydrates R²CHO (1b) have to remain unreactive towards the reduction (they react only with amines R³NH₂ which represent fluorescent tags).

The reaction sequence depicted in Scheme 2 is based on the availability and sufficient reactivity of special reducing agents (boranes) which do not react with aldehydes (or reduce them very slowly), but under acidic conditions readily reduce iminium ions (3b). Weak or medium strong acids such as acetic (pKa=4.76), malonic (pK1a=2.83) or citric acid (pK1a=3.13) are frequently used at pH=3-6 to achieve an irreversible and rapid reduction (K. R. Anumula, Anal. Biochem. 2006, 350, 1-23). Therefore, the applied amine (R³NH2) has to be a weak base (because only the non-protonated amine can react with aldehyde 1b in Scheme 2). In proteins, the aliphatic amino groups of lysine, nucleophilic nitrogen atoms in histidine and arginine residues are protonated at pH=3-6 and do not react with carbohydrates according to Scheme 2. Therefore, only aromatic amines with rather low pKa values of 3-5 (these are values for the conjugated acids) are required and widely used as analytical reagents for reductive amination of natural glycans. Shown below are 3 commercially available aromatic amines applicable for labeling of glycans via reductive amination, chromatographic or electrokinetic separation of conjugates and sensitive detection by fluorescence.

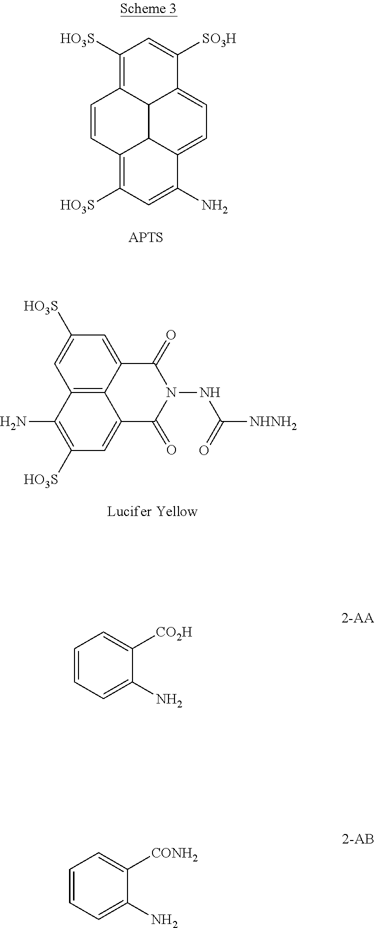 [SMILES: NC1=CC(S(=O)(=O)O)=C2C=CC3=C(S(=O)(=O)O)C=C(S(=O)(=O)O)C4=CC=C1C2C43 ; NNC(=O)NN1C(=O)c2cc(S(=O)(=O)O)cc3c(N)c(S(=O)(=O)O)cc(c23)C1=O ; Nc1ccccc1C(=O)O ; NC(=O)c1ccccc1N]

3-Aminopyrene-1,6,8-trisulfonic acid (APTS), 2-aminobenzamide (2-AB) and 2-Aminobenzoic acid (2-AA) are currently the most widely used reagent for carbohydrate labeling for CE (APTS) and LC (2-AB and 2-AA) bases analytic. Especially, APTS with its three strong acidic residues (sulfonic acid groups) introduce three negative charges in a very wide pH range (at pH >2), allowing a flexible and robust analysis.

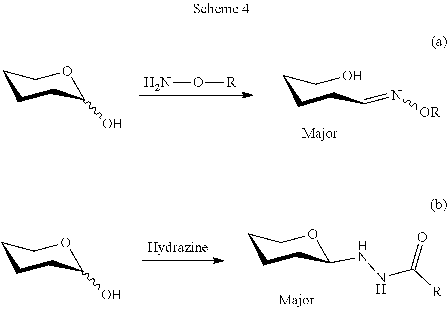

Alkyloxyamino (Scheme 4a) and hydrazide (Scheme 4b) groups also provide a convenient, chemo-selective method for labeling of carbohydrates. Hydrazide groups in reaction with the reducing end of free carbohydrates form a product in predominantly cyclic β-anomeric form see Scheme 4b). Reaction conditions range from acidic, over neutral to basic pH at elevated temperatures. A typical hydrazide labeling reaction of e.g. Lucifer Yellow (see Scheme 3) could be performed at 70° C. for 1 h at pH 7.

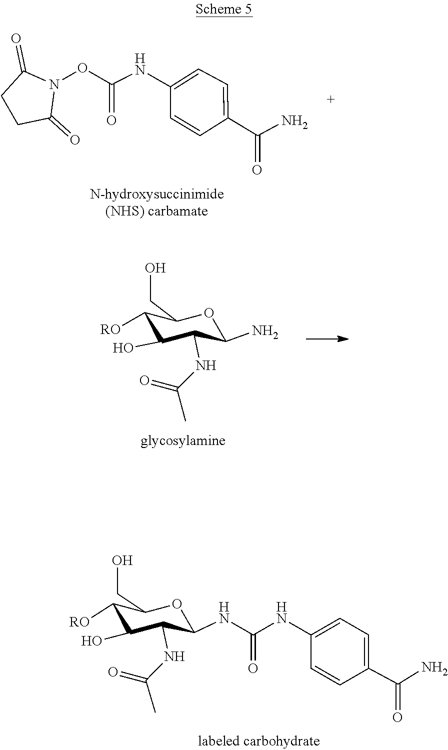

Furthermore, a reactive carbamate chemistry can be used for the labeling of carbohydrates, as shown in Scheme 5. For this labeling reaction the carbohydrate is needed in his glycosylamine form (released carbohydrate form a glycoconjugate e.g. N-glycans after enzymatic release by PNGase F). This reaction is rather unspecific, because the reactive carbamate can react with other available amines of e.g. proteins (amino acid lysine). A typical reaction of N-hydroxysuccinimide (NHS) carbonate with a glycosylamine takes place at room temperature just in minutes.

As the reductive amination of carbohydrate is really specific and complete, this reaction is currently the most widely used carbohydrate labeling procedure.

After facultative purification (to remove proteins, excess electrolytes, excess dye, labeling reagents, etc.), the labeled sample is injected into the chromatographic column, respectively the electrokinetic capillary, and the separation is carried out (see FIG. 1). Due to their different properties (like hydrophobicity, mass/charge, shape, etc.) the different carbohydrates reach the detector according to their characteristic retention, respectively, migration times (see FIG. 2-22).

When the labeled carbohydrates reach the fluorescence detector, the covalently linked fluorescent dyes are excited and the emission signal is detected.

Today, analysis of glycans is performed on commercial (U)HPLC systems with a fluorescence detector after labeling them e.g. with 2-AB or 2-AA (see Scheme 3), but “real” high throughput analysis of labeled glycans is can only be performed on commercial multiplex CGE-systems. These xCGE-LIF instruments contain a multiplexed capillary gel electrophoresis unit for the separation of charged analytes (e.g., APTS-labeled glycans), a laser and a fluorescence detector.

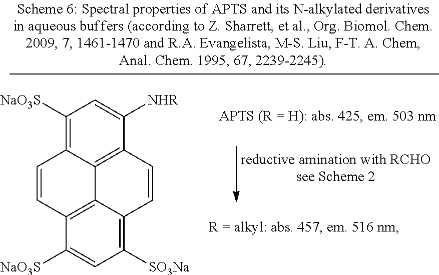

Other dyes than APTS may be used as fluorescent tags for separation-based analysis of carbohydrates and their derivatives (e.g., dyes 2-AB, 2-AA and LuciferYellow, see Scheme 3 and the review by N. V. Shilova and N. V. Bovin, Russ. J. Bioorg. Chem. 2003, 29 (4), 339-355. Further examples are acridone dyes, described in WO 2002/099424 A3 and WO 2009/112791 A2, but not 7-aminoacridone-2-sulfonamides. WO 2012/027717 A1 describes systems comprising functionally substituted 1,6,8-trisulfonamido-3-aminopyrenes (APTS derivatives), an analyte-reactive group, a cleavable anchor as well as a porous solid phase. WO 2010/116142 A2 describes a large variety of fluorophores and fluorescent sensors compounds which also encompass aminopyrene-based dyes. However, none of these dyes has been shown or suggested to have superior spectral and electrophoretic properties, in particular as conjugates with carbohydrates, in comparison with APTS.

Separation techniques and analysis of carbohydrates and glycosylation pattern profiling is described in the art. For example, Callewaert N et al, Glycobiology 2001, 11, 275-281, WO 01/92890, Callewaert N. et al, Nat. Med., 2004, 10, 429-439 or Khandurina et al, Electrophoresis, 2004, 25, 3122-2127 identify methods for carbohydrate analysis. Domann et al., Practical Proteomics, 2007, 7, 70-76 identify 2DHPLC profiling, mass-spectrometry and lectin affinity chromatography.

Further developments are described in EP 2112506 A1 and US 2009/0288951 A1 by the present inventors. The technique described therein has been applied successfully.

However, a main drawback for evaluating glycan profiles is the limited availability of suitable dyes. Namely, none of the dyes known so far are suggested to have superior spectral or electrophoretic properties, in particular as conjugates with carbohydrates, but the present standard is the use of APTS.

Hence, there is a need for fluorescent dyes with improved properties, such as higher electrophoretic mobility and/or higher brightness, compared to APTS. These properties are highly demanded for fluorescent tags for carbohydrate analysis based on electrokinetic, respectively, chromatographic separations separated with fluorescence detection, allowing superior performance. In addition, there is a need for fluorescent dyes which can be used in combination with known dyes including APTS, thus, allowing detection of two different colors within the same run and thus an internal alignment of the migration, respectively, retention times.

### DESCRIPTION OF THE PRESENT INVENTION

The goal of the present invention is to provide new methods for determining and/or identifying carbohydrates and/or carbohydrate mixture composition pattern profiling based on retention/migration time alignment to internal standard(s) using at least two different fluorescent dyes allowing a highly reproducible electrokinetic/chromatographic separation with subsequent fluorescent detection or laser induced fluorescence detection. The labelling of a carbohydrate sample and a carbohydrate standard with at least two suitable fluorescent dyes, emitting at different wavelengths, is indispensable for such an internal migration/retention time alignment, enabling high long-term reproducibility and matrix/sample independency as discussed below.

In a first aspect, a method for an automated determination and/or identification of carbohydrates and/or carbohydrate mixture composition pattern profiling comprising the steps of:

a) obtaining a sample containing at least one carbohydrate;
b) labelling said carbohydrate(s) with a first fluorescent label;
c) providing a standard of known composition labelled with a second fluorescent label;
d) determining the migration/retention time(s) of said carbohydrate(s) and the standard of known composition using electrokinetic/chromatographic separation techniques combined with fluorescence or laser induced fluorescence detection;
e) aligning the migration/retention time(s) to migration/retention time indice(s) based on given standard migration/retention time indice(s) of the standard;
f) comparing these migration/retention time indice(s) of the carbohydrate(s) with standard migration/retention time indice(s) from a database;
g) identifying or determining the carbohydrate(s) and/or the carbohydrate mixture composition pattern,
wherein the standard composition is added to the sample containing the unknown carbohydrate and/or carbohydrate mixture composition, the first fluorescent label and the second fluorescent label are different and wherein the first fluorescent label or the second fluorescent label is a fluorescent dye having multiple ionizable and/or negatively charged groups which is selected from the group consisting of compounds of the following general Formulae A and B:

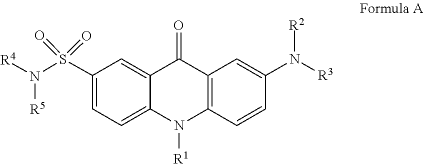

wherein

R¹, R², R³, R⁴, R⁵ are independent from each other and may represent:

H, CH₃, C₂H₅, a straight or branched C₃-C₁₂, preferably C₃-C₆, alkyl or perfluoroalkyl group, a phosphorylated alkyl group (CH₂)ₘP(O)(OH)₂, where m=1-12, preferably m=2-6, with a straight or branched alkyl chain, (CH₂)ₙCOOH, where n=1-12, preferably n=1-5, or (CH₂)ₙCOOR⁶, where n=1-12, preferably n=1-5, and R⁶ may be alkyl, in particular C₁-C₆ alkyl, CH₂CN, benzyl, fluorene-9-yl, polyhalogenoalkyl, polyhalogenophenyl, e.g. tetra- or pentafluorophenyl, pentachlorophenyl, 2- and 4-nitrophenyl, N-succinimidyl, sulfo-N-succinimidyl, 1-oxybenzotriazol or other potentially nucleophile-reactive leaving groups, alkyl sulfonate ((CH₂)ₙSO₃H) or alkyl sulfate ((CH₂)ₙOSO₃H) where n=1-12, preferably n=1-5, and the alkyl chain in any (CH₂)ₙ may be straight or branched;

a hydroxyalkyl group (CH₂)ₘOH orthioalkyl group (CH₂)ₘSH, where m=1-12, preferably m=2-6, with a straight or branched alkyl chain, a phosphorylated hydroxyalkyl group (CH₂)ₘOP(O)(OH)₂, where m=1-12, preferably m=2-6, with a straight or branched alkyl chain; one of R¹ or R² groups may be a carbonate or carbamate derivative (CH₂)ₘOCOOR⁷ or COOR⁷, where m=1-12 and R⁷=methyl, ethyl, tertbutyl, benzyl, fluoren-9-yl, CH₂CN, N-succinimidyl, sulfo-N-succinimidyl, 1-oxybenzotriazolyl, phenyl, substituted phenyl group, e.g., 2- or 4-nitrophenyl, pentachlorophenyl, penta-fluorophenyl, 2,3,5,6-tetrafluorophenyl, 2-pyridyl, 4-pyridyl, pyrimid-4-yl;

(CH₂)ₘNR^{a}R^{b}, where m=1-12, preferably 2-6, with a straight or branched alkyl chain; R^{a}, R^{b} are independent from each other and represent hydrogen and/or C₁-C₄ alkyl groups, a hydroxyalkyl group (CH₂)ₘOH, where m=2-6, with a straight or branched alkyl chain, a phosphorylated hydroxyalkyl group (CH₂)ₘOP(O)(OH)₂, where m=1-12, preferably 2-6, with a straight or branched alkyl chain; an alkyl azide (CH₂)ₘN₃, where m=1-12, preferably 2-6, with a straight or branched alkyl chain;

R¹, R², R³, R⁴, R⁵ may contain a terminal alkyloxyamino group (CH₂)ₘONH₂, where m=1-12, preferably 2-6, with a straight or branched alkyl chain;

(CH₂)ₙCONHR⁸, with n=1-12, preferably 1-5; R⁸=H, C₁-C₆ alkyl, (CH₂)ₘN₃, or (CH₂)ₘ—N-maleimido, (CH₂)ₘ—NH—COCH₂X (X=Br or I), with m=1-12, preferably 2-6, and with straight or branched alkyl chains in (CH₂)n, (CH₂)ₘ and R⁶;

Groups R¹, R², R³, R⁴, R⁵, preferably R¹, R², R³ may be represented by a primary amino group forming aryl hydrazines Ar—NHNH₂ wherein Ar denotes the dye residue of Formula A that includes aryl amino groups and linkers;

a hydroxyl group, preferably R² or R³ being a hydroxy group forming aryl hydroxylamines Ar—NH₂OH wherein Ar denotes the dye residue of Formula A that includes aryl amino groups and linkers

further, one of the residues R¹, R², R³, R⁴, R⁵ may represent CH₂-C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂, or CH₂—C₅H₃N—NH₂, with C₅H₃N being pyridin-2,4-diyl, pyridin-2,5-diyl, pyridin-2,6-diyl, or pyridin-3,5-diyl;

additionally, R²-R³ and/or (R⁴-R⁵) may form a four-, five, six-, or seven-membered cycle, or a four-, five, six-, or seven-membered cycle with or without a primary amino group NH₂, secondary amino group NHR^{a}, where R^{a}=C₁-C₆ alkyl, a hydroxyl group OH, or a phosphorylated hydroxyl group —OP(O)(OH)₂ attached to one of the carbon atoms in this cycle;

optionally R²-R³ and/or (R⁴-R⁵) may form a four-, five, six-, or seven-membered heterocycle with an additional 1-3 heteroatoms, such as 0, N or S included into this heterocycle;

further, R¹ may represent an unsubstituted phenyl group, a phenyl group with one or several electron-donor substituents chosen from the set of OH, SH, NH₂, NHR^{a}, NR^{a}R^{b}, R^{a}O, R^{a}S, where R^{a} and R^{b} are independent from each other and may be C₁-C₆ alkyl groups with straight or branched carbon chains, a phenyl group with one or several electron-acceptors chosen from the set of N₀₂, CN, COH, COOH, CH═CHCN, CH═C(CN)₂, SO₂R^{a}, COR^{a}, COOR^{a}, CH═CHCOR^{a}, CH═CHCOOR^{a}, CONHR^{a}, SO₂NR^{a}R^{b}, CONR^{a}R^{b}, where R^{a} and R^{b} are independent from each other and may be H, or C₁-C₆ alkyl group(s) with straight or branched carbon chains; or R¹ may represent a heteroaromatic group.

Compounds of Formula A can exist and can be used as salts, solvates and hydrates, preferably as salts with alkaline metal cations including Na⁺, Li⁺, K⁺ and organic ammonium;

with the proviso that in all compounds of Formula A above at least two, preferably at least 3, 4, 5 or 6 negatively charged groups are present under basic conditions, i.e. 7<pH<14, and these negatively charged groups represent at least partially deprotonated residues of ionizable groups selected from the following: SH, COOH, a sulfonic acid residue SO₃H, a primary phosphate group OP(O)(OH)₂, a secondary phosphate group OP(O)(OH)R^{a}, where R^{a}=C₁-C₄ alkyl or substituted C₁-C₄ alkyl, a primary phosphonate group P(O)(OH)₂, a secondary phosphonate group P(O)(OH)R^{a}, where R^{a}=C₁-C₄ alkyl or substituted C₁-C₄ alkyl;

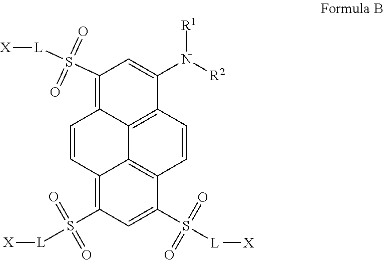

wherein R¹ and/or R² are independent from each other and may represent:

H, CH₃, C₂H₅, a linear or branched C₃-C₁₂ alkyl or perfluoroalkyl group, or a substituted C₂-C₆₁₂ alkyl group; in particular, (CH₂)ₙCOOR³, where n=1-12, preferably 1-5, R³ may be H, alkyl, in particular C₁-C₆, CH₂CN, benzyl, 2- and 4-nitrophenyl, fluorene-9-yl, polyhalogenoalkyl, polyhalogenophenyl, e.g. tetra- or penta-fluorophenyl, pentachlorophenyl, N-succinimidyl, sulfo-N-succinimidyl, 1-oxybenzotriazolyl or other potentially nucleophile-reactive leaving groups, and the alkyl chain in (CH₂)ₙ may be straight or branched; and

R¹-R² may form a four-, five, six-, or seven-membered non-aromatic carbocycle with an additional primary amino group NH₂, secondary amino group NHR^{a}, where R^{a}=C₁-C₆ alkyl, or hydroxyl group OH attached to one of the carbon atoms in this cycle; optionally R¹-R² may form a four-, five, six-, or seven-membered non-aromatic heterocycle with an additional heteroatom such as O, N or S included into this heterocycle;

a hydroxyalkyl group (CH₂)ₘOH, where m=1-12, preferably 2-6, with a straight or branched alkyl chain; one of R¹ or R² groups may be a carbonate or carbamate derivative (CH₂)ₘOOOOR⁴ or COOR⁴, where m=1-12 and R⁴=methyl, ethyl, 2-chloroethyl, N-succinimidyl, sulfo-N-succinimidyl, 1-oxybenzotriazolyl, a phenyl group or substituted phenyl group, e.g., 2- or 4-nitrophenyl, pentachlorophenyl, pentafluorophenyl, 2,3,5,6-tetrafluoro-phenyl, 2-pyridyl, or 4-pyridyl;

(CH₂)ₘNR^{a}R^{b}, where m=1-12, preferably 2-6, with a straight or branched alkyl chain; R^{a}, R^{b} are independent from each other and may be H, or optionally substituted C₁-C₄ alkyl group(s), in particular, one of R¹ or R² groups may be an alkyl azide group (CH₂)ₘN₃ with m=2-6 and a straight or branched alkyl chain; one of R¹ or R² may be (CH₂)ₙSO₂NR⁵NH₂ with n=1-12, while the substituent R⁵ can be represented by H, alkyl, hydroxyalkyl or perfluoroalkyl groups C₁-C₁₂;

one of R¹ or R² groups may be a primary amino group to form aryl hydrazines Ar—NR⁶NH₂ where Ar is the entire pyrene residue in Formula B and R⁶=H or alkyl; one of R¹ or R² groups may be a hydroxy group to form aryl hydroxylamines Ar—NR⁷OH where Ar is the entire pyrene residue in Formula B and R⁷=H or alkyl;

one of R¹ or R² groups may contain a terminal alkyloxyamino group (CH₂)ₙONH₂ with n=1-12, which can be linked via one or multiple alkylamino (CH₂)ₘNH or alkylamido (CH₂)ₘCONH groups in all possible combinations with m=0-12;

one of R¹ or R² groups may be CO(CH₂)ₙCOOR⁸, with n=1-5 and a straight or branched alkyl chain (CH₂)ₙ and with R⁸ selected from H, straight or branched C₁-C₆ alkyl, CH₂CN, 2- and 4-nitrophenyl, 2,3,5,6-tetrafluorophenyl, pentachlorophenyl, pentafluoro-phenyl, N-succinimidyl;

further, one of R¹ or R² may be (CH₂)ₙCONHR⁹, with n=1-5 and R⁹=H, C₁-C₆ alkyl, (CH₂)ₘN₃, (CH₂)ₘ—N-maleimido, (CH₂)ₘ—NHCOCH₂X (X=Br or I), where m=2-6 and with straight or branched alkyl chains in (CH₂)n and R⁹;

or one of R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl; or one of R¹ or R² may be an alkyl azide (CH)N₃ or alkine, in particular propargyl;

the linker L comprises at least one carbon atom and may comprise alkyl, heteroalkyl, in particular alkyloxy such as CH₂OCH₂, CH₂CH₂ OCH₂CH₂OCH₂, alkylamino or dialkylamino, particularly diethanolamine or N-methyl (alkyl) monoethanolamine moieties such as N(CH₃)CH₂CH₂O— and N(CH₂CH₂O—)₂, perfluoroalkyl, like single or multiple difluoromethyl (CF₂), alkene or alkyne moieties in any combinations, at any occurrence, linear or branched, with the length ranging from C₁ to C₁₂;

the linker L may also include a carbonyl (CH₂CO, CF₂CO) moiety;

X denotes a solubilizing and/or ionizable anion-providing moiety, in particular consisting of or including a moiety selected from the group comprising hydroxyalkyl (CH₂)ₙOH, thioalkyl ((CH₂)ₙSH), carboxy alkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12, or an analogon thereof wherein one or more of the CH₂ groups are replaced by CF₂,

further, the anion-providing moieties may be linked by means of non-aromatic O, N and S-containing heterocycles, e. g., piperazines, pipecolines, or, alternatively, one of the groups X may bear any of the moieties listed above for groups R¹ and R², also with any type of linkage listed for group L, and independently from other substituents;

Compounds of Formula B can exist and can be used as salts, solvates and hydrates, preferably as salts with alkaline metal cations including Na⁺, Li⁺, K⁺, NH₄⁺ and organic ammonium or organic phosphonium cations.

In more specific embodiments, a fluorescent dye salt according to the present invention may comprise negatively charged acid groups, in particular sulfonate and/or phosphate groups, and counterions selected from inorganic or organic cations, preferably alkaline metal cations, ammonium cations or cations of organic ammonium or phosphonium compounds (such as trialkylammonium cations), and/or may comprise a positively charged group or a charge-transfer complex formed at the nitrogen site N(R1)R2 in the dye of Formulae A-D as well as a counterion, in particular selected from anions of a strong mineral, organic or a Lewis acid.

With the proviso that in all compounds represented by Formula B three or six negatively charged groups are present in the residues X of Formula B under basic conditions, i.e. 7<pH<14, and these negatively charged groups represent at least partially deprotonated residues of ionizable groups selected from the following: SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl is provided.

In another aspect, a method for an automated carbohydrate mixture composition pattern profiling comprising the steps of

a) providing a first sample containing a first unknown carbohydrate mixture composition;
b) labelling of said carbohydrate mixture composition with a first fluorescent label;
c) providing a second sample containing a second carbohydrate mixture composition labelled with a second fluorescent label which may be added optionally to said first sample;
d) generating electropherograms/chromatograms of the carbohydrate mixture composition of said sample composition using electrokinetic/chromatographic separation techniques combined with fluorescence or laser induced fluorescence detection;
e) analyzing the identity and/or differences between the carbohydrate mixture composition pattern profiles of the first and the second sample, wherein the first fluorescent label of the first sample is different to the second fluorescent label of the second sample and wherein at least one of the first fluorescent label and the second fluorescent label is a fluorescent dye as defined above of general Formula A or B, like of general Formula C or D as defined below.

In a further aspect, a method for an automated carbohydrate mixture composition pattern profiling comprising the steps of

a) providing a sample containing a first carbohydrate mixture composition;
b) labelling of said carbohydrate mixture composition with a first fluorescent label;
c) providing a second sample labelled with a second fluorescent containing a second carbohydrate mixture composition to be compared with;
d) generating electropherograms/chromatograms of the carbohydrate mixture composition of the first and second sample composition using electrokinetic/chromatographic separation techniques combined with fluorescence or laser induced fluorescence detection;
e) comparing the standard migration/retention time indice(s) calculated from the obtained electropherogram/chromatogram of the first sample and the second sample;
f) analyzing the identify and/or differences between the carbohydrate mixture composition pattern profiles of the first and second sample, wherein standard migration/retention time indice(s) of the carbohydrates present in the sample are calculated based on internal standards of known composition labelled with a third fluorescent label and
wherein one of the first and the second fluorescent label is a fluorescent dye as defined above having a structure of general Formula A or B, like of general Formula C or D as defined below.

In an embodiment of the above methods for an automated carbohydrate mixture composition pattern profiling, the second carbohydrate mixture composition is a known carbohydrate mixture composition having a known pattern profile.

The present invention aims to provide methods allowing the determination and/or identification of carbohydrates whereby the labelled sample to be analyzed containing at least one carbohydrate is combined with a standard composition added to said unknown carbohydrate mixture. The sample containing both, the unknown carbohydrate (mixture) and the standard composition are labelled with a first fluorescent label and a second fluorescent label. At least one of said fluorescent label is a new fluorescent dye as described herein of general Formula A or B, like of general Formula C or D as defined below.

In an embodiment of the present invention, the single sample may contain at least two different probes to be analyzed, namely two differently labelled carbohydrates or carbohydrate mixture compositions beside the standard composition. That is, the new fluorescent dyes described herein allow to determine or to profile or to identify different carbohydrates in a single sample in a single run. In particular, when applying the method for calibration of a multi wavelength fluorescence detection system according to the present invention first, the use of at least three or more, like at least four different fluorescent dyes is possible (see Tables 2 and 3).

The new fluorescent dye feature multiple negatively charged residues and an aromatic amino or hydrazine group attached to the fluorophore which is excitable e.g. with an argon ion laser in their ionized (deprotonated) form.

That is, the dyes according to the present invention allow an increased throughput and sensitivity. Embodiments using the new dyes as described herein include: An embodiment wherein the sample to be analyzed contains two different probes to be analyzed, one labelled e.g. with APTS while the other probe is labelled with a new dye. In addition, a standard, e.g. a carbohydrate standard or a base pair standard is provided which is labelled with a new dye. A further embodiment includes a sample containing three different probes to be detected together with a standard labelled with a new dye according to the present invention. Three probes present in the sample include one APTS labelled probe, and two probes labelled with the dyes according to the present invention whereby said dyes are selected in a way that they do not interfere with each other in the emission profile. A further embodiment refers to a sample containing three probes, one labelled with APTS and the other probes are labelled with two different new dyes being different in the emission spectra as well as a standard being an alignment standard labelled with a new dye as well. A further embodiment includes a sample containing four probes to be determined, namely, one probe being APTS labelled while the other three probes are labelled with different new dyes in combination with a standard, like a base pair standard.

The dyes are selected to minimize any crosstalk between wavelengths. Suitable combinations are described below.

The use of the dyes as described herein for labelling of the carbohydrates present in the probes to be analyzed in the sample allow an increased sensitivity. The dyes described herein are advantageous with respect to a spectral calibration of the instrument as well as increase of compounds or probes to be analyzed present in one sample. Said sample can be analyzed with one capillary. Thus, it is possible to reduce the number of capillary as well as to increase sensitivity and alignment properties.

Further by shifting the excitation wavelength to a larger wavelength (red shift) the sensitivity of the sample labelled with said dye can be increased. Further, the dyes as described herein have better quantum yield compared to APTS, thus, increasing sensitivity further.

In addition, due to the increased sensitivity and the reduced crosstalk between wavelengths, the method is more robust, more reproducible, also in long-term, more precise, more independent from run-parameters, sample, sample-matrix, instrument, operator, lab and place as well as time-point. This is particularly true for the aging of the capillary and the gel. Differences from run to run over short-term or midterm as well as long-term can be compensated by the internal standard as described. Further, based on the method of calibration described herein and in combination with the new dyes, a more precise alignment is possible. Thus, it is possible to use the capillaries and columns for a longer time overcoming the problem of ageing which typically changes the migration/retention times of the samples. In addition, the capillary/column itself can be changed (e.g. shortened, thus, the analysis time can be shortened as well), without changing the aligned migration/retention times.

Moreover, it is possible to run the samples on the capillary with different instruments as well as under different run-parameter conditions like temperature, voltage, etc. This is demonstrated in the samples below. To summarize, the new dyes allow an increased throughput and sensitivity and enables also use of internal alignment for migration and retention times. The herein described electrokinetic and/or chromatographic separation-based glycoanalysis method allows the use of a universal (carbohydrate-based) alignment standard enabling aligned migration/retention times, independent from environmental factors like system, operator, matrix, etc.

In particular, the dyes as defined herein represent dyes which emit light with the maximum that is considerably shifted from that of APTS labelled analogs. Thus, detection of both fluorescent dyes or even of three of our different fluorescent dyes at the same time is possible without, respectively with minimal interference of said dyes between each other. The fluorescent dye as described herein is typically a multiple negative net charge dye which are especially high in the phosphorylated derivatives having negative charge of −4 and −6, providing higher electrophoretic mobility of the dye when conjugated with glycoconjugates compared to APTS glycoconjugates.

In the present invention, the term “carbohydrate(s)” refers to monosaccharide(s), like xylose arabinose, glucose, galactose, mannose, fructose, fucose, N-acetylglucoseamine, N-acetylgalactosamine, sialic acids; (homo or hetero) disaccharide(s), like lactose, sucrose, maltose, cellobiose; (homo or hetero) oligosaccharide(s), like glycans (e.g. N- and O-glycans), galactooligosaccharides (GOS), fructo-oligosaccharides (FOS), milk oligosaccharides (MOS) or even the glycomoiety of glycolipids; and (homo or hetero) polysaccharide(s), like amylose, amylopektin, cellulose, glycogen, glycosaminoglycans (GAG), or chitin. Oligo- and polysaccharides can either be linear or (multiple) branched.

The term “glycoconjugate(s)” as used herein means compound(s) containing a carbohydrate moiety, examples for glycoconjugates are glycoproteins, glycopeptides, proteoglycans, peptidoglycans, glycolipids, GPI-anchors, lipopolysaccharides.

The term “carbohydrate mixture composition pattern profiling” as used in means establishing a pattern specific for the examined carbohydrate mixture composition based on the number of different carbohydrates present in the mixture, the relative amount of said carbohydrates present in the mixture and the type of carbohydrate present in the mixture and profiling said pattern e.g. in a diagram or in a graphic, e.g. as an electropherogram, respectively, chromatogram. Thus, fingerprints illustrated e.g. in form of an aligned electropherogram/chromatogram, graphic, or diagram are obtained. For example, glycosylation pattern profiling based on fingerprints fall into the scope of said term. In this connection, the term “fingerprint” as used herein refers to aligned electropherograms and/or chromatograms being specific for a carbohydrate or carbohydrate mixture, a diagram or a graphic.

The term “quantitative determination” or “quantitative analysis” refers to the relative and/or absolute quantification of the carbohydrates. Relative quantification can be done straight forward via the individual peak heights of each compound, which corresponds linear (within the linear dynamic range of the FLR- and/or LIF-detector) to its concentration. The relative quantification outlines the ratio of each of one carbohydrate compound to another carbohydrate compound(s) present in the composition or the standard. Further, absolute (semi-)quantitative analysis is possible.

The internal carbohydrate standards of known composition, e.g. can be a set of mono, di- tri- tetra- and/or pentamers, linear and/or branched up to 40mers (or higher), eluting/migrating throughout the whole range of the fingerprints of the carbohydrate samples to be analyzed, but being detected in another wavelength trace/channel, as they are fluorescently labelled with another tag than the carbohydrate samples that is emitting at another wavelength and thus, don't show up in the samples trace/channel.

Examples are:
a. Carbohydrate based homo-polymers comprising pentoses (like xylose or arabinose), hexoses (like glucose, galactose or mannose) and hexosamines (like glucosamine, galactosamine, N-acetyl-glucosamine or N-acetyl-galactosamine) with a length of n=1 till 40 (or higher) and a glycosidic linkage in α1-2 (mannose oligosaccharides), α1-4 (e.g. maltose, starch), α1-5 (arabino-oligosaccharides), α1-6 (e.g. dextran, pullulan, starch), α1-3 (e.g. dextran, pullulan), β1-3 (e.g. cellobiosyl-glucose), β1-4 (e.g. cellulose, mannan, xylo-oligosaccharides, chitosan), and β1-6
b. hetero oligo-polymers like hemicelluloses, arabinoxylan, arabinogalactan, fructane
c. N-glycans
d. O-glycans
e. Glycolipids
f. Milk oligosaccharides (MOS)

The present invention represents a further development of the method described in EP 2112506 A1, US 2009/0288951 A1 and counterparts thereof. In particular, with the new dyes as identified herein, it is possible to use a (internal) standard identical or similar to the sample, as both are now carbohydrate(s), respectively carbohydrate mixture(s) with the same, respectively, similar properties (e.g. size, mass, charge, hydrophilicity, hydrophobicity, etc.) and thus show the same, respectively, similar behavior with changing environment, like different matrices (e.g. content and composition of salts, solvents, gel, etc.) but also temperature and time (which are also causing changes of the matrix, e.g. due to gel-ageing). Thus, highly reproducible and precisely aligned migration/retention times allow a highly reliable identification of carbohydrates via migration/retention time matching via a respective database, containing carbohydrates and their respective aligned migration/retention times.

This allows to identify unknown carbohydrates and unknown glycosylation pattern profiles with higher sensitivity and specificity. This is particularly true for complex carbohydrate preparations and glycosylation pattern.

The term “substituted” as used herein, generally refers to the presence of one or more substituents, in particular substituents selected from the group comprising straight or branched alkyl, in particular C₁-C₄ alkyl, e.g. methyl, ethyl, propyl, butyl; isoalkyl, e.g. isopropyl, isobutyl (2-methylpropyl); secondary alkyl group, e.g. secbutyl (but-2-yl); tert-alkyl group, e.g. tert-butyl (2-methylpropyl). Additionally, the term “substituted” may refer here to alkyl groups having at least one deuterium-, fluoro-, chloro- or bromo substituents instead of hydrogen atoms, or methoxy, ethoxy, 2-(alkyloxy)ethyloxy groups (AlkOCH₂CH₂O), and, in a more general case, oligo(ethylenglycol) residues of the art Alk(OCH₂CH₂)ₙOCH₂CH₂—, where Alk=CH₃, C₂H₅, C₃H₇, C₄H₁₀, and n=1-23.

The terms “aromatic heterocyclic group” or “heteroaromatic group”, as used herein, generally refer to an unsubstituted or substituted cyclic aromatic radical (residue) having from 5 to 10 ring atoms of which at least one ring atom is selected from S, O and N; the radical being joined to the rest of the molecule via any of the ring atoms. Representative, but not limiting examples are furyl, thienyl, pyridinyl, pyrazinyl, pyrimidinyl, pyrrolyl, imidazolyl, thiazolyl, oxazolyl, isooxazolyl, thiadiazolyl, oxadiazolyl, quinolinyl and isoquinolinyl.

Compounds of the general structural Formula A above are acridone dyes, compounds of the Formula B above are pyrene dyes.

More specifically, according to the IUPAC rules the compounds of Formula A are 7-aminoacridon-2-sulfonamides, whereas the compounds of Formula B are 1-aminopyrene dyes with functionally substituted sulfonyl groups in positions 3, 6, 8, i.e. (functionally substituted) 1,6,8-trisulfonyl-3-aminopyrenes, as shown in the basic structural Formulae A and B in Scheme below.

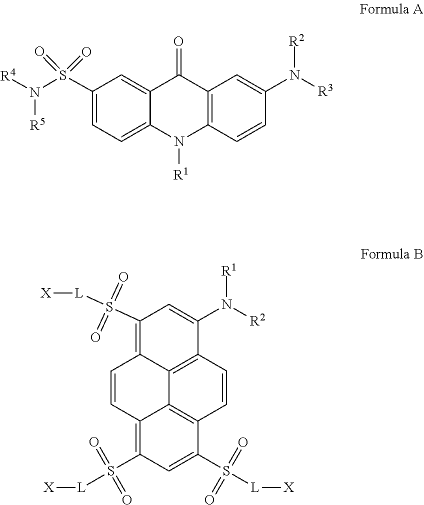

The novel fluorescent dyes of the present invention exhibit a number of favorable characteristics:
aromatic amino (NH₂), hydrazine (NRNH₂), hydrazide (CONRNH₂), hydroxylamine (NROH), reactive carbamate (NHCOOR) or alkoxyamino group (RONH₂) for efficient and clean reductive amination at e.g. pH ˜ 2-5 or direct condensation with carbohydrates; preferably, the aromatic amino group is primary, but it can also be a secondary one; see Scheme above for structures
large net charges in conjugates—in the range of −3 to −12 at pH at least from 7 to 14
very good solubility in aqueous media at a wide range of pH;
high brightness (which is the overall result of the fluorescence quantum yield and extinction)
exceptional stability of the dye core, e.g. against reduction with borane-based reagents
the ability to be exited with an argon ion laser emitting at 488 and 514 nm with a perfect spectral match and high fluorescence quantum yields.
minimal emission at ca. 520 nm
The dyes are amenable to purification up to 99%.

The novel fluorescent tags of the invention even allow the detection of “heavy” glycans with very long migration times. Due to these long migration times and peak-broadening, such “heavy” glycans are very difficult to detect electrokinetically; especially if APTS is used as fluorescent tag.

In the following, more specific embodiments of the present invention are described.

In Formula A above, NR¹ and/or N(R²)R³ preferably comprise carbonyl- or nucleophile-reactive groups. R¹, R², and R³ can be represented by H, linear or branched alkyl, hydroxyalkyl or perfluoroalkyl groups. Substituents R³, R⁴ and R⁵ preferably comprise solubilizing and/or anion-providing groups, particularly hydroxyalkyl ((CH₂)ₙOH), thioalkyl ((CH₂)ₙSH), carboxyalkyl ((CH₂)ₙ CO₂H), alkyl sulfonate ((CH₂)ₙSO₃H), alkyl sulfate ((CH₂)ₙOSO₃H), alkyl phosphate ((CH₂)ₙOP(O)(OH)₂) or alkyl phosphonate ((CH₂)ₙP(O)(OH)₂), wherein n is an integer ranging from 1 to 12.

Alternatively, substituents R¹, R², R³, R⁴ and R⁵ may be represented by carboxylic acid residues (CH₂)ₙCOOH, where n=1-12, and their reactive esters (CH₂)ₙCOOR⁶ as nucleophile-reactive groups. R⁶ can be H, alkyl, (tert-butyl including), benzyl, fluorene-9-yl, polyhalogenoalkyl, CH₂CN, polyhalogenophenyl (e. g., tetra- or pentafluorophenyl, pentachlorophenyl), 2- and 4-nitrophenyl, N-succinimidyl, sulfo-N-succinimidyl, 1-oxybenzotriazolyl or other potentially nucleophile-reactive leaving groups. The alkyl chains (or backbones) (CH₂)ₙ may be linear or branched.

Further, the aryl amino groups (NR¹ and NR²R³) in Formula A can be connected to an analyte-reactive group via (poly)methylene, carbonyl, nitrogen or sulfur-containing linear or branched linkers, particularly (CH₂)ₘCON(R⁷), CO(CH₂)ₘN(R⁷), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO, their combinations, or linked as a part of nitrogen-containing non-aromatic heterocycles (e.g., piperazines, pipecolines, oxazolines); m and n are integers ranging from 0 to 12 or 1 to 12. The substituent R⁷ may be represented by any of the functional groups listed for R¹, R², R³, R⁴ and R⁵ above.

Substituents R¹, R² and R³ in Formula A may be also represented by a primary amino group, thus comprising carbonyl-reactive aryl hydrazines, (R²=H, R¹ or R³=NH₂ or R¹=NH₂, R², R³=alkyl, perfluoroalkyl or alkyl) conjugated or substituted with solubilizing and/or anion-providing moieties, listed as possible candidates for R⁴ and R⁵, particularly: hydroxyalkyl (CH₂)ₙOH, thioalkyl ((CH₂)ₙSH), carboxyalkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12 or 1 to 12. Alternatively, hydrazine derivatives might be represented by sulfonyl hydrazides, where R⁴=NH₂, while R⁵ are alkyl, perfluoroalkyl or alkyl groups decorated with solubilizing and/or anion-providing groups of the types mentioned above.

Alternatively, aryl amino groups (NR¹ and/or NR²R³) in Formula A can be connected to an acyl hydrazine or alkyl hydrazine moiety indirectly, via linkers, thus comprising hydrazides (ZCONHNH₂) or hydrazines (ZNHNH₂), respectively. Here Z denotes the dye residue of Formula A that includes aryl amino groups and linkers. In particular, R¹ and R² may be represented by: (CH₂)ₘCON(R⁷), CO(CH₂)ₘN(R⁷), CO(CH₂)ₘS(CH₂)ₙ, (CH₂)ₘS(CH₂)ₙCO, CO(CH₂)ₘSO₂(CH₂)ₙ, (CH₂)ₘSO₂(CH₂)ₙCO and their combinations; m and n are integers ranging from 0 to 12. Substituent R⁷ can be represented by any of the functional groups for R¹, R² R³, R⁴ and R⁵ that are listed above as candidates for functional groups R¹—R⁵, particularly: hydroxyalkyl (CH₂)ₙOH, thioalkyl ((CH₂)ₙSH), carboxyalkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12 or 1 to 12. Linkers may also be represented by non-aromatic O, N and S-containing heterocycles (e. g., piperazines, pipecolines).

Further, R¹, R² and R³ may be represented by CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridine-2,4-diyl, pyridine-2,5-diyl, pyridine-2,6-diyl, pyridine-3,5-diyl.

The analyte-reactive group at variable positions R¹, R² R³, R⁴ and R⁵ may be represented by an aromatic or heterocyclic amine, carboxylic acid, ester of the carboxylic acid (e.g., N-hydroxysuccinimidyl or another amino reactive ester); or represented by alkyl azide (CH₂)ₙN₃, alkine (propargyl), amino-oxyalkyl (CH₂)ₙONH₂, maleimido (C₄H₃NO₂ with a nucleophile-reactive double bond) or halogeno ketone function (COCH₂X; X=Cl, Br and I), as well as halogeno amide group (NRCOCH₂X, R=H, C₁-C₆-alkyl, X=Cl, Br, I) connected either directly or indirectly via carbonyl, amido, nitrogen, oxygen or sulfur-containing linkers listed for hydrazine derivatives where n=1-12.

According to some more preferred embodiments of the present invention, the substituent R¹ in the above Formula A is defined as follows:

R¹ in Formula A represents hydrogen, a lower alkyl group (C₁-C₄), an unsubstituted phenyl group, a phenyl group with one or several electron-donor substituents chosen from the set of OH, SH, NH₂, NHR^{a}, NR^{a}R^{b}, R^{a}O, R^{a}S, OP(O)(OR^{a})(OR^{b}) where R^{a} and R^{b} are independent from each other and may be C₁-C₁₂, preferably C₁-C₆, alkyl groups with linear or branched chains, a phenyl group with one or several electron-acceptors chosen from the set of NO₂, CN, COH, COOH, CH═CHCN, CH═C(CN)₂, SO₂R^{a}, SO₃R^{a}, COR^{a}, COOR^{a}, CH═CHCOR^{a}, CH═CHCOOR^{a}, CONHR^{a}, SO₂NR^{a}R^{b}, CONR^{a}R^{b}, P(O)(OR^{a})(OR^{b}) where R^{a} and R^{b} are independent from each other and may be H, or C₁-C₆ alkyl group(s) with straight or branched carbon chains; alternatively, R¹ may represent an aromatic heterocyclic group, in particular, 2-pyridyl, 3-pyridyl, 4-pyridyl, 2-thienyl, 3-thienyl, pyrimidin-4-yl, pyrimidin-2-yl, pyrimidin-5-yl, or other electron acceptor groups derived from aromatic heterocycles, such as 4-pyridyl-N-oxides, N-alkylpyridinium salts, or betaines, in particular, N-(o-sulfoalkyl)-4-pyridinium, N-(o-sulfoalkyl)-2-pyridinium, N-(1-hydroxy-4,4,5,5-tetrafluoro-cyclopent-1-en-3-on-2-yl)-4-pyridinium, N-(1-hydroxy-4,4,5,5-tetrafluorocyclopent-1-en-3-on-2-yl)-2-pyridinium.

In particular, R¹ may represent a positively charged heterocyclic group derived from 2-pyridyl, 3-pyridyl, or 4-pyridyl precursors with an 7-aminoacridon-2-sulfonamide backbone and alkylating agents (e.g. alkyl halides, alkyl sulfonates, alkyl triflates, 1,3-propanesulton, 1,4-butanesulton) or electrophiles (e. g., perfluorocyclopentene).

Especially preferred are aminoacridone-containing compounds of the structural Formula A above that have one of the following formulae:

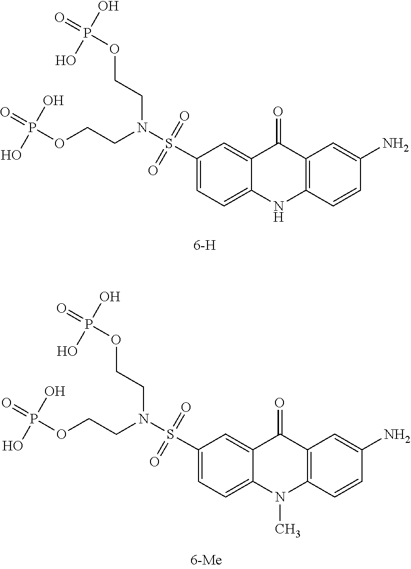

In Formula B, L is a divalent linker that connects the dye core with solubilizing and/or ionizable moieties and also tailors the spectral properties.

Typically, it presence results in considerable bathofloric and bathochromic shifts accompanied by a better match to the 488 nm commercial lasers, as compared to APTS dye tag, where fragment L is absent and group X is OH.

The linker L comprises or consists of at least one carbon atom and can represent alkyl, heteroalkyl (e. g., alkyloxy: CH₂OCH₂, CH₂CH₂ OCH₂CH₂OCH₂), difluoromethyl (CF₂), alkene or alkine moieties in any combinations, at any occurrence, linear or branched, with the length ranging from C₁ to C₁₂. The linker can also include a carbonyl (CH₂CO, CF₂CO) and Sulfonamides are the case when L is an alkylamino or a dialkylamino group, particularly diethanolamine or N-methyl (alkyl) monoethanolamine moieties (i.e., N(CH₃)CH₂CH₂O— and N(CH₂CH₂O—)₂), which allow further connection to a solubilizing and/or ionizable moieties X. Certain embodiments of this invention represent the combination of moieties L and X according to the formulae (CH₂)₃OP(O)(OH)₂ and N(CH₃)(CH₂)₂OP(O)(OH)₂. The sulfonamides of this type thus have general formula SO₂NR³R⁴, where R³ and R⁴ are independent from each other and can be represented by H, alkyl, heteroalkyl (e. g., alkyloxy: CH₂OCH₂, CH₂CH₂O, CH₂CH₂OCH₂), difluoromethyl (CF₂) in any combinations, linear or branched, with the length ranging from C₁ to C₁₂, also bearing terminal OH groups.

N(R¹)R² in Formula B preferably comprises a carbonyl- or nucleophile-reactive group. Substituents R¹ and R² are independent from each other and can be both represented by hydrogen. One of those can be a linear or branched alkyl (perfluoroalkyl) group C₁-C₁₂. At the same time, one of R¹ and R² may be represented by carboxylic acid residues (CH₂)ₙCOOH and their regular or reactive esters (CH₂)ₙCOR⁵ where n is an integer ranging from 1 to 12. The residue R⁵ is H, alkyl, (tert-butyl including), benzyl, fluorene-9-yl, polyhalogenoalkyl, CH₂CN, polyhalogenophenyl (e. g., tetra- or pentafluoro phenyl, pentachlorophenyl), 2- and 4-nitrophenyl, N-sucinimidyl, sulfo-N-sucinimidyl or other potentially nucleophile-reactive leaving groups. The alkyl chains (or backbones) (CH₂)ₙ may be linear or branched. Particularly, the formula can be depicted as Z—NR¹(CH₂)ₙCOR⁵, where Z is the rest of the molecule in Formula B that also includes groups L and X.

Further, the nucleophile-reactive group COR⁵ can be connected to the aryl amino group N(R¹)R² via (poly)methylene, oxymethylene (CH₂OCH₂, CH₂CH₂OCH₂, PEG) carbonyl, carbonate, urethane, nitrogen or sulfur-containing linkers (spacers) branched or linear, particularly (CH₂)ₘCON(R⁶), CONH(CH₂)ₙ, (CH₂)ₘOCONH(CH₂)ₙ, CO(CH₂)ₙ, CO(O)NR⁶, (CH₂)ₘSO₂ₘN(R⁶), CO(CH₂)ₘS(CH₂)ₙ, (CH₂)ₘS(CH₂)ₙCO, CO(CH₂)ₘSO₂(CH₂)ₙ, (CH₂)ₘSO₂NR⁶, and their combinations; m and n are integers ranking from 0 to 12. The reactive group R⁵ can be linked by means of non-aromatic O, N and S-containing heterocycles (e. g., piperazines, pipecolines, oxazolines). Substituent R⁶ might be represented by H, alkyl, hydroxyalkyl or perfluoroalkyl groups C₁-C₁₂.

One of the the substituents R¹ and R² in Formula B may be represented by a primary amino group, thus comprising carbonyl-reactive aryl hydrazines (R¹=NH₂, R²=alkyl, perfluoroalkyl) or by a hydroxyl group to form aryl oximes (ArNHOH). Alternatively, the alkyl hydrazine or oxime reactive moiety in Formula B can be connected to aryl amino group N(R¹)R² via linkers listed above for the reactive group R⁴. Sulfonyl hydrazides constitute a special case when R¹ or R²=(CH₂)ₙSO₂NR⁶NH₂ with n=1-12, while the substituent R⁶ can be represented by H, alkyl, hydroxyalkyl or perfluoroalkyl groups C₁-C₁₂. The sulfonylamide (sulfonamide, sulfamide) group can be also attached via diverse linkers listed above for the case with the reactive groups R³, R⁴ and R⁵.

Further, R¹ and R² may be represented by CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridine-2,4-diyl, pyridine-2,5-diyl, pyridine-2,6-diyl, pyridine-3,5-diyl.

Substituents R¹ and R² may be also represented by alkyl azide (CH₂)ₙN₃, alkine (propargyl), maleimido (C₄H₃NO₂ with a nucleophile-reactive double bond) or halogeno-ketone function (COCH₂X; X=C₁, Br and 1) connected either directly or via carbonyl, amido, nitrogen or sulfur-containing linkers listed for hydrazine derivatives; n=1-12.

Group X in Formula B denotes solubilizing and/or ionizable anion-providing moieties, particularly the ones that provide enhanced electrophoretic mobility. Group X can include hydroxyalkyl (CH₂)ₙOH, thioalkyl ((CH₂)ₙSH), carboxy alkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12. Alternatively, the CH₂ group can be replaced by CF₂. The anion-providing moieties can be also linked by means of non-aromatic O, N and S-containing heterocycles (e.g., piperazines, pipecolines). Alternatively, one of the groups X can bear any of the carbonyl- or nucleophile-reactive moieties listed for groups R¹ and R², also with any type of linkage listed for group L, and independently from other substituents. Compounds of Formula B can exist and be applied in the form of salts that involve all possible types of cations, preferably Na⁺, K⁺, Li⁺ or trialkylammonium.

The fluorescent dyes of Formula B may be present in form of salts, solvates or hydrates, in particular, salts with cations including Na⁺, K⁺, Li⁺, NH₄⁺ and organic ammonium or organic phosphonium cations.

According to one specific embodiment of the invention, the anion-providing group(s) X may represent, at each occurrence in Formula B, one to four groups SO₃H attached to the linker group L, as indicated by the term (SO₃H)ₙ with n=1-4 in Formula B of claim 3.

According to a specific embodiment of the invention, the compounds of the structural Formula B above are alkylsulfonyl derivatives of Formula C

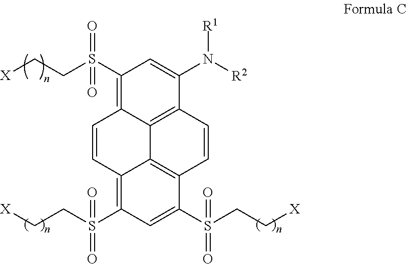

wherein

R¹ and/or R² are independent from each other and may represent:

H, CH₃, C₂H₅, a straight or branched C₃-C₁₂, preferably C₃-C₆, alkyl group, or a substituted C₂-C₁₂, preferably C₂-C₆, alkyl group; in particular, (CH₂)ₙCOOR³, where n=1-12, preferably 1-5, R³ may be H, CH₂CN, 2- and 4-nitrophenyl, 2,3,5,6-tetrafluorophenyl, pentachlorophenyl, pentafluorophenyl, N-succinimidyl, sulfo-N-succinimidyl, 1-oxybenzotriazolyl and the alkyl chain in (CH₂)ₙ may be straight or branched; and

R¹-R² may form a four-, five, six-, or seven-membered non-aromatic carbocycle with an additional primary amino group NH₂, secondary amino group NHR^{a}, where R^{a}=C₁-C₆ alkyl, or hydroxyl group OH attached to one of the carbon atoms in this cycle; optionally R¹-R² may form a four-, five, six-, or seven-membered non-aromatic heterocycle with an additional heteroatom such as O, N or S included into this heterocycle; a hydroxyalkyl group (CH₂)ₘOH, where m=1-12, preferably 2-6, with a straight or branched alkyl chain; one of R¹ or R² groups may be a carbonate or carbamate derivatives where one of R¹ or R² groups is (CH₂)ₘOCOOR⁴ or COOR⁴, where m=1-12 and R⁴=methyl, ethyl, 2-chloroethyl, N-succinimidyl, sulfo-N-succinimidyl, 1-oxybenzotriazolyl a phenyl group or substituted phenyl group, e.g., 2- and 4-nitrophenyl, pentachlorophenyl, pentafluorophenyl, 2,3,5,6-tetrafluoro-phenyl, 2-pyridyl, or 4-pyridyl; (CH₂)ₘNR^{a}R^{b}, where m=1-12, preferably 2-6, with a straight or branched alkyl chain; R^{a}, R^{b} are independent from each other and may be H, or optionally substituted C₁-C₄ alkyl group(s), in particular, one of R¹ or R² groups may be an alkyl azide group (CH₂)ₘN₃ with m=2-6 and a straight or branched alkyl chain;

one of R¹ or R² groups may be (CH₂)ₙCOOR⁵, with n=1-5 and a straight or branched alkyl chain (CH₂)ₙ and with R⁵ selected from H, straight or branched C₁-C₆ alkyl, CH₂CN, 2- and 4-nitrophenyl, 2,3,5,6-tetrafluorophenyl, pentachlorophenyl, pentafluoro-phenyl, sulfo-N-succinimidyl, N-succinimidyl, 1-oxybenzotriazolyl; further, one of R¹ or R² may be (CH₂)ₙCONHR⁶, with n=1-12, preferably 1-5, and R⁶=H, C₁-C₆ alkyl, (CH₂)ₘN₃, (CH₂)ₘ—N-maleimido, (CH₂)ₘ—NHCOCH₂X (X=Br or I), where m=2-6 and with straight or branched alkyl chains in (CH₂)ₙ and R⁶; or one of R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂, or CH₂—C₅H₃N—NH₂, with C₅H₃N being pyridin-2,4-diyl, pyridin-2,5-diyl, pyridin-2,6-diyl, or pyridin-3,5-diyl; the (CH₂)ₙ—CH₂ linker, with n=1-5, between the S02 fragment and the residue X in Formula B may represent a straight-chain, branched or cyclic group having 2-6 carbon atoms;

X=SH, COOH, SO₃H, OP(O)(OH)₂, OP(O)(OH)R^{a}, where R^{a}=optionally substituted C₁-C₄ alkyl, P(O)(OH)₂, P(O)(OH)R^{a}, where R^{a}=optionally substituted C₁-C₄ alkyl;

with the proviso that in all compounds represented by Formula C three or six negatively charged groups are present in the residues X of Formula B under basic conditions, i.e. 7<pH<14, and these negatively charged groups represent at least partially deprotonated residues of ionizable groups selected from the following: SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl.

According to a more specific embodiment, of the invention, the fluorescent dye of the invention is represented by Formula C wherein X at each occurrence is SO₃H and n is 1-12, preferably 1-6, or a salt thereof.

According to another specific embodiment of the invention, the compounds of the structural Formula B above are sulfamide derivatives of Formula D

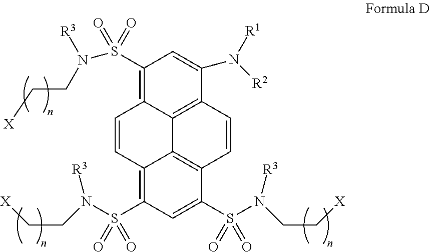

wherein

R¹ and/or R² are independent from each other and may represent H, CH₃, C₂H₅, or a straight or branched, optionally substituted, C₃-C₁₂, preferably C₃-C₆, alkyl group; in particular, (CH₂)ₙCOOR⁴, where n=1-12, preferably 1-5, R⁴ may be H, CH₂CN, 2- and 4-nitrophenyl, 2,3,5,6-tetrafluorophenyl, pentachlorophenyl, pentafluorophenyl, N-succinimidyl, sulfo-N-succinimidyl, 1-oxybenzotriazolyl, and the alkyl chain in (CH₂)ₙ may be straight or branched; and

R¹-R² may form a four-, five, six-, or seven-membered non-aromatic carbocycle with an additional primary amino group NH₂, secondary amino group NHR^{a}, where R^{a}=optionally substituted C₁-C₆ alkyl, or hydroxyl group OH attached to one of the carbon atoms in this cycle; or optionally R¹-R² may form a four-, five, six-, or seven-membered non-aromatic heterocycle with a heteroatom such as 0, N or S included into this heterocycle;

R¹ and/or R² may further represent:

a hydroxyalkyl group (CH₂)ₘOH, where m=1-12, preferably 2-6, with a straight or branched, optionally substituted alkyl chain; one of R¹ or R² groups may be a carbonate or carbamate derivative (CH₂)ₘOCOOR⁵ or COOR⁵, where m=1-12 and R⁵=methyl, ethyl, 2-chloroethyl, CH₂CN, N-succinimidyl, sulfo-N-succinimidyl, 1-oxybenzotriazolyl, a phenyl group or substituted phenyl group, such as 2- and 4-nitrophenyl, pentachlorophenyl, pentafluoro-phenyl, 2,3,5,6-tetrafluorophenyl, 2-pyridyl, 4-pyridyl; (CH₂)ₘNR^{a}R^{b}, where m=1-12, preferably 2-6, with a straight or branched alkyl chain; R^{a}, R^{b} are independent from each other and represent hydrogen and/or optionally substituted C₁-C₄ alkyl groups;
(CH₂)ₘN₃, m=1-12, preferably 2-6, with a straight or branched alkyl chain; (CH₂)ₙCONHR⁶, where n=1-12, preferably 1-5 and R⁶=H, substituted or unsubstituted C₁-C₆ alkyl, (CH₂)ₘN₃, (CH₂)ₘ—N-maleimido, (CH₂)ₘ—NHCOCH₂Y (Y=Br, I) where m=1-12, preferably 2-6, with straight or branched alkyl chains in (CH₂)ₙ and R⁶;
one of R¹ or R² groups may be a primary amino group to form aryl hydrazines Ar—NR⁷NH₂ where Ar is the entire pyrene residue in Formula D and R⁷=H or alkyl; one of R¹ or R² groups may be a hydroxy group to form aryl hydroxylamines Ar—NR⁸OH where Ar is the entire pyrene residue in Formula D and R⁸=H or alkyl;
one of R¹ or R² groups may contain a terminal alkyloxyamino group (CH₂)ₙONH₂ with n=1-12, which can be linked via one or multiple alkylamino (CH₂)ₘNH, alkylamido (CH₂)ₘCONH, alkyl ether or alkyl ester group(s) in all possible combinations with m=0-12;
further, R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl;
R³=H, (CH₂)_{q}CH₂X, C₂H₅, a straight or branched C₃-C₆ alkyl group, CₘH₂ₘOR, where m=2-6, with a straight or branched alkan-diyl chain CₘH₂ₘ, and R=H, CH₃, C₂H₅, C₃H₇, CH₃(CH₂CH₂O)ₖCH₂CH₂; with k=1-12; while the (CH₂)_{q}CH₂linker may represent a straight-chain, branched or cyclic group having 2-6 carbon atoms;
in Formula D, the (CH₂)ₙ—CH₂ linker, with n=1-12, preferably 1-5, between the sulfonamide fragment SO₂N and the residue X may represent a straight-chain, branched or cyclic group having 2-6 carbon atoms;
X=SH, COOH, SO₃H, OP(O)(OH)₂, OP(O)(OH)R^{a}, where R^{a}=substituted or unsubstituted C₁-C₄ alkyl, P(O)(OH)₂, P(O)(OH)R^{a}, where R^{a}=substituted or unsubstituted C₁-C₄ alkyl;
with the proviso that in all compounds represented by Formula D three, six, nine or twelve negatively charged groups are present in the residues X of Formula C under basic conditions, i.e. 7<pH<14, and these negatively charged groups represent at least partially deprotonated residues of ionizable groups selected from the following: SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl.

According to preferred embodiments of the invention, the substituents R¹ and R² in the above Formulae B, C and D are defined as follows:

R¹ and/or R² in Formula B represent H, CH₃, (CH₂)ₙCOOR³, where n=1-4, R³ may be H, CH₂CN, 2- or 4-nitrophenyl, 2,3,5,6-tetrafluorophenyl, pentachlorophenyl, pentafluorophenyl, N-succinimidyl, sulfo-N-succinimidyl, 1-oxybenzotriazolyl, while the alkyl chain in (CH₂)ₙ is straight; n=1-12.
Compounds of Formulae C and D can exist and be applied in the form of salts that involve all possible types of cations, preferably Na⁺, K⁺ or trialkylammonium cations.

Especially preferred aminopyrene-containing compounds of the general structural Formulae B, C and D above have one of the following formulae:

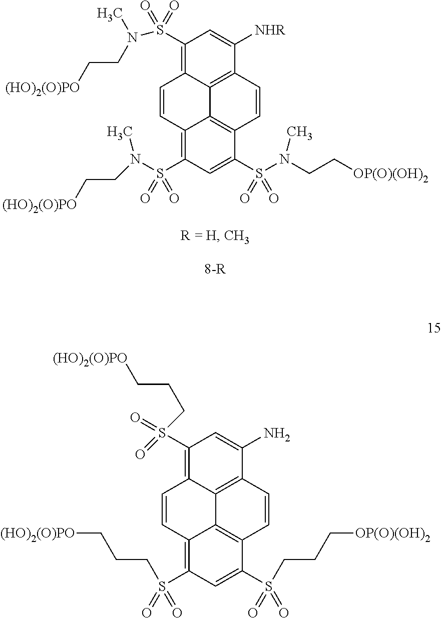

One preferred embodiment of the present invention relates to compounds Formulae A-B or A-D above, where the negative charges are provided by several primary phosphate groups, in particular, doubly O-phosphorylated 7-aminoacridon-2-sulfonamides (two phosphate groups), triple O-phosphorylated 1,6,8-tris[(ω-hydroxyalkyl)sulfonyl]-pyrene-3-amines (three phosphate groups), and 1,6,8-tris[N-(ω-hydroxyalkyl)sulfonylamido] pyrene-3-amines. These compounds possess superior brightness and a lot better electrophoretic mobilities, compared to APTS, and were successfully applied in labeling of glycans and analysis of the conjugates by capillary gel electrophoresis (CGE) with detection by laser induced fluorescence (LIF).

Another preferred embodiment of the present invention relates to compounds of Formula B, C or D where R¹ and/or R² represent: H, deuterium, alkyl or deutero-substituted alkyl, in particular alkyl or deutero-substituted alkyl with 1-12 C atoms, preferably 1-6 C atoms, wherein one, several or all H atoms of the alkyl group may be replaced by deuterium atoms, 4,6-dihalo-1,3,5-triazinyl (C₃N₃X₂) where halogen X is preferably chlorine, 2-, 3- or 4-aminobenzoyl (COC₆H₄NH₂), N-[(2-, N-[(3- or N-[(4-aminophenyl)ureido group (NHCONHC₆H₄NH₂), N-[(2-, N-[(3- or N-[(4-aminophenyl)thioureido group(NHCSNHC₆H₄NH₂ or linked carboxylic acid residues and their reactive esters of the general formulae (CH₂)ₘ₁COOR³, (CH₂)ₘ₁OCOOR³ (CH₂)ₙ₁COOR³ or (CO)ₘ₁(CH₂)ₘ₂(CO)ₙ₁(NH)ₙ₂(CO)ₙ₃(CH₂)ₙ₄COOR³ where the integers m1, m2 and n1, n2, n3, n4 independently range from 1 to 12 and from 0 to 12, respectively, with the chain (CH₂)_{m/n} being straight, branched, saturated, unsaturated, partially or completely deuterated, and/or or included into a carbo- or heterocylcle containing N, O or S, whereas R³ is H, D or a nucleophile-reactive leaving group, preferably including but not limited to N-succinimidyl, sulfo-N-succinimidyl, 1-oxybenzotriazolyl, cyanomethyl, polyhalogenoalkyl, polyhalogenophenyl, e.g. tetra- or pentafluorophenyl, 2- or 4-nitrophenyl.

The novel compounds of the invention have small molecular size and, in preferred embodiments, a drastically increased high negative net charge (z) is provided (such as, at least, z=−4 for phosphorylated acridones and at least z=−6 for phosphorylated pyrene dyes). These two requirements are equivalent to a low hydrodynamic radius and a low mass to charge ratio (m/z), respectively. As a result, high velocities and fast separations at good analytical resolution can be achieved in electrokinetic measurements for these compounds and the corresponding labeled carbohydrates.

The negative charges are provided by acidic groups which can be deprotonated in basic or even neutral media. Phosphate groups are preferred for this purpose, because primary alkyl phosphates (R—OPO₃H₂) have pKₐ values for the first and the second acidic protons in the range of 1-2 and 6-7, respectively. As a consequence, one single phosphate group can introduce two negative charges in buffer solutions under basic conditions (e.g., at pH above 8, R—OPO₃²⁻ is present). To achieve the negative charge of −4, the attachment of two phosphate groups is necessary, etc. However other acidic groups, in particular selected from the groups X as defined in Formulae A-B above are also suitable.

Generally, the compounds of Formulae A-B above are suitable and advantageous for the use as a fluorescent label for amino acids, peptides, proteins, including primary and secondary antibodies, single-domain antibodies, docetaxel, avidin, streptavidin and their modifications, aptamers, nucleotides, nucleic acids, toxins, lipids, carbohydrates, including 2-deoxy-2-aminoglucose and other 2-deoxy-2-aminoaminopyranosides, glycans, glucans, biotin, and other small molecules, e.g., jasplakinolide and its modifications.

Compounds 7-R (R=H, Me), 13a, 13b, 16 and 18 (see Scheme 7 below) possess free hydroxyl groups and are suitable as precursors for obtaining phosphorylated pyrene dyes of the general Formula B. In particular, compounds 7-R (R=H, Me) were phosphorylated and afforded dyes 8-R (R=H, Me). Compounds 13a,b and 18 were phosphorylated analogously. Thus, e.g. both precursor dyes 13a and 13b gave (after the basic work-up of the reaction mixture) compound 15. Compound 16 has a free carboxyl group which can be used a reactive center for bioconjugation. Thus, compound 16 represents a fluorescent label for amino acids, peptides, proteins, including primary and secondary antibodies, single-domain antibodies, docetaxel, avidin, streptavidin and their modifications, aptamers, modified nucleotides, modified nucleic acids containing an amino group, toxins, lipids, carbohydrates, including 2-deoxy-2-aminoglucose and other 2-deoxy-2-aminoaminopyranosides, modified biotin (e.g., biocytin), and other small molecules.

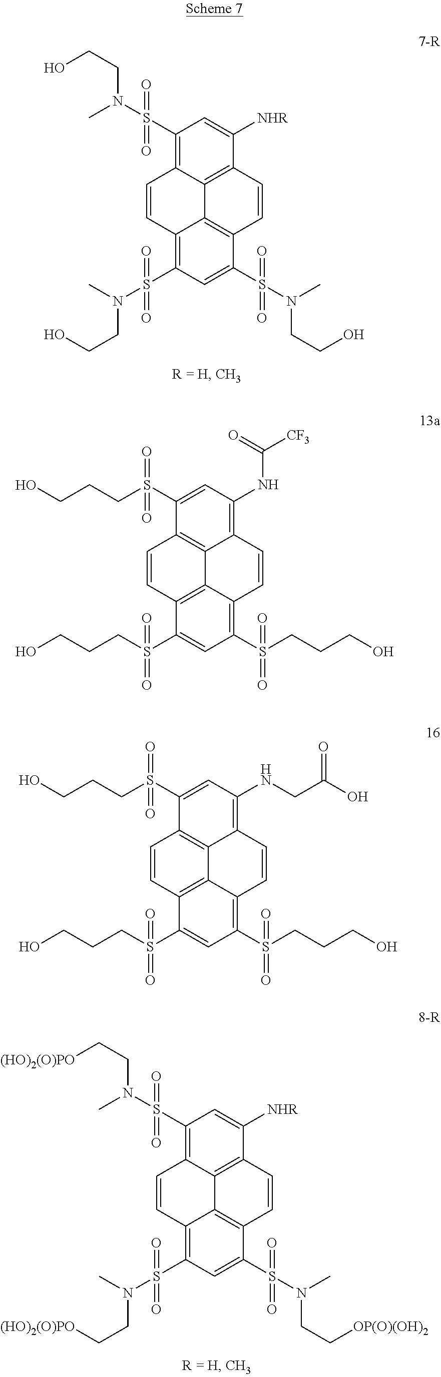

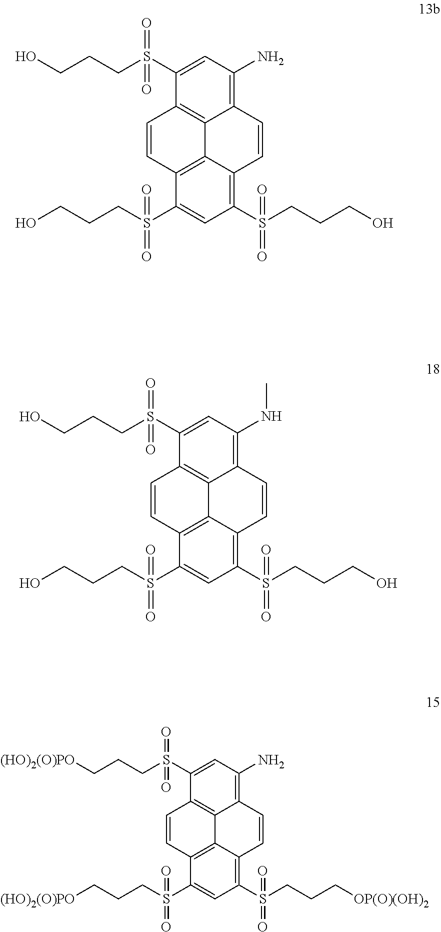

Exemplary aminopyrene-containing compounds of the invention and their precursors

Consequently, a closely related aspect of the present invention relate to the use of compounds of the structural Formulae A-D as fluorescent reagents for conjugation to a broad range of analytes, wherein the conjugation comprises formation of at least one covalent chemical bond or at least one molecular complex with a chemical entity or substance, such as amine, carboxylic acid, aldehyde, alcohol, aromatic compound, heterocycle, dye, amino acid, amino acid residue coupled to any chemical entity, peptide, protein, carbohydrate, nucleic acid, toxin and lipid.

The claimed compounds are suitable for and may be used in a method for fluorescent labelling and detecting of target molecules. Typically, such a method implies reacting a compound according to any one of Formulae A-D above with a target molecule selected from the group comprising amino acids, peptides, proteins, including primary and secondary antibodies, single-domain antibodies, docetaxel, avidin, streptavidin and their modifications, aptamers, (modified) nucleotides, (modified) nucleic acids, toxins, lipids, carbohydrates, including 2-deoxy-2-aminoglucose and other 2-deoxy-2-aminoaminopyranosides, glycans, glucans, (modified) biotin (e.g., biocytin), and other small molecules (e.g., jasplakinolide and its modifications). The labeling is followed by separation, detection, quantification and/or isolation of the labeled fluorescent derivatives by means of chromatographic and/or electrokinetic techniques.

The present inventors found that chromatographic separation techniques (like reversed phase or hydrophilic interaction (U)HPLC, in all possible scales (from nano to analytical scale and bigger) and electrokinetic separation techniques (electrophoresis, gelelectrophoresis, capillary electrophoresis, capillary gelelectrophoresis or capillary electrochromatotgraphy)—all with fluorescence or laser induced fluorescence detection—are well suited for the described improved method for automated high performance profiling, identification and/or determination of carbohydrates and carbohydrate mixtures. In particular using multiplexed capillary gel electrophoresis with laser induced fluorescence detection (xCGE-LIF) allows a fast but robust and reliable analysis and identification of carbohydrates and/or carbohydrate mixture composition patterns (e.g.: glycosylation patterns of glycoproteins). The methods according to the present invention used in the context of glycoprotein analysis allow to visualize carbohydrate-mixture compositions (e.g.: glycan-pools of glycoproteins) including structural analysis of the carbohydrates while omitting highly expensive and complex equipment, like mass spectrometers or NMR-instruments. Due to its superior separation performance and efficiency compared to other separation techniques, capillary electrophoresis techniques, in particular, capillary gel electrophoresis are considered for complex carbohydrate separation before but said technique was not recommended in the art due to drawbacks which should allegedly provided when using said method, see e. g. Domann et al. or WO2006/114663. However, when applying the method according to the present invention, the technique of xCGE-LIF allows for sensitive and reliable determination and identification of carbohydrate structures in high performance. In particular, the use of a capillary DNA-sequencer, (e. g. 4-Capillary Sequencers: 3100-Avant Genetic Analyzer, 3130 Genetic Analyzer, SeqStudio and Spectrum Compact; 16-Capillary Sequencer: 3100 Genetic Analyzer and 3130xl Genetic Analyzer; 48-Capillary Sequencer: 3730 DNA Analyzer; 96-Capillary Sequencer: 3730xl DNA Analyzer from Applied Biosystems, 8-Capillary Sequencers: 3500 Genetic Analyser; 24-Capillary Sequencers: 3500xl Genetic Analyser and Promega Spectrum) allows the high performance of the method according to the present invention. The advanced/improved method of the invention enables an easier and more precise characterization of variations in complex composed natural or synthetic carbohydrate mixtures and the characterization of carbohydrate mixture composition patterns (e.g.: protein glycosylation patterns), directly by carbohydrate “fingerprint” alignment in case of comparing samples with known carbohydrate mixture compositions.

The method according to the present invention is a further simplified and more robust but nevertheless highly sensitive and reproducible glycoanalysis method with high separation performance.

Especially the combination of the above mentioned instruments with up to 96 capillaries in parallel and the software/database tool enclosed within the invention, enables an automated real high throughput analysis.

A further specific embodiment of this aspect relates to a method for fluorescent labeling of carbohydrates with dyes of Formulae A-D comprises at least the following steps:

a) preparing a 1-400 mM solution of the dye, in particular a dye of the formula 6-H, 6-Me, 8-H, 15, 23 or 23b as shown in claim 5, in 0.5-4 M aqueous organic acid;
b) preparing a 0.05-3 M borane solution in DMSO, water, methanol, ethanol, diglyme, tetrahydrofurane or a mixture of these solvents;
c) mixing the solutions prepared in steps a) and b) above and a carbohydrate-containing analyte solution in a reaction vessel;
d) incubating the reaction mixture at 10-90° C. for 0.1-48 h;
e) adding a mixture of water and an organic solvent miscible with water, with a ratio of organic solvent: water in the range from 1:10 to 10:1, to the reaction mixture and agitating the contents of the reaction vessel, in order to stop the reaction in step d) and dissolve the reaction products;
f) optionally subjecting the mixture resulting from step e) to vortexing; and
g) optionally subjecting the mixture resulting from step f) to electrophoresis.

More specifically, the organic solvent is selected from the group comprising acetonitrile, ethanol, methanol, isopropanol, tetrahydrofurane, acetic acid, dioxane, sulfolane, dimethylsulfoxide, dimethylformamide, N-methylpyrrolidone, nitromethane, hexamethylphosphortriamide, diglyme, methyl cellosolve, and preferably the organic solvent is acetonitrile.

Further the present invention encompasses also carbohydrate-dye conjugates comprising a fluorescent dye according to Formulae A-B or A-D above.

More specifically, the dye in said conjugates, in particular carbohydrate-conjugates, is selected from the compounds of the formulae 6-H, 6-Me, 8-H, 15, 23, 23b as shown in Scheme 8 below.

Due to their reaktive group (aromatic amino (NH₂), hydrazine (NRNH₂), hydrazide (CONRNH₂), hydroxylamine (NROH), reactive carbamate (NHCOOR) or alkoxyamino (RONH₂), the compounds of Formulae A to D above are suitable and advantageous for the use in the reductive amination or direct condensation reaction with suited carbohydrates possessing an aldehyde group in a free form or protected form, e.g. as semiacetal, or an amino group (as shown in Schemes 2-6 and 8).

Consequently, closely related aspects of the present invention relate to this use and to a method for the reductive amination or direct condensation comprising reacting a compound of Formulae A-D above with a suited carbohydrate possessing an aldehyde group in a free form or as semiacetal, or an amino group, for a sufficient time to effect the reductive amination and chromatographic or electrokinetic separation of the labeled fluorescent derivatives optionally followed by detection of analytes by means of optical spectroscopy, including fluorescence detection and/or mass spectrometric detection. Examples of dye-conjugate structures are given in Scheme 8.

The compounds of Formulae A-D and the carbohydrate-dye conjugates comprising the same are especially suitable and advantageous for use in the spectral calibration of a fluorescence detector, in particular a detector for detection of laser induced fluorescence (LIF) as they are commonly used in C(G)E-systems.

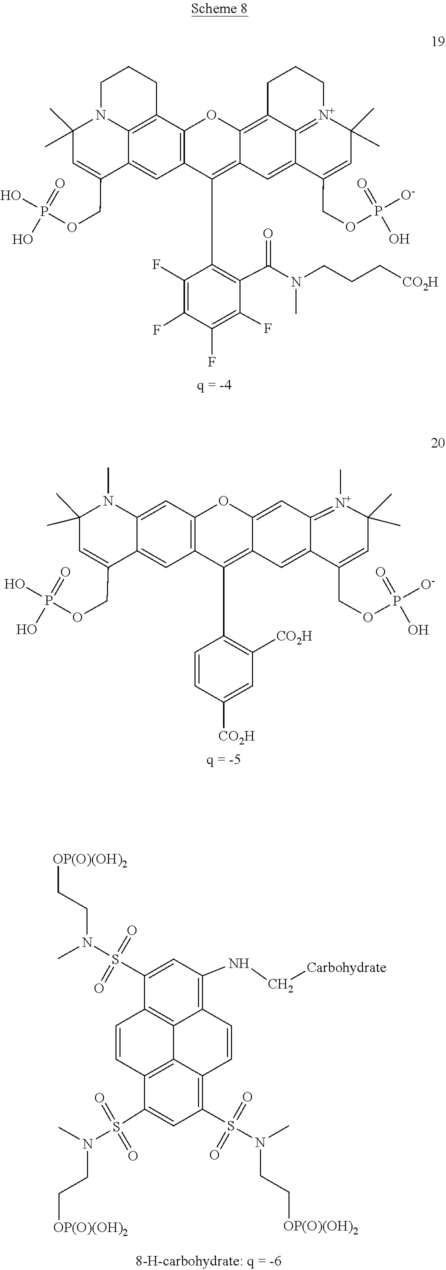

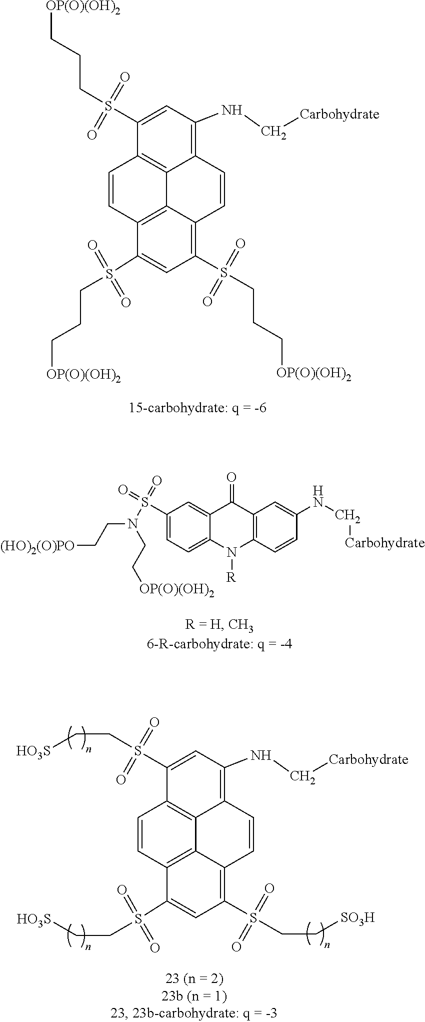

### Spectral Properties of the New Dyes

The spectral properties of the dyes are given in Table 1 below.

Table 1. Spectral properties of the phosphorylated aminoacridones 6-H and 6-Me, sulfonylamidopyrenes 8-R (R=H, Me), alkylsulfonyl-modified pyrene dyes 15, 16, 18, 23, as well as their precursors and related compounds: 19, 20 and dye APTS (see Schemes 7-13 for structures).

| | Absorption, λₘₐₓ, nm | Emission λₘₐₓ, nm | |
|---|---|---|---|
| Dye | (ε, M⁻¹ cm⁻¹) | (ϕₙ^{a}) | Solvent |
| 6-H | 217 (13500), 260 (26000) | 485 (excit. 405 nm), | H₂O |
| | 295 (28000), 420 (3700) | 586 (all excit. λ; ~0.05) | |
| 6-Me | 219 (10300), 263 (18600) | 485 and 585 | TEAB^{b} |
| | 299 (18500), 430 (2900) | (excit. 300-470 nm, | |
| | | ~0.06) | |
| 7-H | 477 (22400) | 535 (0.96)^{a} | MeOH |
| 7-Me | 493 (23000) | 549 (0.97) | MeOH |
| 8-H | 465 — | 544 (0.88) | H₂O |
| 8-Me | 502 — | 563 (0.85) | H₂O |
| 13b | 486 (21000) | 534 (0.80)^{c,d} | MeOH |
| 15 | 477 (19600) | 542 (0.92)^{g} | TEAB^{b} |
| 16 | 499 (18000) | 553 (0.71)^{d} | MeOH |
| 18 | 502 (23400) | 550 (0.88) | MeOH^{f} |
| | 509 (19500) | 563 (0.67) | H₂O^{f} |
| APTS^{e} | 425 (22000) | 457 (0.95)^{g} | PBS |
| 19 | 635 (75000) | 655 (0.62) | PBS |
| 20 | 581 (120000) | 607 (0.74) | PBS |
| 23 | 486 (21000) | 542 (0.86)^{g} | TEAB^{h} |

^{a}absolute values of the fluorescence quantum yields (if not stated otherwise);
^{b}TEAB is aqueous Et₃N*H₂CO₃ buffer with pH = 8-8.5;
^{c}excitation at 375 nm;
^{d}relative value, with Rhodamine 6G as a reference dye with ϕ_{fl} = 0.9;
^{e}for mono N-alkylated APTS derivatives abs. and emiss. maxima are 457 and 516 nm, respectively (ε~19000 M⁻¹ cm⁻¹);
^{f}excitation at 515 nm in aq. PBS buffer;
^{g}obtained with fluorescein as a reference dye with ϕ_{fl} = 0.9 in 0.1M NaOH under excitation at 496 nm;
^{h}none of the aminopyrene dyes including APTS showed significant changes while switching from PBS (pH 7.4) to TEAB buffer (pH 8-8.5).

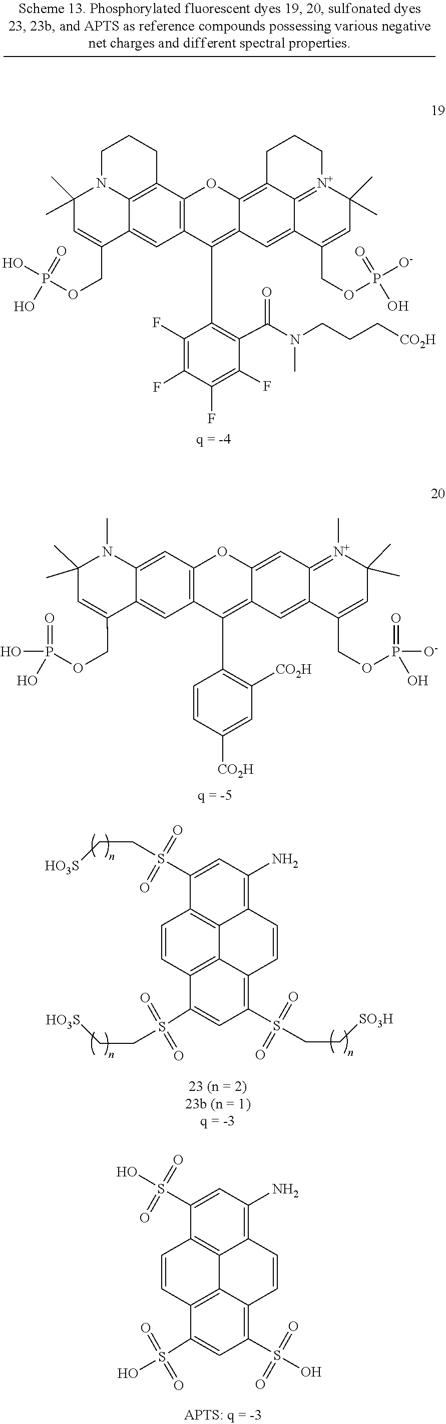

The structural features and data in Table 1demonstrate that the doubly phosphorylated aminoacridones 6-H and 6-Me, triple phosphorylated pyrene dyes 8-H, 8-Me, and 15 meet the criteria to the fluorescent tags defined above. Additionally, it was necessary to prove if they could be used in reductive amination of glycans, and if the emission of their conjugates would not interfere with the emission of glycans labeled with APTS (for structure and spectral data, see Scheme 7-12 and Table 1. For example, compounds 6-R (R=H, Me) have m/z ratios equal to 134 and 138, respectively (APTS has m/z=151). They have several absorption maxima and emit orange light (with two emission maxima at 485 nm and 585 nm and relative intensities of ca. 1:2; see FIG. 22A). Though their absorption at 488 nm is relatively low, the red-emission is a remarkable feature and corresponds to a Stokes shift of ca. 160 nm. The absolute values of the fluorescence quantum yields for compounds 6-R are 5-6%. Therefore, in spite of the relatively low brightness, even red-emitting dyes 6-R (pyrene dyes 8-R and 15 are brighter) represent new tags which can either be used for labelling of glycans, including “heavy” and “exotic” glycans which could not yet been detected due to limitations posed by APTS with its relatively low net charge (−3) and low mobility of the “heavy” carbohydrates decorated with an APTS label. Indeed, due to the presence of four negative charges and extremely low m/z ratio, phosphorylated dyes introduced here are able to provide better electrophoretic mobility of conjugates, reduce their migration times and thus reveal and highlight bulky and massive carbohydrates.

All pyrene dyes listed in Table 1 are highly fluorescent. The non-phosphorylated pyrenes 7-R (R=H, Me), 13b, 16 and 18 allow to estimate the extinction coefficients with higher accuracy. The extinction coefficients of the most long-wavelength bands are in the range of 18 000-23000, while the positions of the maxima vary from 465 to 507 nm. Therefore, the fluorescence can be readily induced by the argon ion laser emitting at 488 nm. Emission maxima are found in the range from 535 to 563 nm, and the fluorescence quantum yields are always high (71-97%). Therefore, sulfonated 1-aminopyrenes represent much brighter dyes than 2-sulfonamido-7-aminoacridones. The brightness is proportional to the product of the extinction coefficient (at 488 nm) and fluorescence quantum yield. We can assume that for acridone dyes this value is ca. 1500×0.06=90, and for pyrenes—20000×0.9=18000. This rough estimation means that trisulfonated 1-aminopyrenes are ca. 200 times brighter dyes than 2-sulfonamido-7-aminoacridones. This property makes pyrene dyes of the present invention to be superior tags than 2-sulfonamido-7-aminoacridones and APTS. If one assumes that for APTS conjugates the extinction coefficient at the maximum (457 nm) is 19000 (Scheme 6), and the absorption at 488 nm is typically ca. 35% of the maximal absorption at 457 nm, then one obtains the relative brightness of 6000 (assuming the same fluorescence quantum yield). Therefore, the dyes of the present invention are ca. 3 times brighter than APTS (in conjugates with glycans). Pyrene dyes of the present invention, in particular, compounds 8-H, 15, 23 and 23b represent new tags which can be used for labelling of glycans, including “heavy” and “exotic” glycans which could not yet been detected due to limitations posed by APTS its relatively low net charge (−3) and low brightness.

In order to shift the emission band to the red spectral region the N-methylated derivative 8-Me was prepared. This dye possesses a N-methylamino group and therefore, it represents a fluorophore which is very similar to the product of the reductive amination formed from glycans and the parent dye 8-H (compare with compound 6 in Scheme 9). The absorption maximum has been shifted to the red (+37 nm; 8-H→8-Me), but the emission maximum underwent the bathofluoric shift of “only” 19 nm (see Table 1). Thus, the Stokes shift reduced from 79 nm to 61 nm.

There is another tool for increasing bathochromic and bathofluoric shifts in the series of aromatic fluorescent dyes, provided that they possess electron-donor and electron-acceptor groups having the so-called “push-pull” electronic interactions between them (direct polar conjugation). In the case of 1-aminopyrene dyes, the donor group is fixed (and its electron donating properties cannot be enhanced), but the electron-withdrawing groups in positions 3, 6 and 8 may be varied. Particularly, the alkyl sulfone groups (R—SO₂, present in compounds 13b, 15, 16, 18, 23 and 23b) proved to be even more powerful acceptors than sulfonamide moieties (that are present in compounds 7-H, 7-Me, 8-H, 8-Me; see Scheme 7). However, after preparing compounds 8-H and 15 and comparing their spectral properties in aqueous solutions (Table 1), it was determined that, as expected, the bathochromic shift was 12 nm, but the position of the emission maximum and the band form were unchanged. The simplest explanation for that is based on the assumption that the single amino group (as a donor) is “at its limit” and not capable to provide more electron density to the π-system decorated with three very powerful acceptor groups, however strong they are. Fortunately, upon the reductive alkylation of the nitrogen atom (see Scheme 2), further bathochromic and bathofluoric shifts occurred (compare the spectral data for compounds 8-H and 8-Me discussed above), and compound 15 afforded bright conjugates with glycans featuring no cross-talk with APTS detection channel.

The invention is based on separating and detecting said carbohydrate mixtures (e.g.: glycan pools) utilizing the xCGE-LIF technique, e.g. using a capillary DNA-sequencer which enables generation of carbohydrate composition pattern fingerprints, the automatic structure analysis of the separated carbohydrates via database matching of the internally normalized CGE-migration time of each single compound of the test sample mixture. The method claimed herein allows carbohydrate mixture composition profiling of synthetic or natural sources, like glycosylation pattern profiling of glycoproteins. The advanced internal normalization of the migration times of the carbohydrates to migration time indices is based on the usage of sets of internal carbohydrate standards similar to the samples but labelled with (a) novel fluorescent dye(s) with an emission at another wavelength than the samples label(s). Said internal carbohydrate standards of known composition, e.g. can be a set of mono-, di- tritetra- and/or pentamers linear and/or branched up to 100mers (or higher)), eluting/migrating throughout of the whole range of the fingerprint of the carbohydrate samples to be analyzed, but being detected in another trace/channel, as they are fluorescently labelled with another tag than the carbohydrate samples and thus are emitting at another wavelength and don't show up in the samples trace. This advanced internal carbohydrate standards, eluting/migrating throughout of the whole migration/retention time range of the fingerprints of the carbohydrate samples to be analyzed, but being detected in another wavelength trace can be used for a very precise and reproducible “advanced” internal normalization of migration/retention times. They are used for the generation of the calibration curve, very precise regarding its curvature/form, y-axis intercept and its slope.

This improved determining of migration time indices allows an extremely exact and absolute reproducible analysis of carbohydrates, independent from sample type and origin, time-point of analysis, laboratory, instrument and operator.

The use of said method in combination with the system also allows to analyze said carbohydrate mixture compositions quantitatively. Thus, the method according to the present invention as well as the system represents a powerful tool for monitoring variations in the carbohydrate mixture composition like the glycosylation pattern of proteins without requiring complex structural investigations. For fluorescently labelled carbohydrates, the LIF-detection allows a limit of detection down to the attomolar range.

The standard necessary for alignment of each run may be present in a separate sample or may be contained in the carbohydrate sample to be analysed.

One of the fluorescent label used for labelling the carbohydrates may be e.g. the fluorescent labels 8-amino-1,3,6-pyrenetrisulfonic acid also referred to as 9-aminopyrene-1,4,6-trisulfonic acid (APTS) or other preferably multiple charged fluorescent dyes while the other fluorescent label is one of the dyes of the general Formula A or B.

Based on the presence of the standard, qualitative and quantitative analysis can be effected. Relative quantification can be done easily just via the individual peak heights of each compound, which corresponds linear (within the linear dynamic range of the LIF-detector) to its concentration.

The present invention resolves drawbacks of other methods known in carbohydrate analysis, like chromatography, mass spectrometry and NMR. NMR and mass spectrometry represent methods which are time and labour consuming technologies. In addition, expensive instruments are required to conduct said methods. Further, most of said methods are not able to be scaled up to high-throughput methods, like NMR techniques. Using mass spectrometry allows a high sensitivity. However, configuration can be difficult and only unspecific structural information could be obtained with addressing linkages of monomeric sugar compounds. HPLC is also quite sensitive depending on the detector and allows quantification as well. But as mentioned above, real high throughput analyses are only possible with an expensive massive employment of HPLC-Systems and solvents.

Other techniques known in the art are based on enzymatic treatment which can be very sensitive and result in detailed structure information, but require a combination with other methods like HPLC, MS and NMR. Further techniques known in the art relates to lectin or monoclonal antibody affinity providing only preliminary data without given definitive structural information.

The methods according to the present invention allow for high-throughput identification of carbohydrates mixtures having unknown composition or for high-throughput identification or profiling of carbohydrate mixture composition patterns (e.g.: glycosylation patterns of glycoproteins). In particular, the present invention allows determining the components of the carbohydrate mixture composition quantitatively.

The method of the present invention enables the fast and reliable measurement even of complex mixture compositions, and therefore enables determining and/or identifying the carbohydrates and/or carbohydrate mixture composition patterns (e.g.: glycosylation pattern) independent of the apparatus used but relates to the aligned migration times (migration time indices) only.

The invention allows for application in diverse fields. For example, the method maybe used for analysing the glycosylation of mammalian cell culture derived molecules, e.g. recombinant proteins, antibodies or virus or virus components, e.g. influenza A virus glycoproteins. Information on glycosylation patterns of said compounds are of particular importance for food and pharmaceuticals. Starting with the separation of complex protein mixtures by 1 D/2D-gel-electrophoresis, the method of the present invention could be used also for glycan analysis of any other glycoconjugates.

Moreover, pre-purified glycoproteins, e.g. by chromatography or affinity capturing, can be handled as well as by the method according to the present invention, substituting the gel separation and in-gel-degylcosylation step with in-solution-deglycosylation, continuing after protein and enzyme precipitation. Finally, complex soluble oligomeric and/or polymeric saccharide mixtures, obtain synthetically or from natural sources which are nowadays important nutrition additives/surrogates or as used in or as pharmaceuticals can be analysed.

Thus, two types of analyses may be performed on the carbohydrate mixtures. On the one hand, carbohydrate mixture composition pattern profiling like glycosylation pattern profiling may be performed and, on the other hand, carbohydrate identification based on matching carbohydrate migration time indices with data from a database is possible.

Therefore, a wide range of potential applications for the method according to the present invention is given ranging from production and/or quality control to early diagnosis of diseases which are producing, are causing or are caused by changes in the glycosylation patterns of glycoproteins.

In particular, in medical diagnosis, e.g. chronic inflammation recognition or early cancer diagnostics, where changes in the glycosylation patterns of proteins are strong indicators for disease, the method may be applied. The variations in the glycosylation pattern could simply be identified by comparing the obtained fingerprints regarding peak numbers, heights and migration times. Thus, disease markers may be identified, as it is described in similar proteomic approaches. It is, similar to comparing the proteomes of an individual at consecutive time points, the glycome of individuals could be analysed as indicator for disease or identification of risk patients.

In an embodiment, the method according to the present invention is a method wherein the fluorescent dye is a dye having the following Formula C

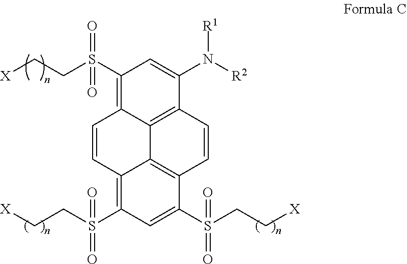

In another embodiment, the fluorescent dye is a dye having the formula of Formula D

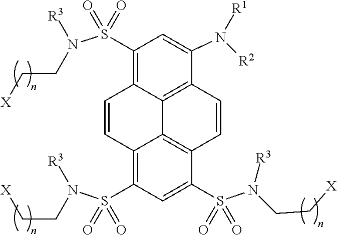

In a preferred embodiment, the compounds of Formulae A to D are selected from

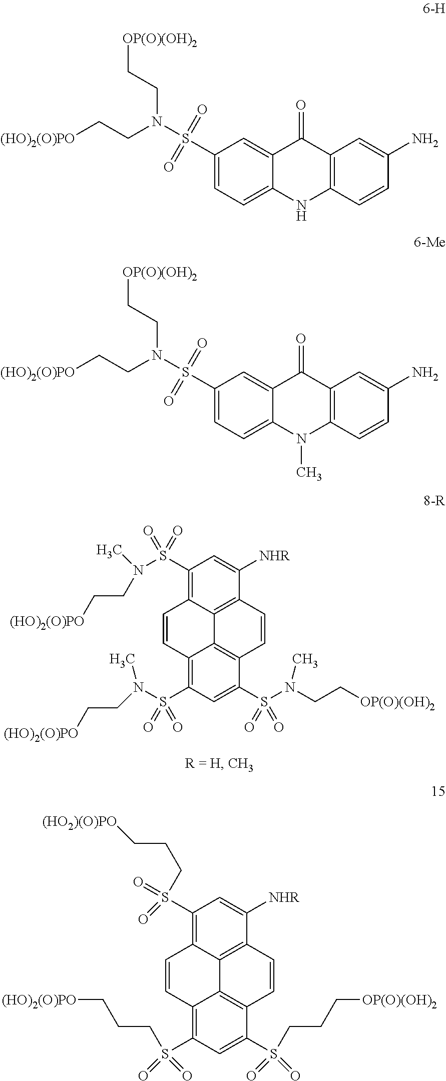

or a compound of 7-R (R=H, Me), 13a, 13b, 16 and 18

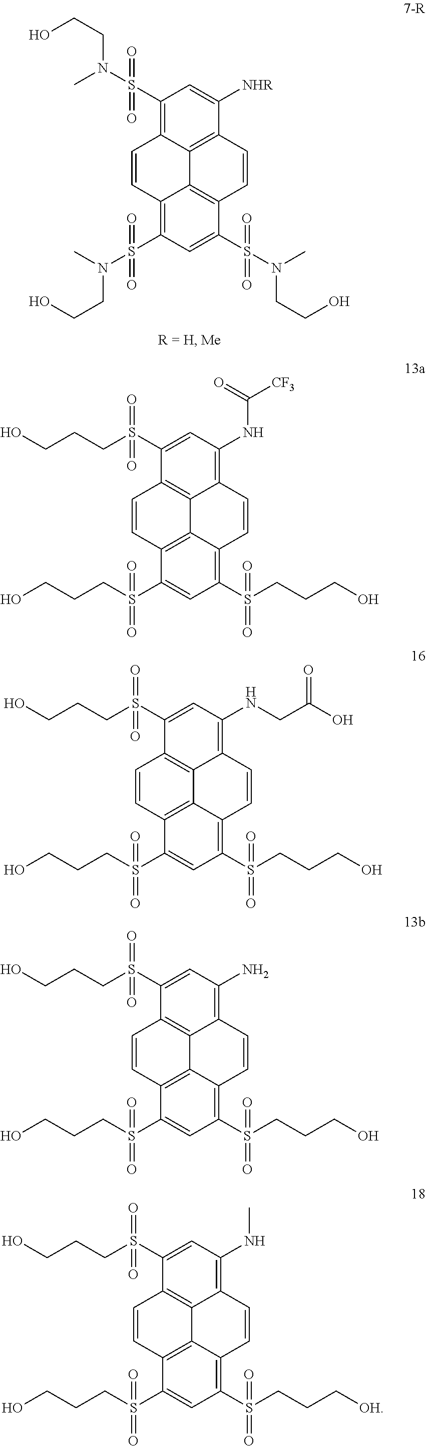

In another aspect, the present invention relates to a method for calibration of a multi wavelength fluorescence detection system, in particular, a capillary gel electrophoresis system, with acridone and/or pyrene based fluorescent dyes, which may optionally be present as conjugates with a substrate moiety including carbohydrates, whereby the method includes the detection of at least one of the compounds according to Formula A or B as defined in claim 1, including compounds C or D, together with additional fluorescent dyes admitting at different wavelength, preferably including at least one of the compounds APTS, compound 19 or compound 20 as shown in the following

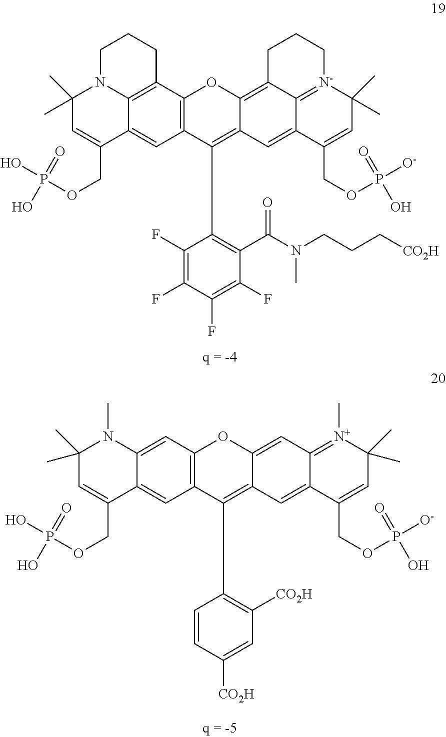

As demonstrated in the examples, the calibration of the multi wavelength fluorescence detection system with the dyes as described increase the sensitivity of the instrument and allows to conduct the methods according to the present invention more independently from the operator, the instruments, etc.

In particular, as discussed in the examples further, calibration of the system or instrument increase sensitivity and thus, suitability and usability of the methods as described.

In an embodiment of the method for calibration according to the present invention, the acridone and/or pyrene based dyes and there combinations utilized for the spectral calibration are shown in Table 2 and Table 3 inside Example 2, respectively Example 3.

Moreover, according to the present invention a carbohydrate dye conjugate comprising fluorescent dyes according to the present invention for use in a method according to the present invention is disclosed. In an embodiment, the dye conjugate according to the present invention is a dye selected from the compounds of the formula below

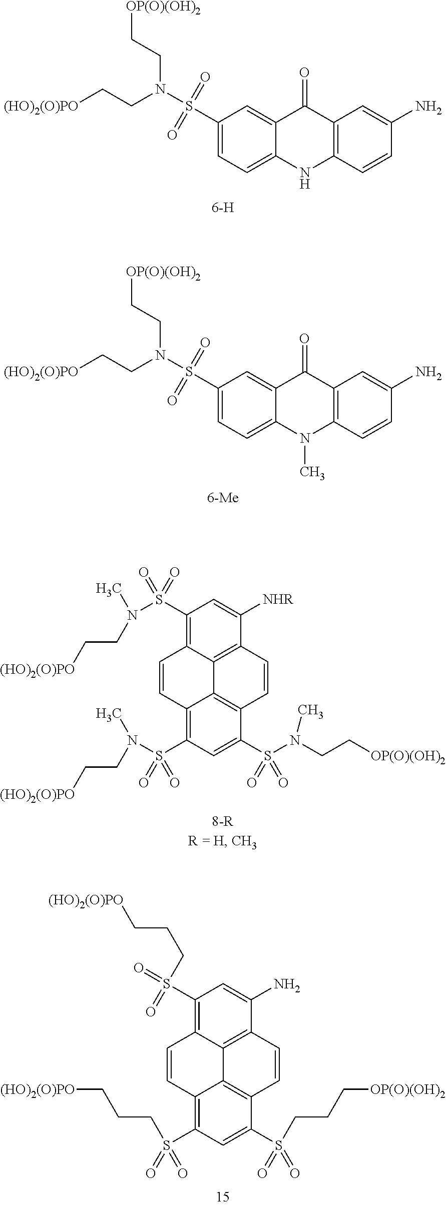

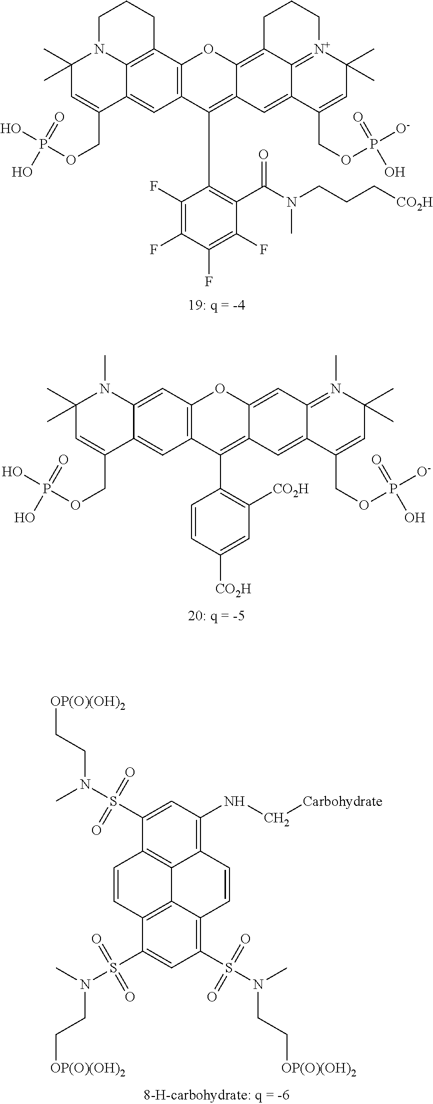

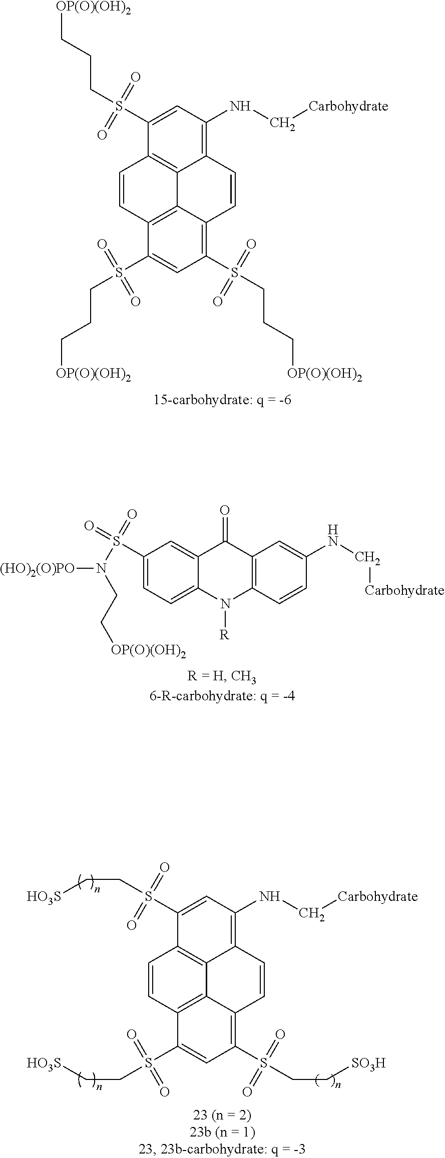

In a further aspect, a calibration standard is provided. Namely, the calibration standard useful e.g. in the method for calibration as described herein is a carbohydrate standard including a fluorescence dye including at least one of a fluorescence dye according to Formula A, B, C or D, which may be conjugated with a carbohydrate, optionally further comprising at least one of compounds 19 or 20.

Typical examples of the calibration standard are described in connection with the method for wavelength calibration.

In another aspect, the present invention relates to standard composition composed of compounds labelled with a fluorescence dye according to Formula A or B, in particular, of Formula C or D or different dyes of Formulae A to D. In an embodiment, the standard composition is composed of carbohydrates labelled with said dye, alternatively, the compounds are a DNA base pair ladder or similar nucleic acid base standards. Further, the dyes are preferably at least one of 6-H, 6-Me, 8-R, 15, 13a, 13b, 16, 18, 23 and 23b. Said standard composition is useful in a method according to the present invention, in particular, the alignment of the migration/retention times of the carbohydrates to be determined.

Further, the compound of Formula 20 is disclosed.

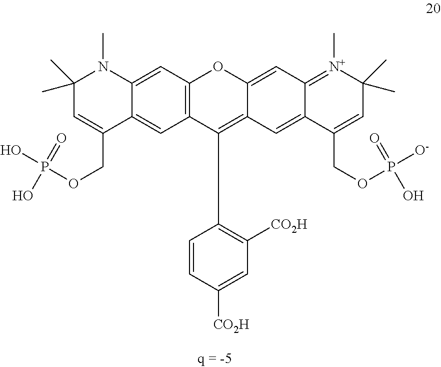 [SMILES: CN1c2cc3c(cc2C(COP(=O)(O)O)=CC1(C)C)C(c1ccc(C(=O)O)cc1C(=O)O)=c1cc2c(cc1O3)=[N+](C)C(C)(C)C=C2COP(=O)([O-])O]

In a further aspect, the present invention relates to a kit or system for determining and/or identifying carbohydrate mixture composition patterns comprising a data processing unit having a non-transient memory, said memory containing a database, said database containing aligned migration/retention times and/or aligned migration/retention time indices of carbohydrates, said migration/retention times and/or migration/retention time indices are obtained by an automated determination and/or identification of carbohydrates and/or identification of carbohydrates and/or carbohydrate mixture composition pattern profiling comprising the steps of:

a) obtaining a sample containing at least one carbohydrate;
b) labelling said carbohydrate(s) with a first fluorescent label;
c) providing a standard of known composition labelled with a second fluorescent label;
d) determining the migration/retention time(s) of said carbohydrate(s) and the standard of known composition as described herein, e.g. using capillary gel electrophoresis-laser induced fluorescence;
e) aligning the migration/retention time(s) to migration/retention time indice(s) based on given standard migration/retention time indice(s) of the standard;
f) comparing these migration/retention time indice(s) of the carbohydrate(s) with standard migration/retention time indice(s) from a database;
g) identifying or determining the carbohydrate(s) and/or the carbohydrate mixture composition pattern,
wherein the standard composition is added to the sample containing the unknown carbohydrate mixture composition, the first fluorescent label and the second fluorescent label are different and wherein the first fluorescent label or the second fluorescent label is a fluorescent dye having multiple ionizable and/or negatively charged groups which is selected from the group consisting of compounds of the general Formulae A to D.

In another aspect, the present invention relates to a kit or system for determining and/or identifying carbohydrate mixture composition pattern profiling comprising a data processing unit having a non-transient memory, said memory containing a database, said database containing aligned migration/retention times and/or aligned migration/retention time indices of carbohydrates, said migration/retention times and/or migration/retention time indices are obtained by an automated determination and/or identification of carbohydrates and/or identification of carbohydrates and/or carbohydrate mixture composition pattern profiling comprising the steps of

a) providing a sample containing a carbohydrate mixture composition;
b) labelling of said carbohydrate mixture composition with a first fluorescent label;
c) providing a second sample labelled with a fluorescent label having a known carbohydrate mixture composition pattern to be compared with;
d) generating electropherograms/chromatograms of the carbohydrate mixture composition of the first and second sample as described in a method disclosed herein, e.g. using capillary (gel) electrophoresis-laser induced fluorescence or chromatography;
e) comparing the standard migration/retention time indices calculated from the obtained electropherogram/chromatogram of the first sample and the second sample;
f) analyzing the identify and/or differences between the carbohydrate mixture composition pattern profiles of the first and second sample, wherein standard migration/retention time indices of the carbohydrates present in the sample are calculated based on internal standards of known composition labelled with a second fluorescent label and wherein one of the first or second fluorescent label is a fluorescent dye according to the present invention of general Formula A or B.

Moreover the present invention relates in a further aspect to a kit or system for an automated carbohydrate mixture composition pattern profiling comprising a data processing unit having a non-transient memory, said memory containing a database, said database containing aligned migration/retention times and/or aligned migration/retention time indices of carbohydrates, said migration times and/or migration/retention time indices are obtained by an automated determination and/or identification of carbohydrates and/or identification of carbohydrates and/or carbohydrate mixture composition pattern profiling comprising the steps of

a) providing a first sample containing an unknown carbohydrate mixture composition;
b) labelling of said carbohydrate mixture composition with a first fluorescent label;
c) adding a second sample having a known carbohydrate mixture composition pattern labelled with a second fluorescent label to said first sample;
d) generating electropherograms/chromatograms of the carbohydrate mixture composition of said sample using capillary (gel) electrophoresis-laser induced fluorescence or chromatography;
e) analyzing the identity and/or differences between the carbohydrate mixture composition pattern profiles of the first and the second sample, wherein the first fluorescent label of the first sample is different to the second fluorescent label of the second sample and wherein at least one of the first fluorescent label and the second fluorescent label is a fluorescent dye according to general Formula A or B according to the present invention.

In an embodiment, the kit or system according to the present invention comprises further a capillary (gel) electrophoresis-laser induced fluorescence apparatus. For example, this apparatus may be a capillary DNA-sequencer known in the art.

In a further aspect, a carbohydrate dye conjugate comprising the fluorescent dyes as defined herein conjugated with carbohydrates as described herein for use in a method according to the present invention is disclosed.

An embodiment, the carbohydrate dye conjugate is a conjugate wherein the dye is selected from the compounds of the following formula:

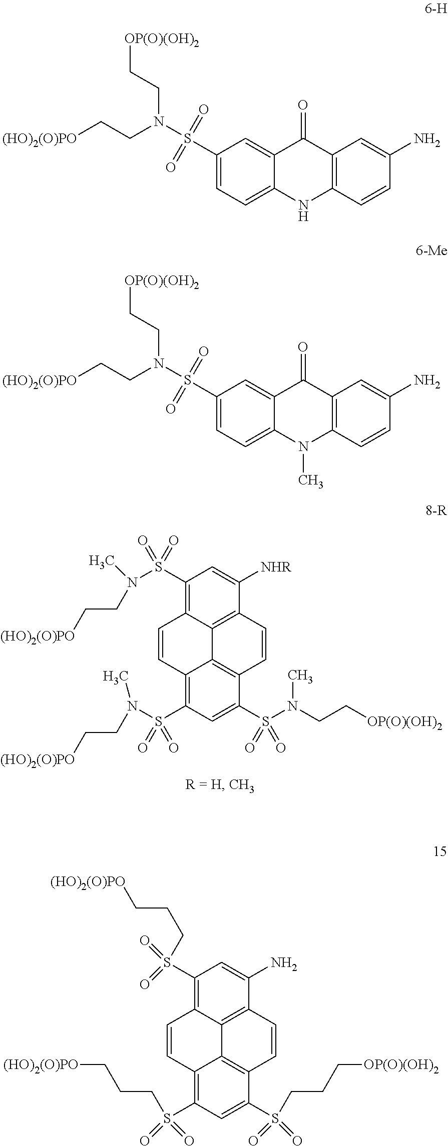

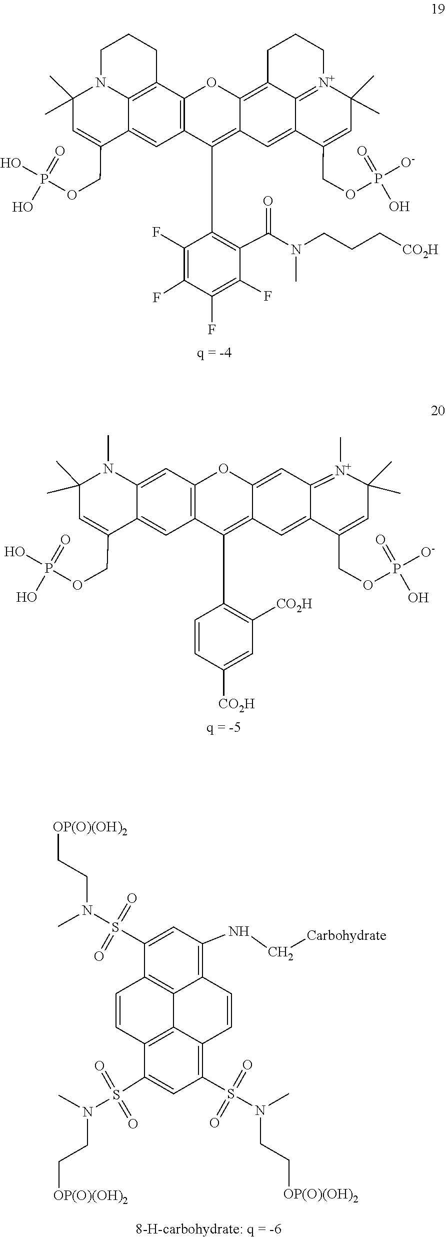

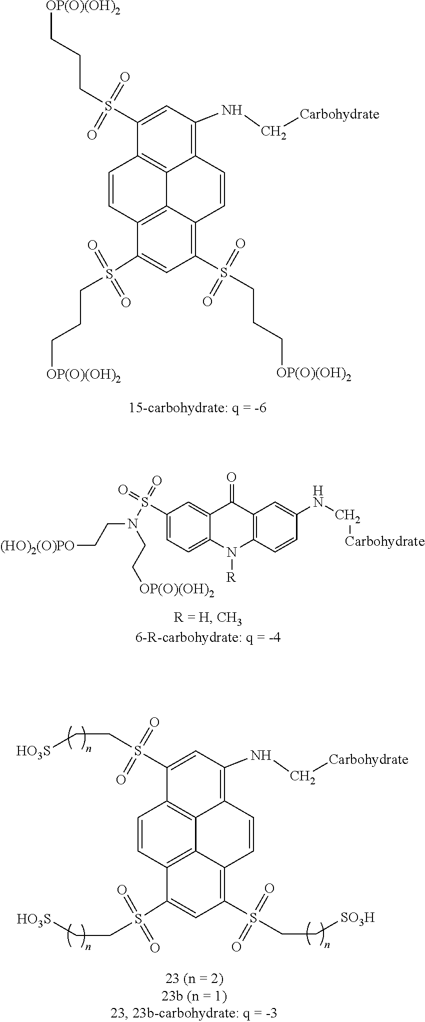

In some embodiments of the specific compounds mentioned above, the dyes are present as a carbohydrate dye conjugate identifying the carbohydrate bound to the dye accordingly.

The invention will be described further by way of examples illustrating the present invention in more detail without limiting the same thereto.

### BRIEF DESCRIPTION OF THE FIGURES

FIG. 1—provides a workflow of the carbohydrate analysis according to the present invention.

FIG. 2—Spectral calibration mixture of 19 (I), 20 (II), 6-H-labeled maltotriose (6-H^{a}; III) and APTS-labeled maltotetraose (APTS^{a}; IV) before (A) and after (B) spectral calibration of the xCGE-LIF instrument to the particular calibration mixture of these four dyes.

FIG. 3—6-H labeled maltose ladder before (A) and after (B) spectral calibration of the xCGE-LIF instrument to 19, 20, 6-H^{a} and APTS^{a}. VB9163 labeled maltose ladder in B was 1:2 diluted in water before measurement. Peaks depicted are maltose at 13.2 min, maltotriose at 15.3 min, maltotetraose at 17.2 min, maltopentaose at 19 min, maltohexaose at 20.8 min, maltoheptaose at 22.2 min, maltooctaose at 23.9 min and so on.

FIG. 4—Spectral calibration mixture of 15-labeled maltotriose (15^{a}; I), 19 (1), 20 (IV), 6-Me-labeled maltotriose (6-Me^{a}; V) and APTS-labeled maltotetraose (APTS^{a}) before (A) and after (B) spectral calibration of the xCGE-LIF instrument to the particular calibration mixture of five dyes.

FIG. 5—APTS labeled dextran ladder (APTS^{b}) before (A) and after (B) spectral calibration of the xCGE-LIF instrument to 15^{a}, 19, 20, 6-Me^{a} and APTS^{a}. Peaks depicted are dextran-trimer at 14.1 min, -tetramer at 16.2 min, -pentamer at 18.3 min, -hexamer at 20.9 min, -heptamer at 23 min and so on.

FIG. 6—15-labeled dextran ladder (15^{b}) before (A) and after (B) spectral calibration of the xCGE-LIF instrument to 15^{a}, 19, 20, 6-Me^{a} and APTS^{a}. Peaks depicted are dextran-trimer at 9.8 min, -tetramer at 11 min, -pentamer at 12 min, -hexamer at 13.1 min. -heptamer at 14.2 min and so on.

FIG. 7—6-Me-labeled dextran ladder (6-Me^{b}) before (A) and after (B) spectral calibration of the xCGE-LIF instrument to 15^{a}, 19, 20, 6-Me^{a} and APTS^{a}. Peaks depicted are dextran-trimer at 14.9 min, -tetramer at 16.3 min, -pentamer at 18.2 min, -hexamer at 20.1 min, -heptamer at 22 min and so on.

FIG. 8—Overlay of APTS labeled citrate plasma derived N-glycans (522 nm trace), 15 labeled carbohydrate standard (554 nm trace) and 6-Me labeled carbohydrate standard (575 nm trace) after spectral calibration of the xCGE-LIF instrument to 15^{a}, 19, 20, 6-Me^{a} and APTS^{a} (see FIG. 7). 522 nm, 554 nm and 575 nm channels shows now spectral crosstalk with other channels proving the successful spectral calibration.

FIG. 9—Electropherograms of different alignment standards. A—GeneScan 500 LIZ Size Standard. B—acridone based fluorescent dye (6-Me) labeled carbohydrate standard. Marked peaks were used to calculate the polynomial fit for the alignment procedure (see FIG. 11).

FIG. 10—Human citrate plasma derived N-glycan fingerprint after alignment to base pair size standard (A) or to base pair size standard refined by an orthogonal carbohydrate standard (B). The relative peak height proportion (PHP) is a signal intensity normalization of fingerprint to the sum of 15 picked peaks. Polymer 1 and 2 are of different production dates/batches. Day 1-9 counts the days the polymer was at room temperature.

FIG. 11—Human citrate plasma derived N-glycan fingerprint after alignment to base pair size standard (A) or an acridone fluorescent dye labeled carbohydrate standard (6-Me^{b}) (B). The relative peak height proportion (PHP) is a signal intensity normalization of fingerprint to the sum of 15 picked peaks. Polymer 1 and 2 is POP7 polymer of different production dates. Day 1-9 counts the days of POP7 polymer at room temperature.

FIG. 12—Polynomial fit of the internal standards for different alignment procedures. A—2^{nd} order polynomial fit for the alignment to base pair size standard. 13 peaks were picked as shown in FIG. 9 A. B—2^{nd} order polynomial fit for the alignment to base pair size standard, adjusted by a 2^{nd} alignment step, using four internal oligosaccharide peaks. C—2^{nd} order polynomial fit for the alignment to an acridone based fluorescent dye (6-Me) labeled carbohydrate standard. 16 peaks were picked as shown in FIG. 9 B.

FIG. 13—Electropherograms of different alignment standards. A—base pair size standard. B—pyrene based fluorescent dye (15) labeled carbohydrate standard. Marked peaks were used to calculate the polynomial fit for the alignment procedure (see FIG. 16).

FIG. 14—Human citrate plasma derived N-glycan fingerprint after alignment to base pair size standard (A), to base pair size standard+a pyrene fluorescent dye labeled carbohydrate standard (B), or a pyrene fluorescent dye (15) labeled carbohydrate standard (15^{b}) (C). The relative peak height proportion (PHP) is a signal intensity normalization of fingerprint to the sum of 15 picked peaks. Polymer 1 and 2 is POP7 polymer of different production dates. Day 1-9 counts the days of POP7 polymer at room temperature.

FIG. 15—Overlay of APTS labeled citrate plasma derived N-glycans (522 nm trace), 15-labeled carbohydrate standard (554 nm trace) and base pair standard (655 nm trace) after spectral calibration of the xCGE-LIF instrument to 15^{a}, 19, 20, 6-Me^{a} and APTS^{a} (see FIG. 7). 522 nm and 554 nm channel shows now spectral crosstalk with other channels proving the successful spectral calibration. A small spectral cross talk can be observed of the base pair size standard containing 655 nm channel with the 595 nm and 575 nm channel, as the 655 nm channel was not spectral calibrated to the bp dye.

FIG. 16—Polynomial fit of the internal standards for different alignment procedures. A—2^{nd} order polynomial fit for the alignment to base pair size standard. 13 peaks were picked as shown in FIG. 13 A. B—2^{nd} order polynomial fit for the alignment to an pyrene based fluorescent dye (15) labeled carbohydrate standard. 22 peaks were picked as shown in FIG. 13 B.

FIG. 17—Overlay of APTS labeled citrate plasma derived N-glycan fingerprints measured with different instruments and alignment to base pair size standard (A), base pair size standard+oligosaccharide re-alignment (B), base pair size standard+pyrene fluorescent dye (23) labeled carbohydrate standard re-alignment (C) or a pyrene fluorescent dye (23) labeled carbohydrate standard (D). With 3130_1—first ABI DNA Genetic Analyzer 3130 (serial number: 21363-yyy) equipped with a 50 cm four capillary array, 3130_2—second ABI DNA Genetic Analyzer 3130 (serial number: 1521-yyy) equipped with a 50 cm four capillary array, 3130xl_1—first ABI DNA Genetic Analyzer 3130xl (serial number: 19248-yyy) equipped with a 50 cm 16-capillary array, 3130xl_2—second ABI DNA Genetic Analyzer 3130xl (serial number: 1208-yyy) equipped with a 50 cm 16-capillary array, 3500—Thermo Scientific DNA Analyzer 3500 (serial number: 21106-yyy) equipped with a 50 cm eight-capillary array, 3730—ABI DNA Genetic Analyzer 3730 (serial number: 18124-yyy) equipped with a 50 cm 48-capillary array. All measurements were performed with POP7.

FIG. 18—Overlay of APTS labeled citrate plasma derived N-glycan fingerprints measured with different electric field strengths and alignment to base pair size standard (A) or a pyrene fluorescent dye (23) labeled carbohydrate standard (B). Measurements were performed with ABI DNA Genetic Analyzer equipped with a glyXpop_fast filled 50 cm capillary array with the field strength of 300 V/cm (“ [symbol] ” curve, 15 kV), 200 V/cm (“ [symbol] ” curve, 10 kV), or 100 V/cm (“-” curve, 5 kV).

FIG. 19—Overlay of APTS labeled citrate plasma derived N-glycan fingerprints measured at different run temperatures and alignment to base pair size standard (A) or a pyrene fluorescent dye (23) labeled carbohydrate standard (B). Measurements were performed with ABI DNA Genetic Analyzer equipped with a POP7 filled 50 cm capillary array and operated at a run temperatures of 45° C. (“ [symbol] ” curve), 30° C. (“ [symbol] ” curve), or 18° C. (“-” curve).

FIG. 20—Overlay of APTS labeled citrate plasma derived N-glycan fingerprints measured with different capillary array lengths and alignment to base pair size standard (A) or a pyrene fluorescent dye (23) labeled carbohydrate standard (B). Measurements were performed with ABI DNA Genetic Analyzer equipped with a POP7 filled 50 cm capillary array (“ [symbol] ” curve), 36 cm capillary array (“ [symbol] ” curve), or 22 cm capillary array (“-” curve).

FIG. 21—Overlay of APTS labeled citrate plasma derived N-glycan fingerprints measured with different separation polymers. Not aligned electropherogram are depicted in minutes (A), fingerprints alignment to base pair size standard are depicted in base pairs (B) and fingerprints aligned to a pyrene fluorescent dye (23) labeled carbohydrate standard are depicted in oligosaccharide units (C). Measurements were performed with ABI DNA Genetic Analyzer equipped with 50 cm capillary array and filled with POP7 (Thermo Scientific; black curve), nanoPOP7 (MCLAB; grey curve), nimaPOP7 (Nimagen; light grey curve), POP6 ((Thermo Scientific; black “ [symbol] ” curve), or glyXpop_fast (experimental polymer from glyXera GmbH; black “ [symbol] ” curve).

[symbol]

FIG. 22—Overlay of APTS labeled human IgG derived N-glycan fingerprints aligned to a pyrene fluorescent dye (23) labeled carbohydrate standard. Measurements were performed with ABI DNA Genetic Analyzer equipped with 50 cm capillary array and filled with POP7 polymer. Measurements were performed by re-injection of the same sample with the polymer age D1-D52 (counts the days of POP7 polymer at room temperature inside of the instrument).

FIG. 23 Emission spectra of the dyes used in DNA sequencing (one of the several possible sets is shown), and the corresponding set of virtual filters. 5-FAM: 5′-carboxy-fluorescein; JOE: 2,7-dimethoxy-3,4-dichlorofluorescein 6′-carboxy isomer; NED is a brighter dye than TMR (with unknown structure); it has absorption and emission maxima at 546 nm and 575 nm, respectively. ROX is rhodamine with two julolidine fragments incorporated into the xanthene fluorophore (and 5′- or 6′-carboxyl group). In the course of fluorescent sequencing, these (or similar) dyes provide four color traces; e.g., blue—for cytosine, green—for adenine, red—for thymine, and yellow—for guanine.

FIG. 24 A Shows the normalized absorption and emission spectra of phosphorylated aminoacridone dyes 6-H and 6-Me in aqueous triethyl amine—bicarbonate buffer (pH 8).

FIG. 24 B Shows the normalized absorption and emission spectra of the triphosphorylated aminopyrene dyes 8-H and 15 in aqueous triethyl amine—bicarbonate buffer (pH 8).

FIG. 25 Presents an overview of electropherograms of two dyes: tri-phosphorylated aminopyrene 8-H und APTS with an APTS-labeled maltose ladder (on the background). The retention time of 8-H is higher than the retention time of APTS, though the m/z ratio for 8-H (144) is lower that of APTS (151). In APTS, the charged groups (sulfonic acid residues) are directly attached to fluorophore. The presence of N-methyl-N-(2-hydroxyethyl) linker in 8-H increases the hydrodynamic ratio of the dye, and this explains higher retention time of the free dye 8-H.

FIG. 26 Displays the zoomed peaks of 8-H und APTS. This figure was obtained with a color calibration of a standard DNA sequencer. The five color channels of the “traditional” filter sets are present: 522 nm (fluorescein, APTS), 554 nm (e.g., VIC dye or Rhodamine 6G), 575 nm (e.g, NED dye or TMR), 595 nm (e.g., PET dye or ROX), and 650 nm (LIZ dye as an additional, “fifth” color). Do to the strong cross-talk with an APTS color channel (shown in upper part of the figure), dye 8-H (and probably its conjugates with glycans) cannot be used together with APTS in any analytical assays. The same is true for the tri-phosphorylated pyrene dye 15 (compare the emission spectra of 8-H and 15 shown in FIG. 24 B). Therefore, a new color calibration of the DNA sequencer was necessary, in order to reduce or, if possible, fully eliminate cross-talk between the emission channels attributed to APTS and tri-phosphorylated pyrene dyes 8-H and 15.

FIG. 27 Shows an electropherogram of the reductive amination product obtained from maltotriose and dye 15 (15^{a}) before spectral calibration.

FIG. 28 Show the same electropherogram (FIG. 27) of the reductive amination product obtained from maltotriose and dye 15 after spectral calibration.

FIGS. 29A and B Shows the electropherograms of the conjugates obtained from the mixtures of carbohydrates “dextran 1000” (29 A) and “dextran 5000 ladders” (29 B) and dye 15; “1000” and “5000” correspond to the average molecular masses of dextran oligomers. The time difference between peaks is ca. 1 min. In the case of APTS, the time difference between peaks is ca. 2.3 min (see FIG. 25 “- - -” curve); addition of glucose units' results in roughly the same increase in migration time as for maltose units). The smaller time difference between the peaks is advantageous (more supporting points for a linear alignment curve fit).

FIGS. 30A and B displays electropherograms of the conjugates (reductive amination products) obtained from maltotriose and dyes 6-H and 6-Me before spectral calibration. For both dyes—6-H and 6-Me—the cross-talk between the APTS channel (522 nm) and “595 nm channel” (valid also for 6-H and 6-Me) is quite small; smaller than in the case of dye 15 (FIG. 27). For dye 6-H the cross-talk is ca. 7.8%, and for dye 6-Me—ca. 3.4%. However, even a small-cross talk between the standard and observation channels is prohibitive, as it may cause false positive identifications (of the non-existing analytes).

FIGS. 31A and B shows the electropherograms of the conjugates obtained from “dextran 1000” and “dextran 5000” ladders and dye 6-Me, after spectral calibration. The spectral calibration was based on the use of dyes 6-H and 6-Me conjugated with maltotriose (see FIG. 2, respectively FIG. 4). Their spectral properties and the properties of their conjugates are quite similar. Any cross-talk between APTS color channel (522 nm) the “new” 575 nm channel is absent.

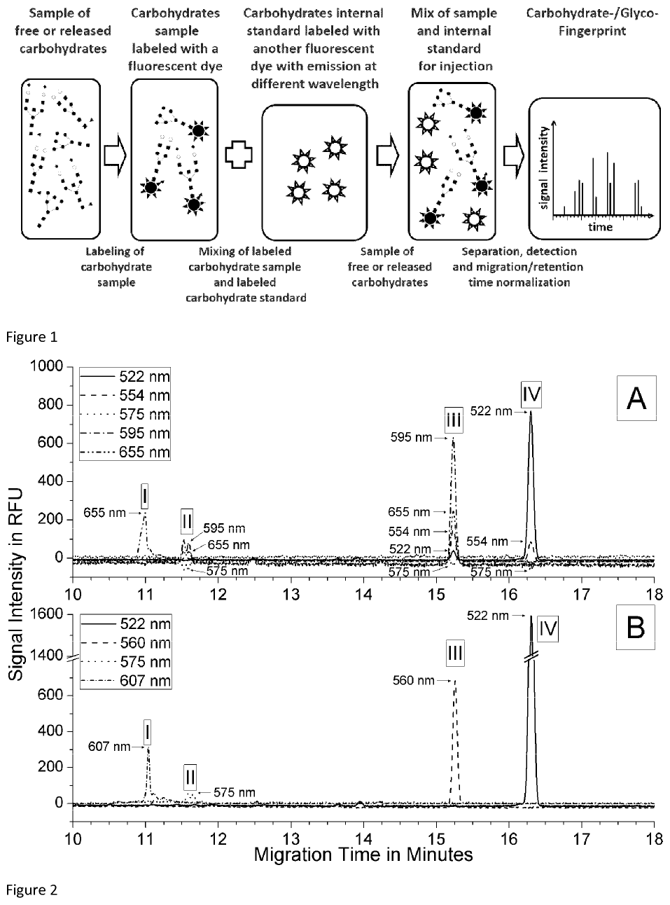

### GENERAL MATERIALS AND METHODS

### Reductive Amination of Carbohydrates

For reductive amination of carbohydrates using the compounds of the present invention, for example the prior art protocol for fluorescent labeling of N-glycans with 8-aminopyrene-1,3,6-trisulfonic acid trisodium salt (APTS) and a reducing agent as published by Hennig R, Rapp E, et al in Methods Molecular Biology in 2015 was used with small adaptations.

The original protocol requires a moderately strong acid (e.g., citric acid as monohydrate; CA) and solvents—dimethyl sulfoxide (DMSO), acetonitrile (ACN) and water (H₂O). Main steps include the preparation of 10-80 mM dye solution in 1.2-3.6 M aqueous CA (solution A) and borane based reducing agent solution in DMSO (solution B). Then it is necessary to mix three components of equal volumes (1-4 μL) of solutions A, B and the sample (free carbohydrates or the carbohydrate moiety of glycoconjugates after release) and incubate at 37° C. for 3-16 h. After completion of the reductive amination, ACN—water mixture (80:20, v/v) is added. For example, if 2 μL of solution A, 2 μL of solution B, and 2 μL of the analyte sample were used, then 50 μL of aq. ACN were added and mixed. This operation provides clear solutions which can be subjected to electrokinetic and/or chromatographic separation-based glycoanalysis.

### Hydrazide Labeling

The hydrazide labeling, using the compounds of the present invention, was performed at 60° C.-80° C. for 1 h-6 h at pH 6-8. A 10-80 mM dye solution was mixed in equal volumes (1-4 μL) with the sample. After completion of the reaction 50 μL of an ACN—water mixture (80:20, v/v) were added. A dilution of the labeling mixture was subjected to electrokinetic and/or chromatographic separation-based glycoanalysis.

### Reactive Carbamate Chemistry

The disuccinimidyl carbonate- or NHS ester-assisted labeling of glycosylamines with compounds of the present invention, was performed at room temperature for 10 60 min at slightly basic pH. Samples were purified by HILIC-SPE as published by Hennig R, Rapp E et al 2015. Purified sample was subjected to electrokinetic and/or chromatographic separation-based glycoanalysis.

### Example 1—Selected Fluorescent Dyes with Large Negative Net Charges and Required Spectral Properties (See Also Scheme 13 and Table 1)

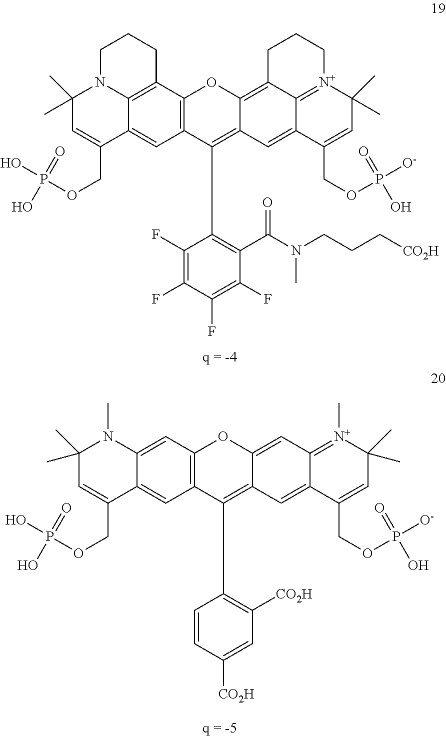 [SMILES: CN(CCCC(=O)O)C(=O)c1c(F)c(F)c(F)c(F)c1C1=c2cc3c4c(c2Oc2c1cc1c5c2CCCN5C(C)(C)C=C1COP(=O)(O)O)CCC[N+]=4C(C)(C)C=C3COP(=O)([O-])O ; CN1c2cc3c(cc2C(COP(=O)(O)O)=CC1(C)C)C(c1ccc(C(=O)O)cc1C(=O)O)=c1cc2c(cc1O3)=[N+](C)C(C)(C)C=C2COP(=O)([O-])O]

The red-emitting rhodamine dye with multiple ionizable groups of structure 20 was obtained by phosphorylation of the corresponding hydroxyl-substituted rhodamine precursor and isolated analogously to compound 19 (another phosphorylated rhodamine dye, see Schemes 6 and 11 above) previously described by K. Kolmakov, et al. in Chem. Eur. J. 2012, 18, 12986-12998 (see compound 7-H therein for the properties and the phosphorylation details). The hydroxyl-substituted precursor for compound 20 was synthesized according to K. Kolmakov, et al. (Chem. Eur. Journal, 2013, 20, 146-157; see compound 14-Et therein). The phosphorylation was followed by saponification of the ethyl ester group via a routine procedure, as described.

Purity and identity of compound 20 was confirmed by the following analytical data: ¹H NMR (400 MHz, DMSO-d₆): δ=1.23 (s, 6H, CH₃), 1.28 (s, 6H, CH₃), 2.62 (s, 6H, NCH₃), 4.21 (m, 4H, 2CH₂), 5.70 (s, 2H), 6.76 (s, 2H), 7.16-7.30 (br. m, 4H), 8.55 (m, 1H), 8.36 (m, 1H) ppm. ¹³C NMR (101 MHz, DMSO-d₆): δ=29.1 (CH₃), 34.2 (CH₃), 95.8 (CH₂), 118.2 (CH), 121.7 (C) 122.6 (C), 125.5 (CH), 127.3 (CH), 127.4 (CH), 128.0 (CH), 129.8 (CH), 133.9 (C), 136, (C), 155.0 (CO), 157.0 (CO) ppm.

¹H NMR (400 MHz, CD₃OD, 20 as a Et₃N-salt): δ=1.12 (t, J=7 Hz, 9H, CH₃CH₂), 1.25 (t, J=7 Hz, 27H, CH₃CH₂), 1.52 (s, 6H, CH₃), 1.53 (s, 6H, CH₃), 3.11, 3.31 (m, 24H, CH₃CH₂), 3.18 (s, 6H, NCH₃), 3.61 (m, 2H, CH₂), 4.45 (m, 2H, CH₂), 6.03 (s, 2H), 6.8 (s, 2H), 6.9 (s, 2H), 7.28 (d, J=8 Hz, 1H), 8.16 (d, J=8 Hz, 1H), 8.66 (m, 1H) ppm. ³¹P NMR (161.9 MHz): δ=−0.2 (DMSO-d₆) and 0.63 (CD₃OD) ppm (s, OP(O)(OH)₂)).

HPLC: t_{R}=3.9 min (Kinetex EVO C-18 column, with 0.02 M aq. Et₃N (A) and 3% MeCN (B), isocratic flow 0.5 mL/min, detection at 254 nm). TLC: R_{f}=0.25 (silica gel plates, MeCN/H₂O 5:1+0.2% Et₃N). HR-MS (ESI): calc. for C₃₅H₃₅N₂O₁₃P₂⁻ ([M-H]⁻) 753.1614, found 753.1672. UV-VIS (PBS buffer, pH=7.4) λₘₐₓ. abs.=582 nm, λₘₐₓ. fl.=609 nm.

### Example 2—Spectral Calibration of Multi-Wavelength Fluorescence Detection Systems to a Set of Four Acridone and Pyrene Based Fluorescent Dyes as Described Herein

For the current example the procedure is exemplarily shown for modified commercial DNA Genetic Analyzer 310, 3100, 3130(xl), 3730(xl) and 3500 (all manufactured by Applied Biosystems, now Thermo Scientific). But, depending on the mode of detection, the here presented re-calibration is also possible for instruments of other manufacturers. The used commercial Genetic Analyzer contains a multiplexed capillary gel electrophoresis (xCGE) unit with laser induced fluorescence detection (LIF), which can (depending on the instrument and operating software) simultaneously detect up to six different fluorescent signals in separate dye channels.

According to the manufacturer virtual filters of the instrument can be calibrated to various pre-defined dye sets like F, D (both: four detection windows) or G5 (five detection windows). As a default spectral calibration for the analysis of oligosaccharides the pre-defined dye set G5 is used [EP 2112506 B1, Ruhaak 2010, Reusch 2015, Feng 2017]. G5 is calibrated to the DS-33 Matrix Standard containing the dyes 6-Fam™ (recorded inside the 522 nm dye trace), VIC® (at 554 nm), NED™ (at 575 nm), PET® (at 595 nm) and LIZ® (at 655 nm). With this calibration APTS labeled oligosaccharides are recorded inside the 6-Fam™ dye trace (522 nm) and the alignment standard GeneScan 500 LIZ™ inside the LIZ® dye trace (655 nm). Unfortunately, using the G5 spectral calibration APTS produces a signal in all other dye traces, as shown in FIG. 2 A for an APTS labeled maltotetraose at 16.3 min. This big cross-talk is caused by the different spectral properties of APTS and 6-Fam™. To be able to perform a migration time alignment without an influencing the cross-talk signal from APTS the GeneScan 500 LIZ™ (LIZ500) is used, as LIZ is recorded inside the dye trace that emits light as far as possible from the APTS channel.

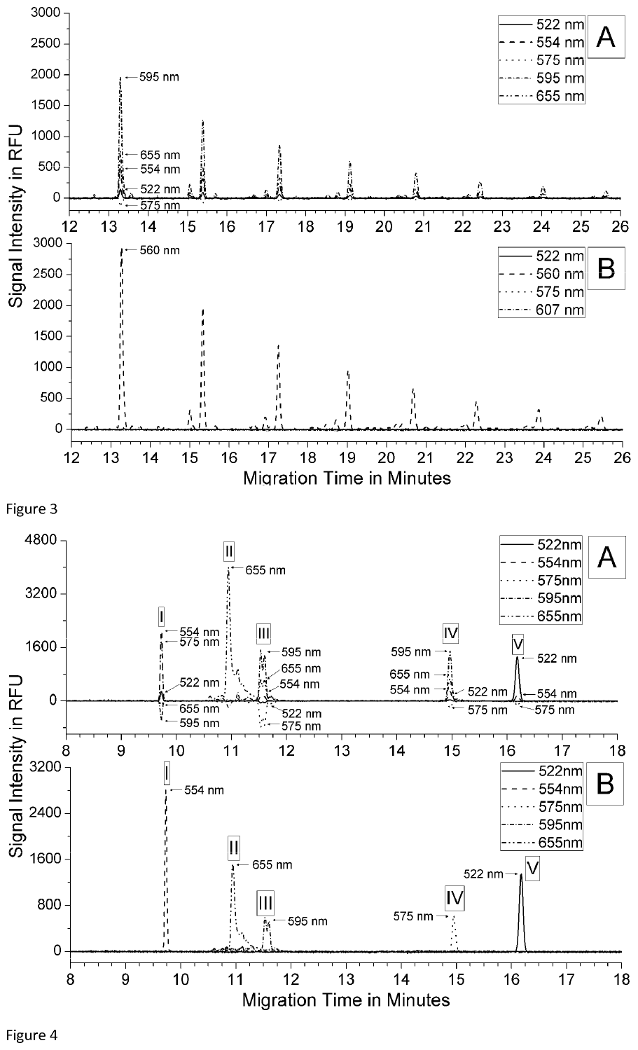

To be able to the use an alignment standard, different from LIZ500 and to reduce the spectral cross-talk the xCGE-LIF instrument was exemplarily calibrated to a set of four dyes, including APTS and three new dyes of the current invention. Before spectral calibration all fluorescent dyes (respectively their oligosaccharide derivates) showed a fluorescent signal in multiple dye traces/channels (FIG. 2 A). Especially, 6-H-labeled carbohydrates showed a big spectral cross talk with all dye channels, as shown for the maltotriose in FIG. 2 A and maltose ladder FIG. 3 A. Consequently, since the use of an internal alignment standard requires the complete absence of fluorescent signal from other dyes inside APTS channel (522 nm), the use of an e.g. 6-H-labeled maltose ladder as an internal alignment standard is not possible without the previous spectral calibration of the instrument. The spectral calibration of the xCGE-LIF instrument to 19, 20, 6-H-labeled maltotriose (6-H^{a}) and APTS-labeled maltotetraose (APTS^{a}) could completely eliminate spectral cross talk (see FIGS. 2 B & 3 B).

After this spectral calibration of xCGE-LIF instrument the 6-H-labeled maltose ladder could be used for internal alignment of APTS labeled carbohydrates. Therefore the 6-H labeled maltose ladder was co-injected with APTS labeled carbohydrates, sensing the same sample background as the APTS labeled carbohydrates. As a side effect, the better fitting spectral calibration results in an increased signal intensity for 6-H labeled ladder (FIG. 3). The signal intensity of the 6-H-maltose peak at 13.2 min increases by a factor of 1.5 (from about 2000 RFU to about 3000 RFU). The same effect could be observed for APTS^{a} in FIG. 2 peak IV at 16.3 min.

A spectral calibration of multi-wavelength systems to a set of four fluorescent dyes is possible to big variation of herein invented dyes, as shown in Table 2.

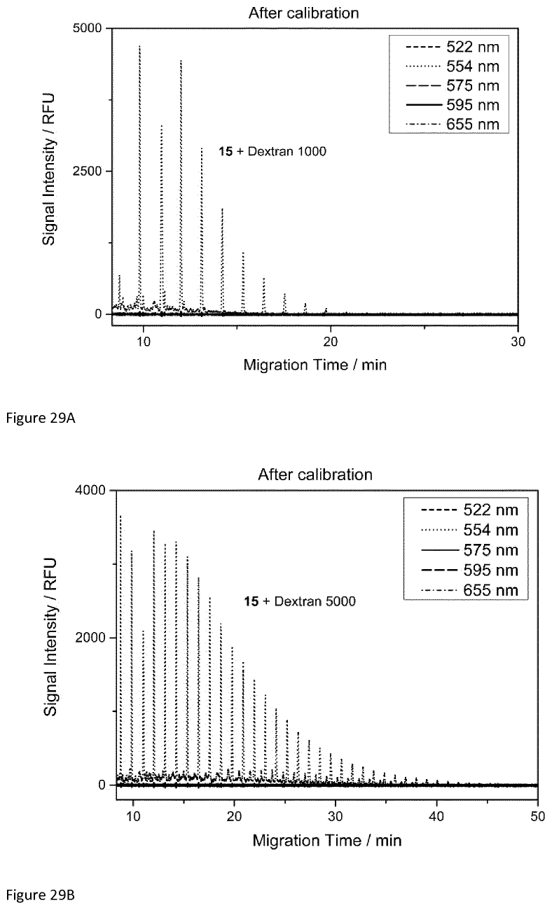

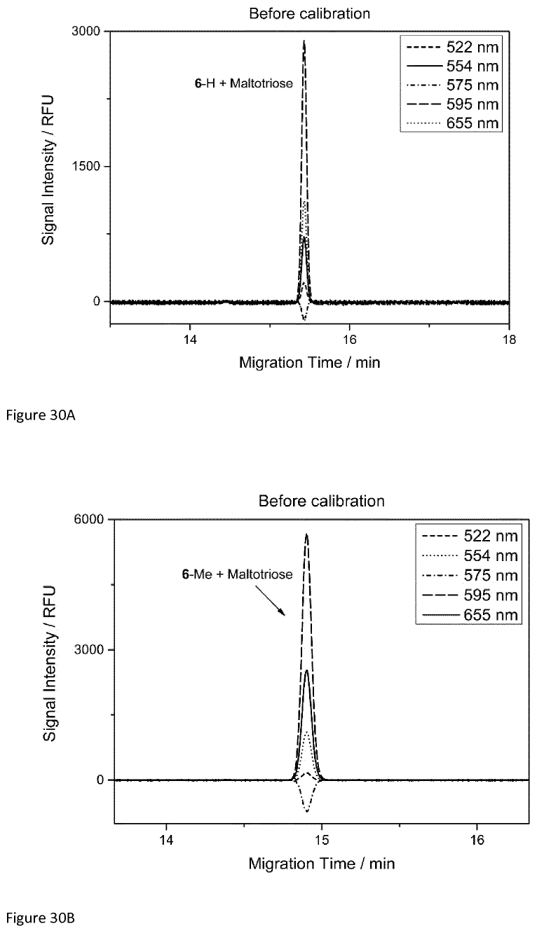

TABLE 2
Spectral calibration of multi-wavelength systems to a set of four dyes.
Exemplarily the possibilities are shown for a four dye spectral calibration of a 3100, 3130, 3130xL,
3730, 3730xL, 3500 and 3500xL instrument. For a spectral calibration one fluorescence dye per trace
needs to be taken, without doubling. E.g. to analyze APTS-labeled samples the spectral trace 522 nm
is calibrated to an APTS-labeled carbohydrate (APTSz). Simultaneous the spectral trace 560 nm is
calibrated to one of the following dye: 6-H, 6-Me, 6-H^{z}, 6-Me^{z}, 8-H, 8-H^{z}, 15, 15^{z}, 23, 23^{z}; the spectral
trace 575 nm to 20, 6-H, 6-Me, 6-H^{z} or 6-Me^{z}, the spectral trace 607 nm to 19 or 20. One possible
spectral calibration is APTS^{z},15^{z}, 6-Me^{z} and 19. These spectral calibration enables the analysis of up
to three samples (APTS-, 15-, and 6-Me-labeled in spectral trace 522 nm, 560 nm and 575 nm)
together with a base pair based internal alignment standard (in spectral trace 607 nm).

| Spectral | | | | | | | | | | |
|---|---|---|---|---|---|---|---|---|---|---|
| trace | Possible fluorescence dye for calibration of spectral trace | | | | | | | | | |
| 522 nm | APTS | APTS^{z} | 15 | 15^{z} | 23 | 23^{z} | | | | |
| 560 nm | 6-H | 6-Me | 6-H^{z} | 6-Me^{z} | 8-H | 8-H^{z} | 15 | 15^{z} | 23 | 23^{z} |
| 575 nm | 6-H | 6-Me | 6-H^{z} | 6-Me^{z} | 20 | | | | | |
| 607 nm | 19 | 20 | | | | | | | | |
| | Small selection of possible combinations for spectral calibration | | | | | | | | | |
| | No. 1 | No. 2 | No. 3 | No. 4 | No. 5 | No. 6 | No. 7 | No. 8 | No. 9 | No. 10 |
| 522 nm | APTS^{z} | APTS^{z} | APTS^{z} | APTS^{z} | APTS^{z} | APTS^{z} | APTS^{z} | APTS^{z} | 23^{z} | 15^{z} |
| 560 nm | 6-H^{z} | 6-Me^{z} | 15^{z} | 15^{z} | 23^{z} | 8-H^{z} | 15^{z} | 23^{z} | 6-Me^{z} | 6-Me^{z} |
| 575 nm | 20 | 20 | 6-Me^{z} | 6-Me^{z} | 6-Me^{z} | 6-Me^{z} | 20 | 6-Me^{z} | 20 | 20 |
| 607 nm | 19 | 19 | 19 | 20 | 19 | 19 | 19 | 19 | 19 | 19 |
| Example | | FIG. 2 | | | | | FIG. 28 | | | |
| for | | and | | | | | | | | |
| spectral | | FIG. 3 | | | | | | | | |
| calibration | | | | | | | | | | |

Index z = fluorescent dye-carbohydrate derivate → 4 e.g. APTS^{z} could be APTS-labeled maltotetraose (see in FIGURE 2), or 15^{z} could be 15-labeled maltotriose (used in FIGURE 4). But ^{z} can be any other carbohydrate, like an O-glycan, N-glycan, milk oligosaccharide, a homopolymer (e.g. maltose, starch, cellulose, dextran) or a heteropolymer (e.g. hemicellulose, arabinoxylan, glucosaminoglycan) build from pentoses and/or hexoses.

### Example 3—Spectral Calibration of Multi-Wavelength Fluorescence Detection Systems to a Set of Five Acridone and Pyrene Based Fluorescent Dyes as Described Herein

For the current example the procedure is exemplarily shown for modified commercial DNA Genetic Analyzer 310, 3100, 3130(xl), 3730(xl) and 3500 (all manufactured by Applied Biosystems, now Thermo Scientific). But, depending on the mode of detection, the here presented re-calibration is also possible for instruments of other manufacturers. The used commercial Genetic Analyzer contains a multiplexed capillary gel electrophorese (xCGE) unit with laser induced fluorescence detection (LIF), which can (depending on the instrument and operating software) simultaneously detect up to six different fluorescent signal in separate dye channels.

The virtual filters of these instruments can be calibrated to various pre-defined dye sets like E5, G5 or D. Thereby, dye set E5 and G5 define five detection windows for five different fluorescent dyes, whereas dye set D defines four detection windows for four different fluorescent dyes. For the analysis of oligosaccharides the pre-defined dye set G5 is used, calibrated to the DS-33 Matrix Standard containing the dyes 6-Fam™ (recorded inside the 522 nm dye trace), VIC® (at 554 nm), NED™ (at 575 nm), PET® (at 595 nm) and LIZ® (at 655 nm) [EP 2112506 B1, Ruhaak 2010, Reusch 2015, Feng 2017]. Subsequently, light emitted by the APTS-labeled oligosaccharides is recorded inside the dye trace 522 nm (Fam™ dye trace) and light emitted by the alignment standard GeneScan 500 LIZ™ (LIZ500) is recorded inside the dye trace 655 nm. As the instrument is not specifically calibrated to the APTS dye, APTS-labeled oligosaccharides emitting light into several dye traces, as shown in FIG. 4 A peak V at 16.3 min for an APTS-labeled maltotetraose, Since the absence of spectral cross-talk between two dye traces is crucial for a proper analysis, this big crosstalk needed to be reduced. Furthermore, to use an oligosaccharide based alignment standard labeled with here invented fluorescent dyes like 15, 6-H, 6-Me, 8-H, or 23, the spectral calibration needed to be customized to theses dyes.

Exemplarily a spectral calibration of the xCGE-LIF instrument was performed to a set of five dyes, as shown in FIG. 4. Before spectral re-calibration (to APTS and four new dyes of the current invention, respectively their oligosaccharide derivates) a big cross talk in multiple dye traces/channels can be observed for all used fluorescent dyes (FIG. 4 A). Especially, 15-labeled (peak I), as well as 6-Me-labeled carbohydrates (peak IV) showed a big spectral cross-talk in all other dye traces, as shown in FIGS. 4 A, 6 A and 7 A. Since the use of an internal alignment standard requires the complete absence of its fluorescent signals inside the APTS channel (522 nm), a spectral calibration of the instrument is necessary. After spectral calibration to 19, 15-labeled maltotriose (15^{a}), 20, 6-Me-labeled maltotriose (6-Me^{a}) and APTS-labeled maltotetraose (APTS^{a}) spectral cross-talk could be completely abolished, as shown in FIGS. 4 B, 5 B, 6 B and 7 B.

Furthermore, the spectral calibration to the dye derivate 15^{a} and 6-Me^{a}enabled the simultaneous use of two different carbohydrate-based standards for the comparison of the alignment performance as shown in FIG. 8. The cross talk between the traces 522 nm (APTS), 554 nm (15) and 575 nm trace (6-Me) is completely absent.

A spectral calibration of multi-wavelength systems to a set of five fluorescent dyes is possible to big variation of herein invented dyes, as shown in Table 3.

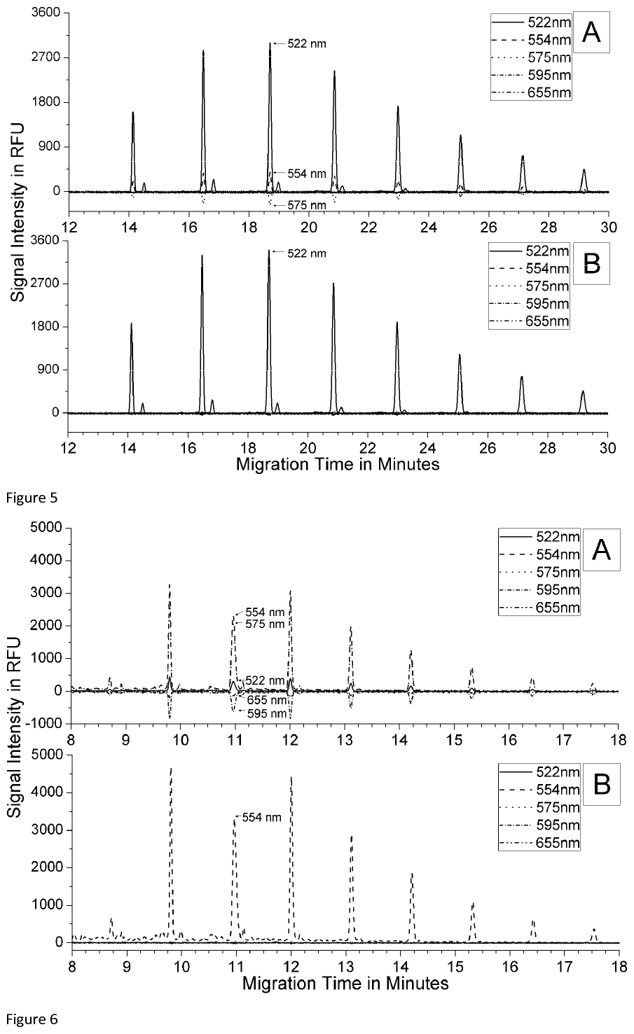

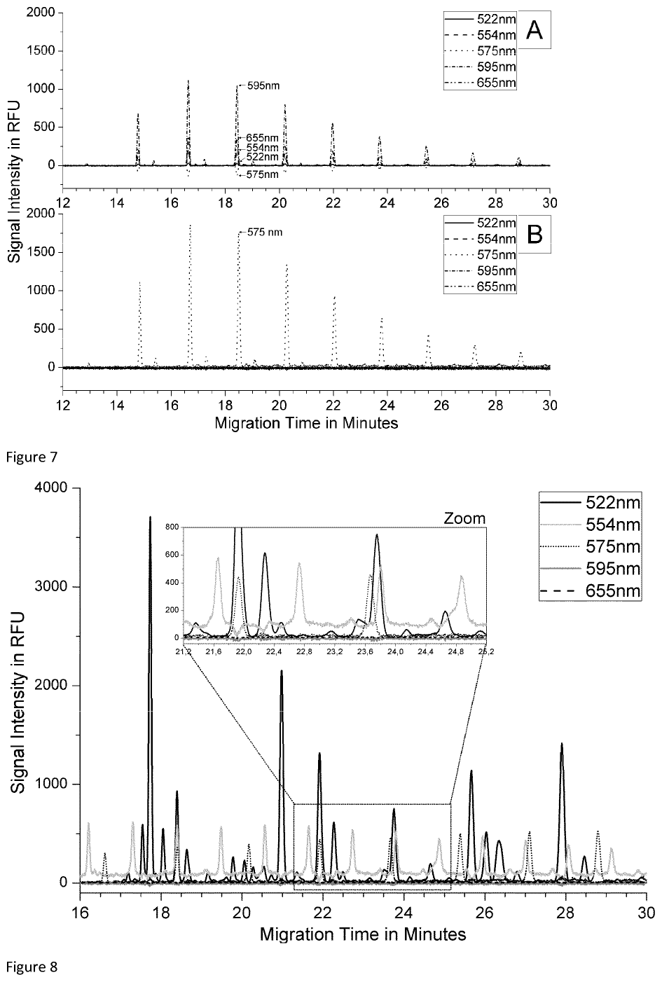

TABLE 3
Spectral calibration of multi-wavelength systems to a set of five dyes.
Exemplarily the possibilities are shown for a five dye spectral calibration of a 3100, 3130, 3130xL,
3730, 3730xL, 3500 and 3500xL instrument. For a spectral calibration one fluorescence dye per trace
needs to be taken, without doubling. E.g. to analyze APTS-labeled samples the spectral trace 522 nm
is calibrated to an APTS-labeled carbohydrate (APTS^{z}). Simultaneous the spectral trace 554 nm is
calibrated to one of the following dye: 8-H, 8-H^{z}, 15, 15^{z}, 23 or 23^{z}; the spectral trace 575 nm to
6-H, 6-Me, 6-H^{z} or 6-Me^{z}, the spectral trace 595 nm to 20 and the spectral trace 655 nm 19. E.g. spectral
calibration to APTS^{z},23^{z}, 6-Me^{z}, 20 and 19 enables the analysis of two samples (APTS-and 23-labeled in
spectral trace 522 nm and 554) together with carbohydrate based alignment standard (6-Me-labeled in
spectral trace 575 nm) and/or a base pair based internal alignment standard (in spectral trace 655 nm).

| Spectral | | | | | | |
|---|---|---|---|---|---|---|
| trace | Possible fluorescence dye for calibration of spectral trace | | | | | |
| 522 nm | APTS | APTS^{z} | | | | |
| 554 nm | 8-H | 8-H^{z} | 15 | 15^{z} | 23 | 23^{z} |
| 575 nm | 6-H | 6-Me | 6-H^{z} | 6-Me^{z} | | |
| 595 nm | 20 | | | | | |
| 655 nm | 19 | | | | | |
| | Selection of possible combinations for spectral calibration | | | | | |
| | No. 1 | No. 2 | No. 3 | No. 4 | | |
| 522 nm | APTS^{z} | APTS^{z} | APTS^{z} | APTS^{z} | | |
| 554 nm | 8-H^{z} | 8-H^{z} | 23^{z} | 15^{z} | | |
| 575 nm | 6-H^{z} | 6-Me^{z} | 6-Me^{z} | 6-Me^{z} | | |
| 595 nm | 20 | 20 | 20 | 20 | | |
| 655 nm | 19 | 19 | 19 | 19 | | |
| Example | | | FIG 15-20 | FIG 4-8, FIG. 15, | | |
| for spectral | | | | 28, 29 and 31 | | |
| calibration | | | | | | |

Index z = fluorescent dye-carbohydrate derivate → 4 e.g. APTS^{z} could be APTS-labeled maltotetraose (see in FIGURE 2), or 15^{z} could be 15-labeled maltotriose (used in FIGURE 4). But ^{z} can be any other carbohydrate, like an O-glycan, N-glycan, milk oligosaccharide, a homopolymer (e.g. maltose, starch, cellulose, dextran) or a heteropolymer (e.g. hemicellulose, arabinoxylan, glucosaminoglycan) build from pentoses and/or hexoses.

### Example 4—Utilizing Acridone Fluorescent Dye Derivates According to the Present Invention for the Internal Migration Time Alignment

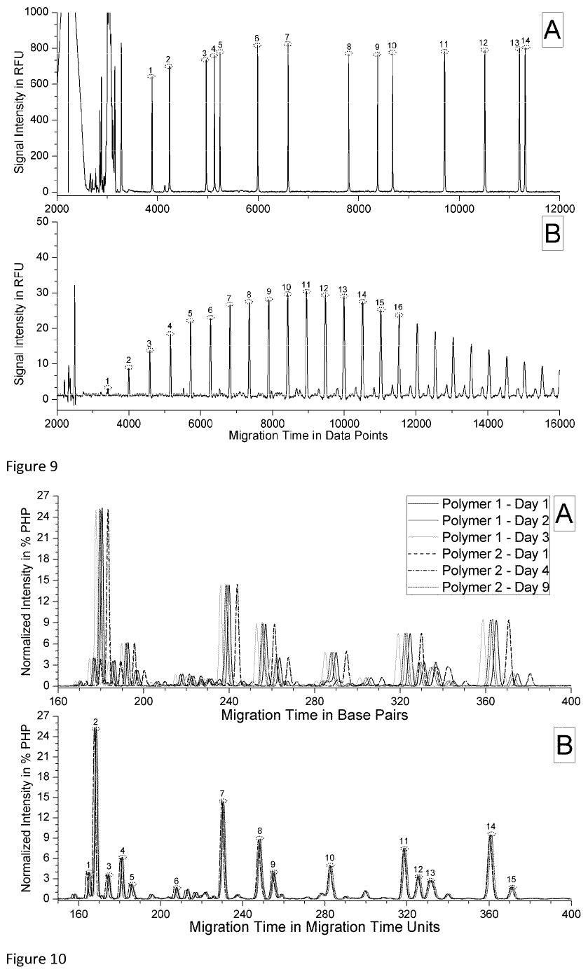

The current example includes the use of modified commercial DNA Genetic Analyzer 310, 3100, 3130(xl), 3730(xl) and 3500 (all manufactured by Applied Biosystems, now Thermo Scientific). Nevertheless, the here presented carbohydrate-based alignment standards can also be used in combination with (single or multiple capillary) CE/CGE instruments or with (U)HPLC instruments of other manufacturers. In general, the migration time alignment of DNA fragment sizes (as used in genomics for e.g. short tandem repeat (STR) or restriction fragment length polymorphism (RFLP) analysis), as well as of carbohydrates in CE/CGE and xCGE is currently realized by the use of base pair size standards, as exemplarily shown in FIG. 9 A (EP 2112506 A1). For this purpose, the migration times of an unknown sample are aligned to a co-injected base pair size standard. For oligonucleotides (DNA/RNA) this internal migration time alignment to a co-injected base pair standard is characterized by a high reproducibility, because the sample background influences the migration times of unknown sample and standard in the same way. Sample and standard are marked with different fluorescent dyes, enabling a wavelength resolved simultaneous detection of both.

While the long-term alignment quality of an unknown DNA fragment to a DNA-based base pair size standard is very good, the long-term alignment quality of oligosaccharides to a base pair size standard is not as good. The aligned migration times of carbohydrates to a base pair size standard show some fluctuation over a longer time and for different polymer lots (see FIG. 10 A). To improve the alignment quality an additional (second) orthogonal alignment step was introduced, using adding bracketing carbohydrate standard(s) (US 2009/028895 A1), as shown in FIG. 10 B.

However, the second (orthogonal) alignment step compensates the most part of these fluctuations in the long-term also for carbohydrates, but not completely. The reason for a less good alignment power in long-term are the different physicochemical properties of the base pair standard and the labeled carbohydrates. While for instance a 360 base pair long fragment (peak 10 in FIG. 9 A) contains 360 nucleotides (deoxyribose+phosphate+nitrogenous base) with 360 negative charges, a fluorescent labeled carbohydrate peak with a similar migration time (peak at 360 base pairs FIG. 10 A) contains only 10 (mono)saccharides with about three negative charges. Consequently, a relatively low charged small molecule is aligned to a highly charged large molecule. Because of their similar mass to charge ratio an alignment is possible. But changing measurement conditions will influence both molecules differently. As a result, the migration times of carbohydrates are variable in long-term after base pair alignment, as shown in FIG. 10 A.

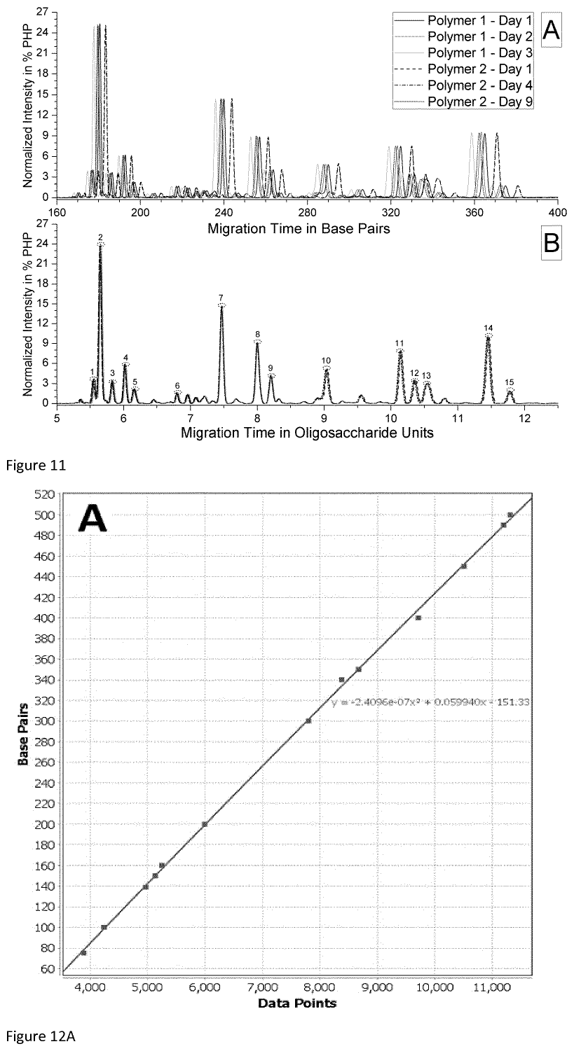

The here presented invention enables the use of a carbohydrate-based standard-mix for the migration time alignment of a carbohydrate. A complete set of new fluorescent dyes was developed to label the oligosaccharide sample and/or these carbohydrate standards/-mix. The new developed fluorescent dyes have different spectral properties than the fluorescent dye used for the labeling of the unknown sample. This enables a co-injection of the fluorescently labeled sample together with the fluorescently labeled carbohydrate alignment standard and a simultaneous detection of both analytes in different dye/wavelength traces as shown in FIG. 8. Compared to the base pair size standard the new carbohydrate-based standards comprise physicochemical properties close/identical to those of the sample. Beside a similar mass to charge ratio, the carbohydrate-based size standards have a similar absolute charge and mass compared to the carbohydrate(s) of the sample. This tremendously improves the long-term reproducibility of the migration time alignment, as shown in FIG. 11 A compared to FIG. 11 B.

For the here presented example human citrate plasma N-glycans were analyzed by xCGE-LIF as described in Hennig et al. 2016 using the dyes as described herein. Briefly, citrate plasma proteins were denaturized and linearized. N-glycans were enzymatically released by PNGase F and labeled with 8-aminopyrene-1,3,6-trisulfonic acid (APTS). After HILIC-SPE purification APTS-labeled N-glycans were analyzed by multiplexed capillary gel electrophoresis with laser-induced fluorescent detection (xCGE-LIF) using an Applied Biosystems® 3130 Genetic Analyzer. For internal migration time alignment APTS-labeled samples were co-injected with a 6-Me-labeled carbohydrate-based alignment standard (6-Me^{b}), see FIG. 11 A or with GeneScan™ 500 LIZ™ dye size standard (LIZ500), see FIG. 11 B.

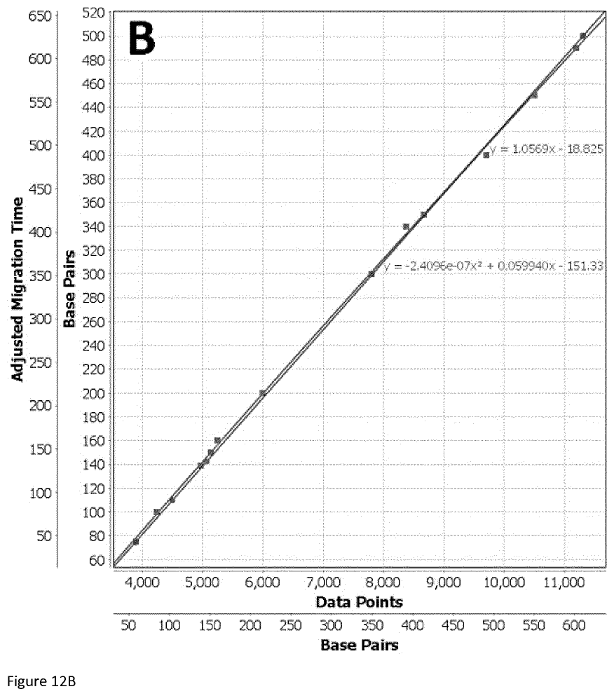

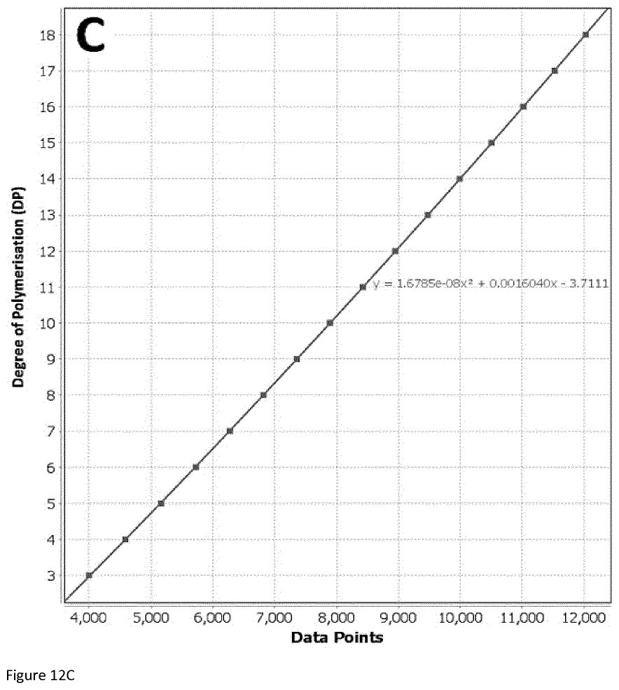

A spectral calibration of the instrument to 15^{a}, 19, 20, 6-Me^{a} and APTS^{a} was performed as described in Example 3. APTS samples were recorded at 522 nm, 6-Me^{b} at the 575 nm and LIZ500 at the 655 nm dye trace. For migration time alignment to LIZ500 13 standard peaks were picked as shown in FIG. 9 A. A 2^{nd} order calibration cure was used for the migration time alignment as shown in FIG. 12 A (EP 2112506 A1). For improved migration time alignment (US 2009/028895 A1) four additional spiked-in bracketing carbohydrate standard peaks were picked and 2^{nd} order calibration curve was adjusted as shown in FIG. 12 B. For migration time alignment to 6-Me^{b} only, 16 standard peaks were picked as shown in FIG. 9 B. A 2^{nd} order calibration cure was calculated as shown in FIG. 12 C and used of the alignment.

By performing an orthogonal adjustment of the LIZ500 alignment as described in U.S. Pat. No. 8,293,084 an improved migration time alignment could be archived (see FIG. 12 B). This improvement could be further enhanced by the use of a carbohydrate-based size standard 6-Me^{b} only as shown in FIG. 12 C. Its superior long-term reproducibility is shown in FIG. 11. While citrate plasma N-glycans aligned to LIZ500 show different migration times depending on the polymer lot and measurement day, the alignment to 6-Me^{b} only shows an almost perfect overlay. To evaluate this in more detail, the 15 biggest peaks of the aligned electropherogram were picked (as shown in FIGS. 10 B and 11 B) and their root-mean-squared error (RMSE) was calculated as shown in Table 4. While the orthogonal second alignment (orthogonal double alignment) could reduce the RMSE by a factor of 4 (3.151% to 0.727%.), an alignment to 6-Me^{b} only could reduce the RMSE by a factor of almost 10 (3.151% to 0.359%). This means using 6-Me^{b} only for the migration time alignment yielded in a 10-fold reduction of the variation, respectively in a 10-fold increase of precision. The smallest RMSE could be archived for single charged N-glycans with 0.236%. But also double charged and neutral N-glycans showed with 0.391%, respectively 0.357% a RMSD really close to this of single charged N-glycans. Thus, acridone dye labeled carbohydrate(only)-based alignment standards like 6-Me^{b} yield the best reproducibility for neutral and low charged oligosaccharides as they can be found on e.g. human proteins like IgG or on recombinant produced monoclonal antibodies (mAb) [Reusch 2015], but they also work for higher charged oligosaccharides. With this high precision and robustness of migration times, independent from polymer age and lot, the method according to the present invention is significantly improved, broader applicable and the built-up and use of a respective database for peak annotation by migration time matching is possible, without the additional orthogonal alignment step as described in Patent US 2009/028895 A1.

TABLE 4
Comparison of alignment precision for N-glycans aligned to a base pair ladder LIZ500, to a
LIZ500 base pair ladder improved by an additional bracketing carbohydrate re-alignment and to an
acridone dye-labeled carbohydrate standard (6-Me^{b}) only. Root-mean-squared-error (RMSD) of citrate
plasma N-glycans was calculated for samples shown in FIG. 10. The 15 picked peaks are depicted
in FIG. 10 B. N-glycan groups contain peaks: 10-15 for neutral, 9-7 for single charged, 2-6 for
double charged and peak 1 for triple charged (for a detailed annotation of glycan peaks see Hennig et
al. 2016). The absolute RMSD is given in base pairs for LIZ500 alignment, in migration time units for
LIZ500 + bracketing carbohydrate (oligosaccharide) re-alignment and in carbohydrate (oligosaccha-
ride) units for 6-Me^{b} only alignment.

| | | | Alignment to | |
|---|---|---|---|---|
| | | | LIZ500 + bracketing | |
| | | Alignment to | carbohydrate | |
| | | LIZ500 as | re-alignment | Alignment |
| | | described in EP | according to US | to 6-Me^{b} |
| | N-glycan group | 2112506 A1 | 2009/028895 A1 | only |
| root-mean- | 15 picked peaks | 8.388 | 1.782 | 0.029 |
| squared error | Neutral N-glycans | 11.226 | 2.168 | 0.037 |
| | Single charged N-glycans | 8.028 | 1.606 | 0.019 |
| | Double charged N-glycans | 5.881 | 1.433 | 0.024 |
| | Triple charged N-glycans | 4.978 | 1.745 | 0.032 |
| root-mean- | 15 picked peaks | 3.151 | 0.727 | 0.359 |
| squared | Neutral N-glycans | 3.326 | 0.660 | 0.357 |
| error in % (of | Single charged N-glycans | 3.158 | 0.658 | 0.236 |
| mean) | Double charged N-gly cans | 3.008 | 0.782 | 0.391 |
| | Triple charged N-glycans | 2.801 | 1.059 | 0.570 |

### Example 5—Utilizing Pyrene Fluorescent Dye Derivates According to the Present Invention for the Internal Migration Time Alignment

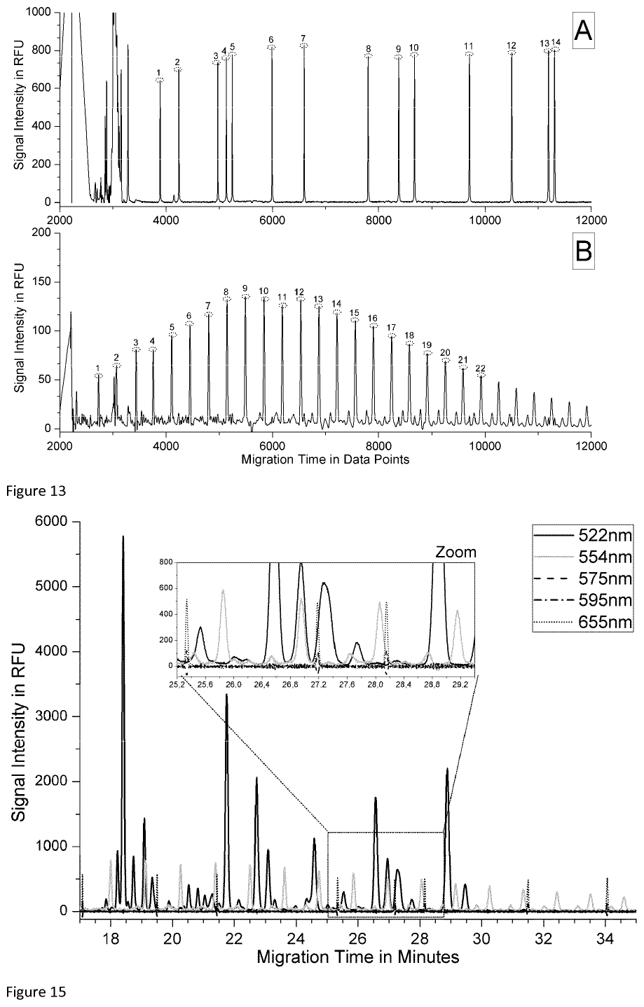

The migration time alignment of DNA fragment sizes as well as of carbohydrates in CE/CGE and xCGE is currently realized by the use of base pair size standards (EP 2112506 A1), as exemplarily shown in FIG. 13 A. For this purpose, the migration times of an unknown sample are aligned to a co-injected base pair size standard. For oligonucleotides (DNA/RNA) this migration time alignment to a co-injected base pair standard is characterized by a high reproducibility, because the migration times of sample and standard are influenced in same way by the same sample background. Sample and standard are marked with different fluorescent dyes, enabling a wavelength resolved simultaneous detection of both.

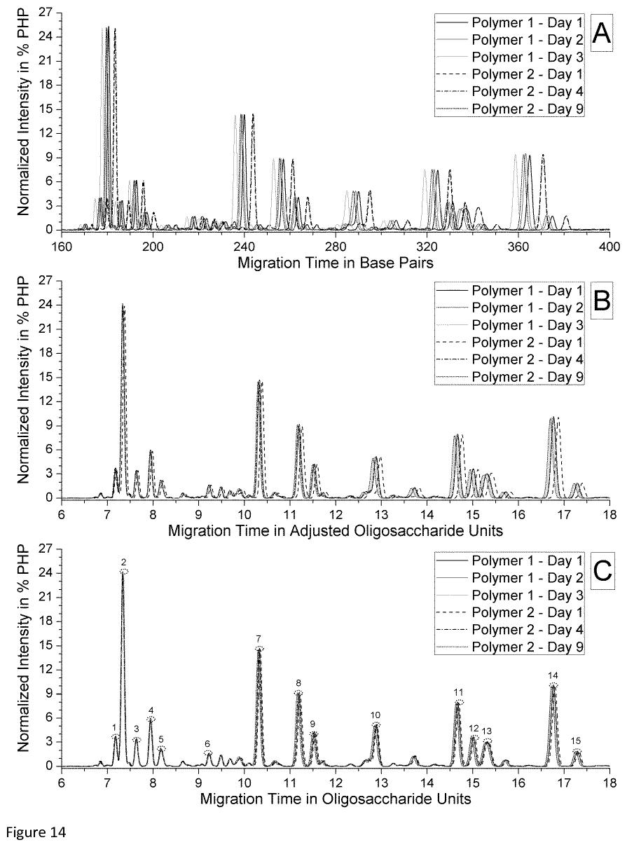

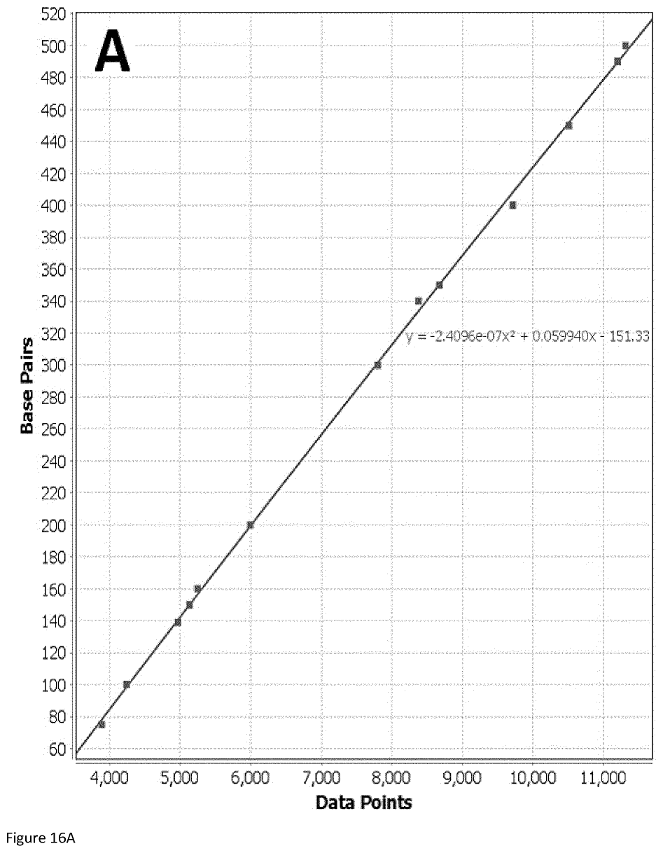

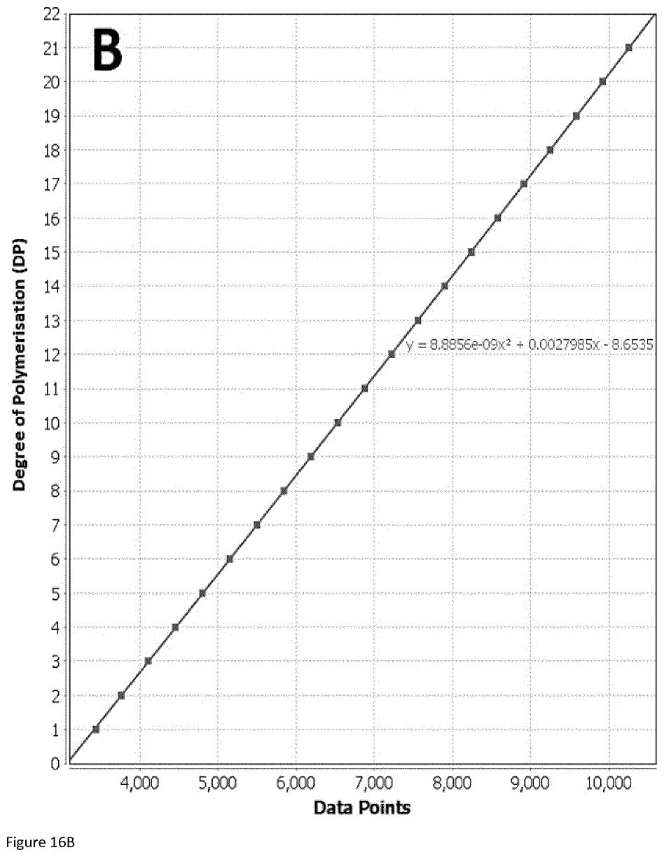

While the long-term alignment quality of an unknown DNA fragment to a DNA based base pair size standard is very good, the long-term alignment quality of carbohydrates to base pair size standards is not as good. The aligned migration times of oligosaccharides to a base pair size standard show some variation over several days and different polymers lots (see FIG. 14 A). To improve the alignment quality, carbohydrate-based alignment standards are needed. Therefore, a complete set of new fluorescent dyes for the labeling of carbohydrates was developed. These newly developed fluorescent dyes comprise spectral properties different from APTS (used for the labeling of sample) and the LIZ, respectively ROX labeled base pair size standard. A spectral calibration of the instrument to 15^{a}, 19, 20, 6-Me^{a} and APTS^{a} (as described in Example 3) allowed a simultaneous detection of the co-injected labeled carbohydrate-sample, the 15-labeled carbohydrate-based alignment standard (15^{b}) and the LIZ 500 base pair standard, as shown in FIG. 15. While APTS labeled samples were recorded at 522 nm, the 15-labeled carbohydrate standard and the LIZ500 base pair standard were recorded simultaneously at the 554 nm, respectively at the 655 nm. Hence both internal standards LIZ500 and 15^{b} could be used for the migration time alignment and directly be compared with each other. For the alignment to LIZ500 13 standard peaks were picked as shown in FIG. 13 A. For migration time alignment to 15^{b} 22 peaks were picked (see FIG. 13 B), covering a similar migration time range as the LIZ500 standard. A 2^{nd} order polynomial fit of picked peaks was performed, as shown in FIG. 16. The considerably improved migration time alignment by using the 15 labeled carbohydrate standard is shown in FIGS. 14 B & C. Compared to base pair-based size standards the new carbohydrate-based size standards comprising physicochemical properties identical to those of the sample. Beside a similar mass to charge ratio, the carbohydrate-based size standards have a similar absolute charge and a similar absolute mass. As a consequence, the use of a carbohydrate-based standard like 15^{b} enables a more precise and reproducible migration time alignment of carbohydrates like N-glycans, O-glycans, glycolipids, human milk oligosaccharides, glycosaminoglycans and other oligosaccharides with a reducing and/or a glycosylamine end.

After alignment to the carbohydrate-based size standard 15^{b} an improved long-term reproducibility could be achieved as shown in FIG. 14 C. While the alignment to the base pair based LIZ500 standard (FIG. 14 A) showed varying migration times for all peaks, depending on the polymer lot and measurement day, the alignment to base pair based LIZ500 standard+15^{b} shows an improved alignment (FIG. 14 B). The best result could be archived by an alignment to 15^{b}, showing an almost perfect overlay (FIG. 14 C). For a more detailed evaluation the 15 biggest peaks were picked inside all samples, as shown in FIG. 14 C. The root-mean-squared error (RMSE) of these 15 peaks in all measurement was calculated as shown in Table 5. Comparing both alignments, the 15^{b} alignment was with a RMSE (in % of mean) of 0.627% five times smaller than the RMSE of 3.151% after LIZ500 alignment. The smallest RMSE could be archived for triple charged N-glycans with 0.236%, indicating that the 15^{b} alignment produces the highest reproducibility for highly charged oligosaccharides as they can be found on e.g. human or recombinant produced erythropoietin (rhEPO) [Meininger 2016], but they also work for lower charged and/or neutral oligosaccharides. Thus, improved precision and robustness of migration times by the 15^{b} alignment, independent from polymer age and lot, allows the built-up and use of an oligosaccharide database for peak annotation by migration time matching, without additional alignment as performed in US 2009/028895 A1. Hence, the method according to the present invention is significantly broader applicable with high precision and robustness of migration times, independent from polymer age.

This improved alignment procedure can also be performed by the use of other oligosaccharide ladders, like chitin, cellulose, maltose, pullulan, glycosaminoglycans, as well as by the use of complex carbohydrates like the glycomoiety of glycolipids, O-glycans, N-glycans and milk oligosaccharides (e.g. lactose, lacto-N-tetraose, lacto-N-hexaose and their fucose and/or lactose elongations).

TABLE 5
Comparison of alignment precision for N-glycans aligned to a base pair ladder LIZ500 (align-
ment to LIZ500), to a base pair ladder improved by an additional carbohydrate re-alignment (alignm. to
LIZ500 + 15^{b}) and to a pyrene dye (15) labeled carbohydrate standard (15^{b}) only. Root-mean-
squared-error (RMSD) of citrate plasma N-glycans was calculated for samples shown in FIG. 12.
The 15 picked peaks are depicted in FIG. 12 C. N-glycan groups contain peaks: 10-15 for neutral,
9-7 for single charged, 2-6 for double charged and peak 1 for triple charged (for a detailed annota-
tion of glycan peaks see Hennig et al. 2016). The absolute RMSD is given in base pairs for LIZ500
alignment, or in carbohydrate (oligosaccharide) units for LIZ500 + 15^{b} and for 15^{b} only alignment.

| | | Alignment to | | |
|---|---|---|---|---|
| | | LIZ500 | | |
| | | As described in | Alignment to | |
| | N-glycan group | EP 2112506 A1 | LIZ500 + 15^{b} | Alignment 15^{b} only |
| root-mean- | 15 picked peaks | 8.388 | 0.121 | 0.078 |
| squared error | Neutral N-glycans | 11.226 | 0.213 | 0.127 |
| | Single charged N- | 8.028 | 0.114 | 0.071 |
| | glycans | | | |
| | Double charged N- | 5.881 | 0.036 | 0.036 |
| | glycans | | | |
| | Triple charged N- | 4.978 | 0.017 | 0.017 |
| | glycans | | | |
| root-mean- | 15 picked peaks | 3.151 | 0.929 | 0.627 |
| squared error | Neutral N-glycans | 3.326 | 1.398 | 0.837 |
| in % (of | Single charged N- | 3.158 | 1.031 | 0.640 |
| mean) | glycans | | | |
| | Double charged N- | 3.008 | 0.442 | 0.445 |
| | glycans | | | |
| | Triple charged N- | 2.801 | 0.241 | 0.236 |
| | glycans | | | |

For the presented example human citrate plasma N-glycans were analyzed by xCGE-LIF as described in Hennig et al. 2016 using the dyes as described herein. Briefly, citrate plasma proteins were denaturized and linearized by incubation with SDS at 60° C. N-glycans were enzymatically released by PNGase F and labeled with 8-aminopyrene-1,3,6-trisulfonic acid (APTS). After HILIC-SPE purification APTS labeled N-glycans were analyzed by multiplexed capillary gel electrophoresis with laser induced fluorescent detection (xCGE-LIF) using an Applied Biosystems® 3130 Genetic Analyzer. A spectral calibration of the instrument to 15^{a}, 19, 20, 6-Me^{a} and APTS^{a} was performed as described in Example 3.

### Example 6—Pyrene and/or Acridone Labeled Carbohydrates as a Universal Alignment Standard

The current example includes the use of modified commercial DNA Genetic Analyzer 310, 3100, 3130(xl), 3730(xl) and 3500 (all manufactured by Applied Biosystems, now Thermo Scientific). Nevertheless, the here presented carbohydrate-based alignment standards can also be used in combination with CE/CGE and with (U)HPLC instruments (single or multiple capillary) of other manufacturers.

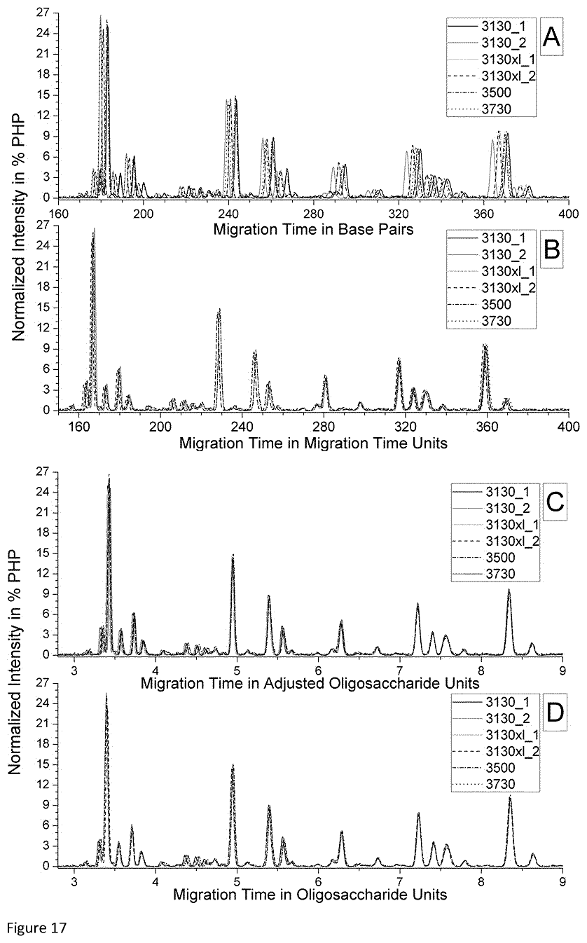

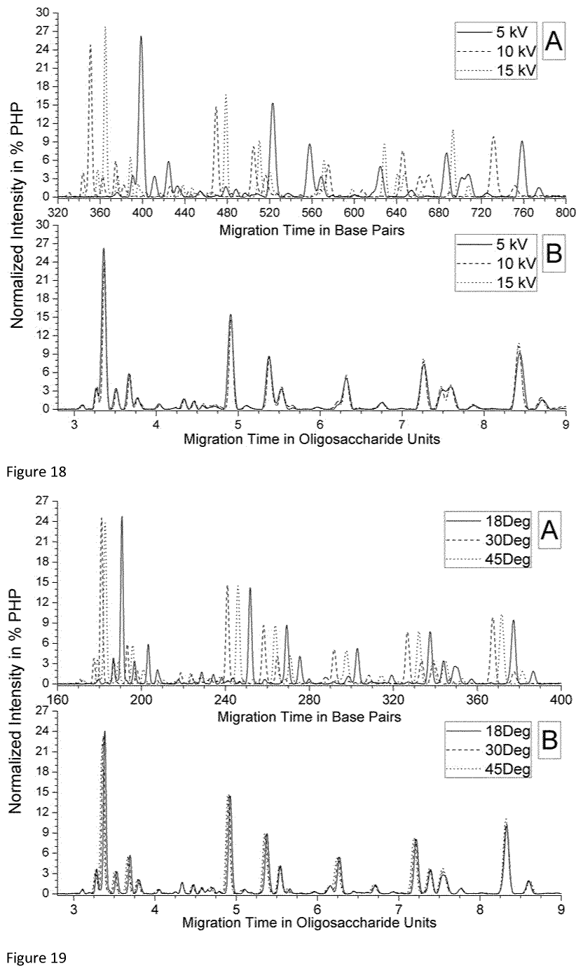

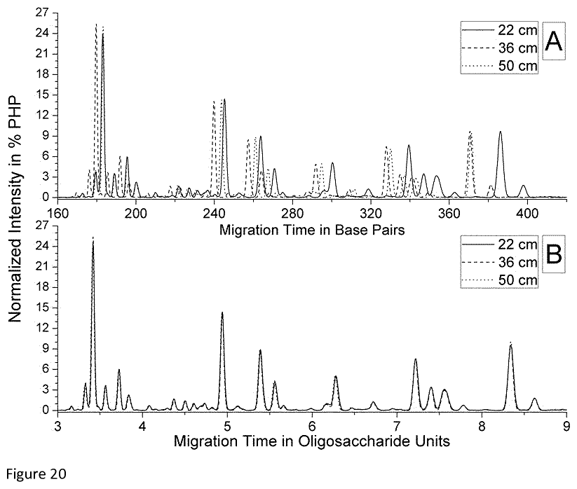

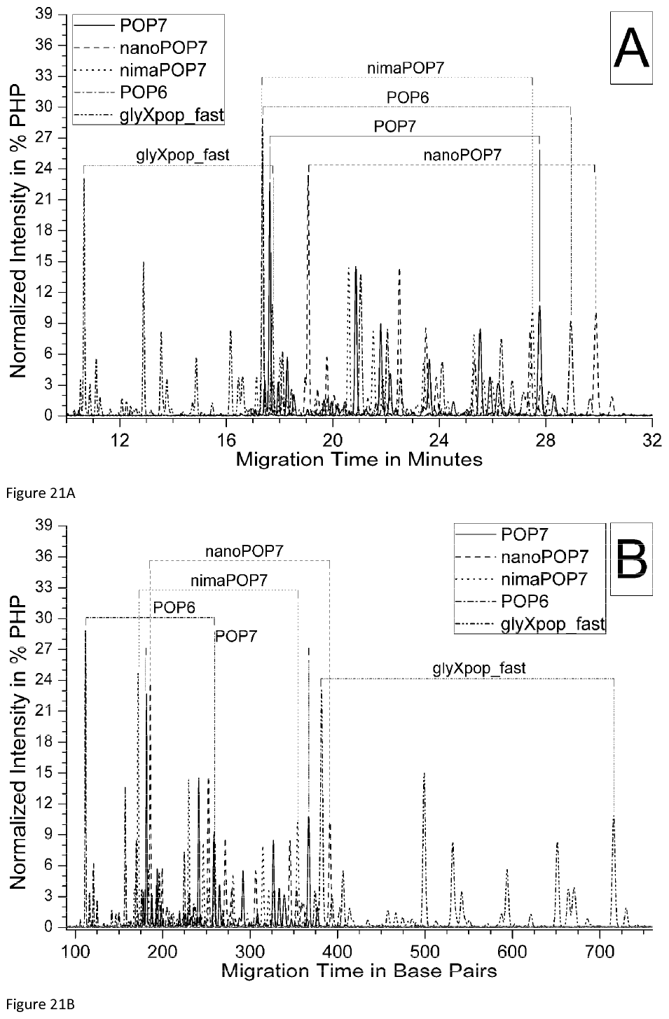

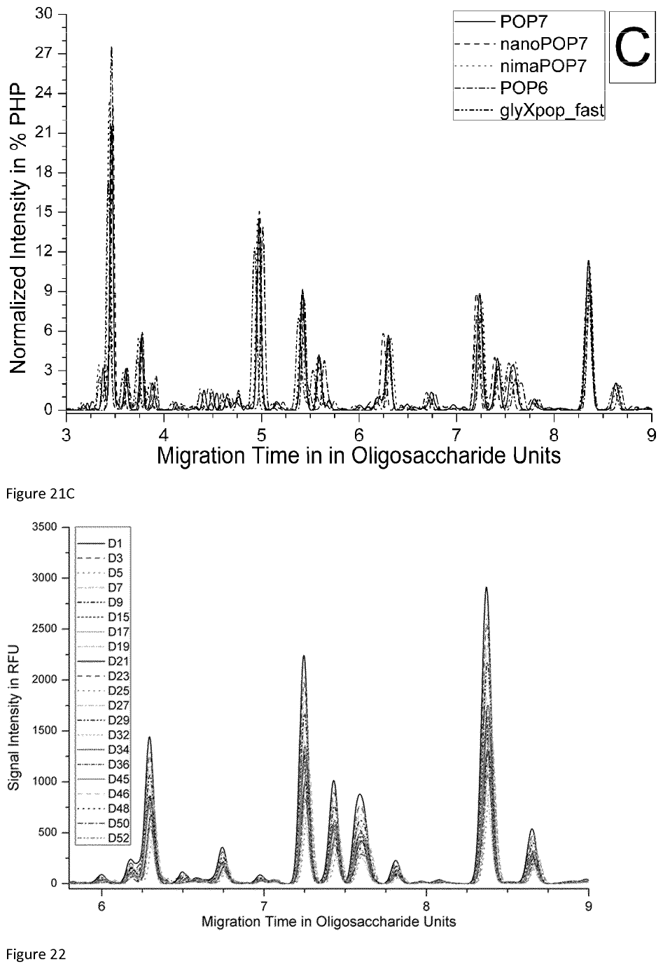

In general, the migration time alignment of DNA fragment and of carbohydrates in (x)CE/(x)CGE is currently realized by the use of base pair size standards (EP 2112506 A1). For this purpose, the migration times of an unknown sample is aligned to a co-injected base pair size standard. While a base pair size standard based alignment shows good results for DNA, the aligned of a carbohydrates sample shows big variations as shown in Example 2 and 3. This variation is more apparent when using different:
Instruments (FIG. 17 and Table 6)
Experimental settings like field strength (FIG. 18) or run temperature (FIG. 19)
Instrument parameters like capillary length (FIG. 20), polymer type (FIG. 21), polymer age (FIG. 22 and Table 6) and polymer lot (Table 6)
   During this stress test these parameters were modified and the alignment procedure (base pairs vs. carbohydrate standard) was compared. For all examples the carbohydrate alignment procedure showed a superior performance. For the most variations a stable migration time could be archived, as shown for example for the different capillary lengths. This means by using the carbohydrate alignment procedure a comprehensive carbohydrate database can be used, also if experimental settings, instrument parameters or instruments are alternated. This is impossible with a base pair-based alignment standard.

TABLE 6
Comparison of alignment precision for N-glycans aligned to a base pair ladder LIZ500
(alignm. to LIZ500), to a LIZ500 base pair ladder improved by an additional bracketing (b) carbohydrate
(oligosaccharide (OS)) re-alignment (alignm. to LIZ500 + bOS, = bracketing OligoSaccharide), to
a LIZ500 base pair ladder improved by an additional pyrene dye (23) labeled carbohydrate standard
(23^{c}) (alignm. to LIZ500 + 23^{c}) and to a pyrene dye (23) labeled carbohydrate standard (23^{c}) only
(alignm. to 23^{c} only). Root-mean-squared-error (RMSD) of citrate plasma N-glycans was calculated for
15 picked peaks as shown in FIGURE 12 C. N-glycan groups contain peaks: 10-15 for neutral, 9-7
for single charged, 2-6 for double charged and peak 1 for triple charged (for a detailed annotation of
glycan peaks see Hennig et al. 2016). The absolute RMSD is given in base pairs for LIZ500 alignment,
in migration time units for LIZ500 + bracketing carbohydrate re-alignment and in carbohydrate units
for LIZ500 + 23^{c} and 23^{c} only alignment. For instrument comparison, data of FIGURE 15 was used (6
different instruments). For polymer lot comparison, citrate plasma N-glycans were measured inside
3130xl1 using four different POP7 polymer lots (lot: 1612560, 1701565, 1703117 and 1705571). For
polymer age comparison citrate plasma N-glycans were measured inside 3130xl_1 with fresh polymer
(lot: 1708574), fresh opened one year old polymer (lot: 1411512), opened one year old polymer
(lot: 1411512) and opened five years old polymer (lot: 1208456). For all comparison cases a
reduction of RMSD by a factor of five (10.697 to 2.172) up to seven (2.246 to 0.334) could be archived.

| | | Instrument Comparison | | | | Polymer Lot | | Polymer Age | |
|---|---|---|---|---|---|---|---|---|---|
| | | (see Figure 17 A, B, C & D) | | | | Comparison | | Comparison | |
| | | | Alignm. | Alignm. | Alignm. | | Alignm. | | Alignm. |
| | | Alignm. | To | To | To | Alignm. | To | Alignm. | to |
| | N-glycan | to | LIZ500 + | LIZ500 + | 23^{c} | To | 23^{c} | To | 23^{c} |
| | group | LIZ500 | bOS | 23^{c} | only | LIZ500 | only | LIZ500 | only |
| root- | 15 peaks | 4.446 | 1.133 | 0.018 | 0.013 | 5.905 | 0.015 | 31.838 | 0.100 |
| mean- | Neutral | 5.365 | 1.060 | 0.010 | 0.007 | 7.722 | 0.010 | 45.485 | 0.053 |
| squared | Single | 4.240 | 1.225 | 0.015 | 0.017 | 5.687 | 0.013 | 29.895 | 0.109 |
| error | charged | | | | | | | | |
| | Double | 3.646 | 1.125 | 0.027 | 0.017 | 4.283 | 0.020 | 19.606 | 0.144 |
| | charged | | | | | | | | |
| | Triple | 3.547 | 1.334 | 0.035 | 0.024 | 3.764 | 0.027 | 16.942 | 0.129 |
| | charged | | | | | | | | |
| root- | 15 peaks | 1.715 | 0.487 | 0.417 | 0.298 | 2.246 | 0.334 | 10.697 | 2.172 |
| mean- | Neutral | 1.572 | 0.318 | 0.137 | 0.089 | 2.296 | 0.126 | 12.111 | 0.689 |
| squared | Single | 1.665 | 0.505 | 0.284 | 0.325 | 2.251 | 0.240 | 10.785 | 2.036 |
| error in | charged | | | | | | | | |
| % (of | Double | 1.860 | 0.614 | 0.707 | 0.445 | 2.204 | 0.540 | 9.292 | 3.711 |
| mean) | charged | | | | | | | | |
| | Triple | 1.995 | 0.816 | 1.050 | 0.739 | 2.136 | 0.829 | 8.973 | 3.783 |
| | charged | | | | | | | | |

### Example 7—Recalibration of a DNA Sequencer Using New Sets of Fluorescent Acridone and Pyrene Dyes According to the Invention

Commercial CE-systems may have a multi-wavelength detector and therefore several color channels.

There are so-called “virtual light filters” in those systems, where the software defines certain wavelength-areas for the collection of the fluorescent emissions from different dyes.

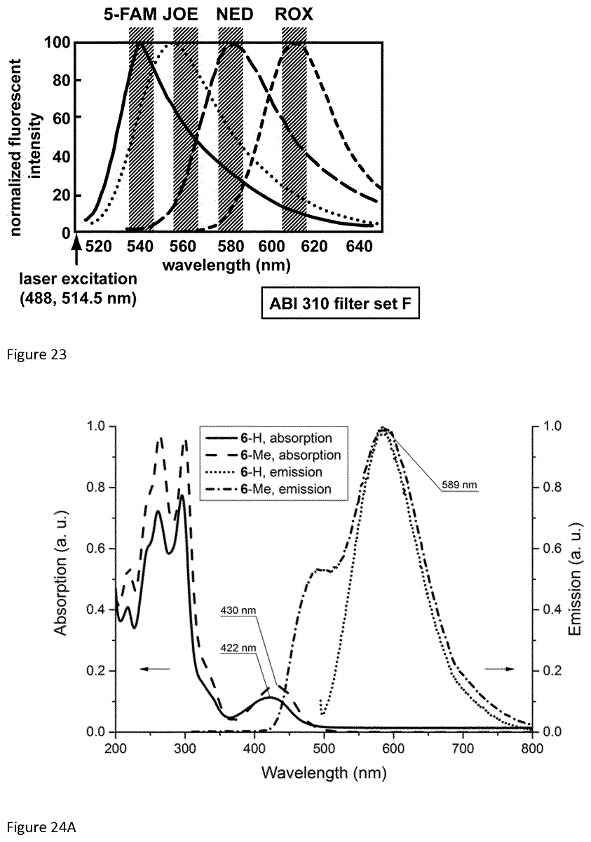

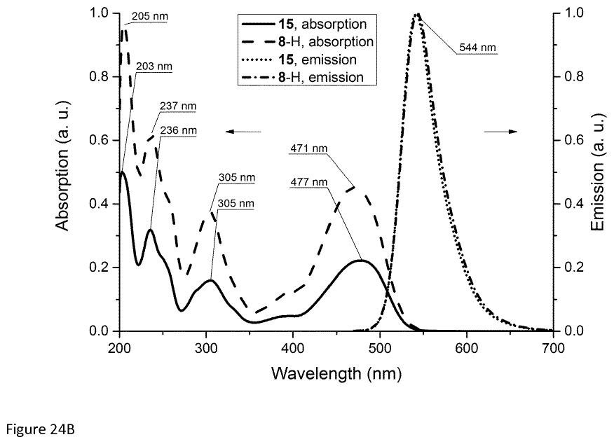

These areas are called virtual filters. Each of them is associated with a relatively narrow range of the visible light emitted only by one dye (FIG. 23). The main data set from the DNA sequencer has 4 color traces (FIG. 23) corresponding to four nucleotides. In fact, there can be any number of virtual filters, since the filter is simply a software-designated site on the CCD array. Since a dye's emission profile is always rather broad, a part of it is registered by virtual filters other than the one intended to collect its emission maximum. The dyes in each set are selected in such a way that they have widely spaced emission maximums, in order to minimize overlap of the emission profiles on the CCD array. However, the spectral overlap still occurs to some extent, and a certain cross-talk is always present. On the other hand, each position of the DNA sequence has only one of four nucleotides, and in the course of sequencing each of them is detected in its “own” color channel. Therefore, the problem of cross-talk is much less important for DNA sequencing than for glycan analysis, because four lanes of the DNA sequencing contain peaks with similar intensities, and only one color trace has a prominent peak at a certain place.

Importantly, the emission of APTS dye and its conjugates with glycans always appears in the channel with shortest wavelength, and the absence of cross-talk with the reference channel is crucial. After labeling with APTS, the electropherograms of the complex glycan mixtures contain peaks with intensities varying in the orders of magnitude. Thus, the fluorescence signal in APTS channel has to be completely free from the emission “leaking” from the reference channel. The reference sample contains a mixture labeled with another fluorescent dye and injected simultaneously with the analyzed sample. This requirement of a “complete” absence of the cross-talk between the observation channel (APTS dye or its substitute) and the reference channel seems to be easy to fulfill, but is not the case, because both dyes have to be excited with the same light source and their emission spectra overlap. Up to now, a LIZ dye (attached to a “DNA ladder” used as an internal alignment standard in glycan analysis) was used as an additional color in a 655 nm observation channel. For the detection of a LIZ dye, a virtual filter set G5 (including 6-Fam™, VIC®, NED™, PET® and LIZ®) is used in ABI 3100 DNA sequencer (ABI user manual). This dye consists of a FRET pair—a donor dye, and an acceptor dye. This combination (similar to a dye with very large Stokes shift) provides an absence of cross-talk, because a donor dye is efficiently excited with green light, transfers energy to an acceptor, and the latter emits only red light. However, FRET pairs with complete energy transfer, multiple negative charges, and an aromatic amino group are too complex and therefore hardly synthetically available. Therefore, the present invention provides fluorescent dyes with enlarged Stokes shifts. As substitutes for an internal alignment standard, these dyes give no emission in the APTS (observation) channel.

In order to eliminate cross-talk with an APTS channel, it was necessary to re-calibrate the commercial DNA sequencer (manufactured by Applied Biosystems) using other sets of fluorescent dyes. According to the manufacturer, there can be any number of (various) virtual filters (observation windows). Therefore, the new detection channels may be designated. For example, the emission maxima of 5 arbitrary fluorescent dyes define 5 (new) detection windows (filters). To minimize cross-talk, the absorption maxima of the new reference dyes have to be spread more or less uniformly in the range from 500 nm to 655 nm. The “crosstalk” (overlap) between emission colors on the CCD array is corrected by a matrix file in the software. This procedure is well-known and called “linear unmixing” (T. Zimmermann, et al., Methods Mol. Biol. 2014, 1075, 129-148).

The matrix file is generated from a separate, “matrix” run in which the reference dyes or their derivatives are subjected to capillary electrophoresis, separated into individual peaks and their emission spectra are registered in the whole spectral range. The matrix file contains information about the inputs of the individual dyes into the emitted light falling onto a certain filter (detected within a certain observation window). For each filter (detection window), the input of one dye is maximal, but there are also contributions from the other dyes “contaminating” the overall signal passing through the certain filter.

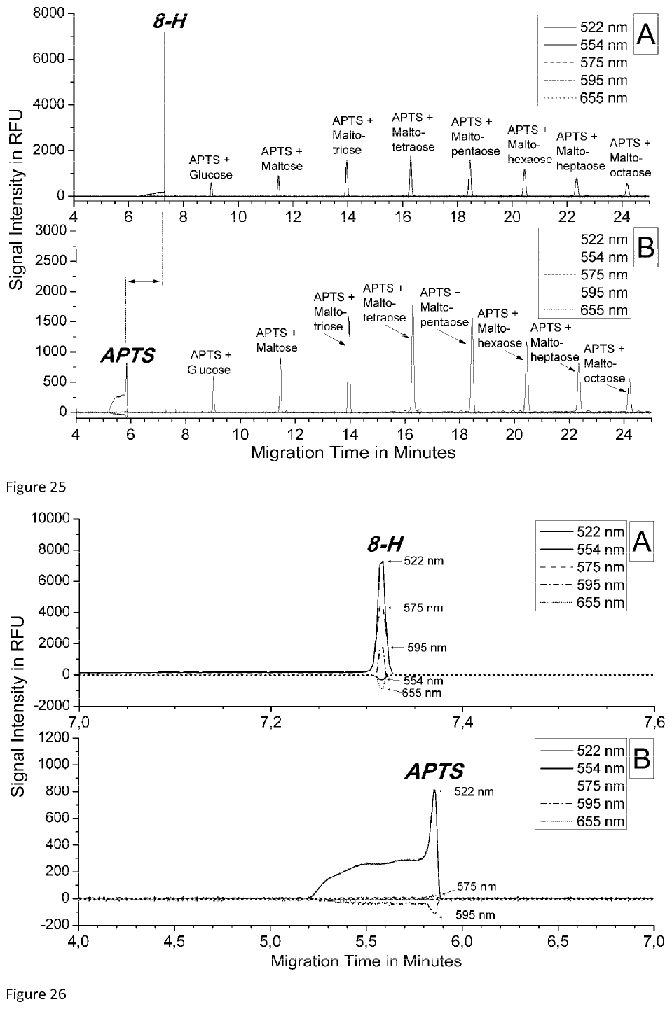

In FIG. 25 a comparison of the dyes 8-H (tri-phosphorylated aminopyrene) and APTS (tri-sulfated aminopyrene) is shown. The spiked-in APTS labeled maltose ladder (to both samples) provides a time orientation. The retention time of 8-H is higher than the retention time of APTS, though the m/z ratio for 8-H (144) is lower than that of APTS (151). In APTS, the charged groups (sulfonic acid residues) are directly attached to fluorophore. The presence of N-methyl-N-(2-hydroxyethyl) linker in 8-H increases the hydrodynamic ratio of the dye, and this explains higher retention time of the free dye 8-H.

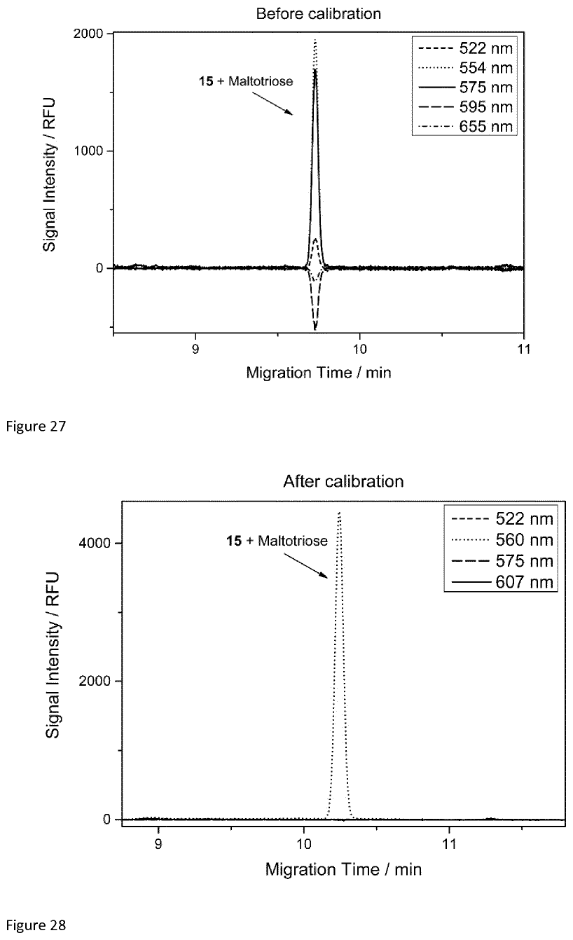

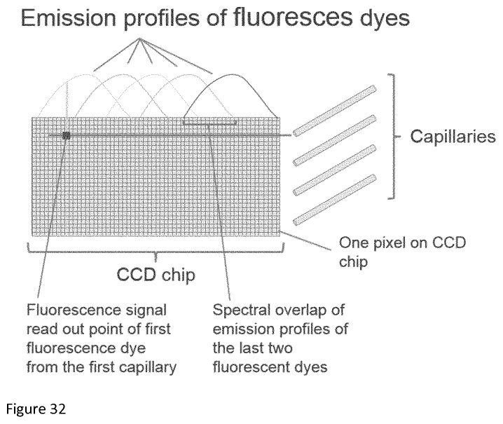

FIG. 26 shows a zoom-in to peaks of 8-H und APTS. This figure was obtained before spectral calibration. Due to the strong cross-talk of 8-H with the APTS color channel (522 nm; black in FIG. 26 A), the dye 8-H cannot be used together with APTS in any analytical assays. The same is true for the tri-phosphorylated pyrene dye 15 as shown in FIG. 27 and the di-phosphorylated acridone dyes 6-Me and 6-H as shown in FIG. 30. Therefore, a new color calibration of the DNA sequencer is necessary, in order to reduce or, if possible, fully eliminate cross-talk between the emission channels attributed to APTS and triphosphorylated pyrene dyes 6-H, 6-Me or 8-H and 15.

For that, the negatively charged fluorescent dyes 19, 20, 6-R and 15 (see below) were chosen and used together with APTS in a new set for the spectral calibration of the electrophoresis unit integrated into a DNA sequencing device. With these dyes, a new matrix file was generated and used in correcting the spectral overlap.

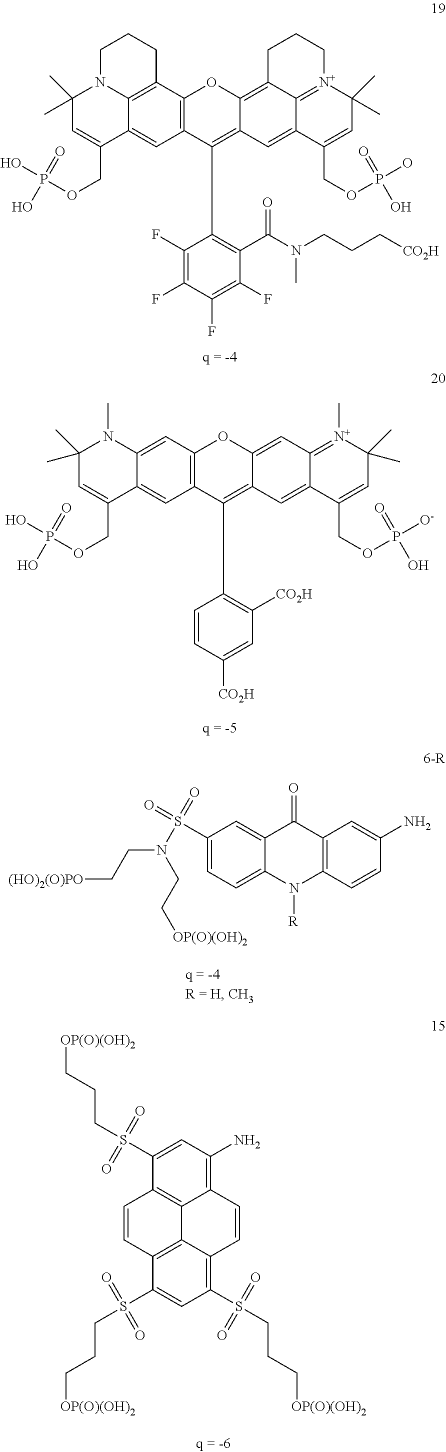

Table 7 indicates the properties of fluorescent dyes, including rhodamines 19 and 20 (see K. Kolmakov, et al., Chem. Eur. J. 2012, 18, 12986-12998 and K. Kolmakov, et al., Chem. Eur. Journal, 2013, 20, 146-157.), 6-R and 15 and their conjugates with oligosaccharides consisting of maltose units. Remarkably, the conjugate of dye 8-H with maltohexaose has a much shorter retention time (13.1 min) that the APTS derivative obtained from maltotetraose (16.5 min). Though the hydrodynamic ratios of dyes 8-H and 15 are larger than that of APTS, the presence of six negative charges in these dyes (versus three in APTS) strongly increases their electrophoretic mobilities in the electric field.

TABLE 7
Properties of fluorescent dyes 6-R, 15, 19, 20 and 23 used in a new set together with APTS
for the spectral calibration of the fluorescence detection unit integrated into a DNA sequencing device.

| | | | | Migration time,^{b} |
|---|---|---|---|---|
| | Free dye absorption | Free dye emission | | (see also FIGS. in |
| Dye | λₘₐₓ, nm (ε, M⁻¹ cm⁻¹) | λₘₐₓ, nm (ϕ_{fl}) | Conjugate with | attachment) |
| 6-H^{a} | 217 (13500), 260 (26000) | 586 (0.05) | maltotriose | 15.5 min, 575 nm |
| | 295 (28000), 420 (3700) | 2 × OP(O)(OH)₂ | | |
| 6-Me^{a} | 219 (10300), 263 (18600) | 585 (0.05) | maltotriose | 15.0 min, 575 nm |
| | 299 (18500), 430 (2900) | 2 × OP(O)(OH)₂ | | |
| 8-H^{a} | 465 (3 × OP(O)(OH)₂) | 530 (0.94) | free dye | 7.3 min, 522/544 nm^{c} |
| | | | maltohexaose | 13.1 min, 554 nm |
| 15^{a} | 477 (3 × OP(O)(OH)₂) | 542 (0.94) | free dye | 6.8 min, 554 nm |
| | | | maltotriose | 9.5 min, 554 nm |
| APTS^{a} | 425 (3 × SO₃H) | 457 | maltotetraose | 16.5 min, 522 nm |
| 19 | 635 (75000) | 655 (0.55)^{b} | free dye | 11.2 min |
| 20 | 581 (60000) | 607 (0.95) | free dye | 11.7 min |
| 23^{a} | 486 (23000) 3 × SO₃H | 542 (0.83) | free dye | 9.9 min, 554 nm |
| | | | maltotriose | 16.9 min, 554nm |

^{a}Conjugation to carbohydrates and/or N-alkylation of amino-substituted dyes shifts the absorption and emission bands to the red spectral region by ca. 20 nm (see Table 1).
^{b}Retention (migration) time in the additional color channel where the dye has the largest emission, as measured in a gel at pH = 8.
^{c}Conjugates of dye 8-H have a large cross-talk between 522 and 544 nm channels.

In fact, if one compares the emission maxima for the color channels in FIG. 24, on one hand, and the color channels in Table 7, one may conclude that these are very similar. Small differences in the emission maxima are present only for “575 nm channel”, and even smaller—for “595 nm channel”. The new emission band which served for the definition of “575 nm channel” (FIG. 27 vs. 28) is very broad. The emission maximum of the “new 595 nm channel” is slightly red-shifted (from 595 nm to ca. 607 nm). However, these small differences enabled to fully eliminate any cross-talk.

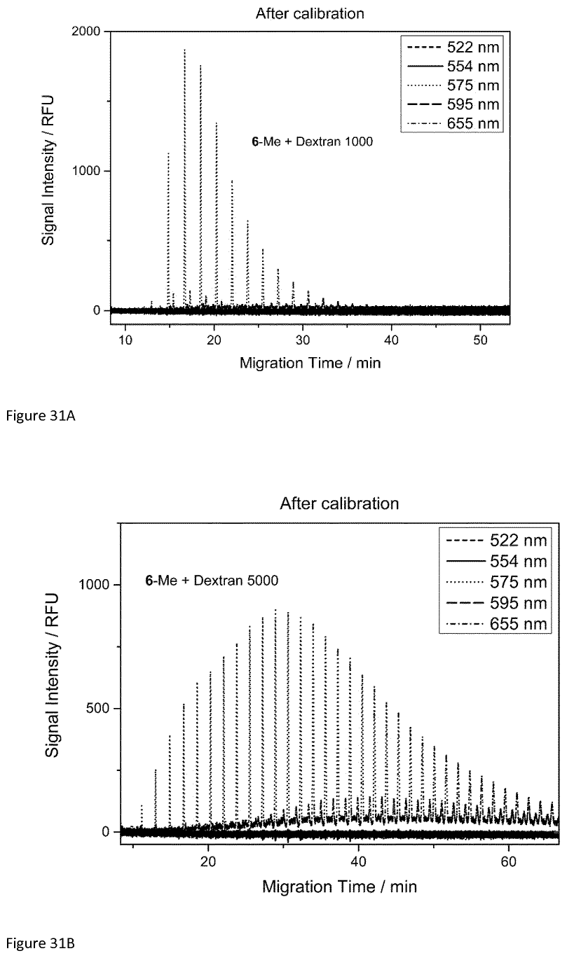

For obtaining the color traces depicted in FIG. 29, five new virtual filters were set in a DNA sequencer (Table 3). The most short wavelength channel corresponds to all APTS conjugates (522 nm), the next one—to the emission maximum of pyrene 15—maltotriose conjugate (554 nm; valid for all conjugates of dye 15), a “green” one—to all conjugates of acridone dyes 6-H and 6-Me with reducing sugars (575 nm), another one corresponds to the emission maximum of the free dye 20 (595 nm, FIG. 4), and, finally, a “red” channel was chosen according to the emission of dye 19 (655 nm; FIG. 4). By this choice, any kind of cross-talk between APTS channel (522 nm) and 554 nm channel, as well as between APTS channel (522 nm) and 575 nm (green) channel was eliminated (see FIGS. 29 and 31)

FIGS. 29 A and B shows the electropherograms of the conjugates obtained from the mixtures of carbohydrates (“dextran 1000” (A) and “dextran 5000 (B) ladders”) and dye 15; “1000” and “5000” correspond to the average molecular masses of dextran oligomers. The time difference between peaks is ca. 1 min. In the case of APTS, the time difference between peaks is ca. 2.3 min (see FIG. 25; addition of glucose units' results in roughly the same increase in migration time as for maltose units). The smaller time difference between the peaks is advantageous, if the fluorescent dye is intended for the generation of the new internal standard mixture.

FIGS. 30 A and B displays electropherograms of the conjugates (reductive amination products) obtained from maltotriose and dyes 6-H (A) and 6-Me (B) before color calibration. For both dyes—6-H and 6-Me—the cross-talk between the APTS channel (522 nm) and “595 nm channel” (valid also for 6-H and 6-Me) is quite small; smaller than in the case of dye 15 (FIG. 27). For dye 6-H the cross-talk is ca. 7.8%, and for dye 6-Me—ca. 3.4%. However, even a small-cross talk between the standard and observation channels is prohibitive, as it may cause false positive identifications (of the non-existing analytes).

FIGS. 31 A and B shows the electropherograms of the conjugates obtained from “dextran 1000” (A) and “dextran 5000” (B) ladders and dye 6-Me, after spectral calibration (see Example 3). The new color calibration was based on the use of dyes 6-H and 6-Me conjugated with maltotriose. Their spectral properties and the properties of their conjugates are quite similar. Any cross-talk between APTS channel (522 nm) and the new “575 nm” channel is absent.

For dye 6-Me (and 6-H), the time difference between peaks is ca. 1.5 min, which corresponds to four negative charges on the dye residue. The right side of FIG. 31 shows peaks with migration times up to 60 min and more; these indicate that dyes 6-Me (and 6-H; the data are similar and therefore not shown) may be favorably compared with APTS (FIG. 25).

### LITERATURE

Feng H T, et al., Electrophoresis (2017) 38, 1788-1799. doi: 10.1002/elps.201600404. Epub 2017 May 11.
Hennig R, et al., Biochimica et Biophysica Acta—General Subjects 2016, 1860, 1728-1738.
Hennig R, et al., Methods Molecular Biology 2015, 1331, 123-143.
Meininger M, et al., Journal of Chromatography B 2016, 1012, 193-203.
Reusch D, rt al., MAbs. 2015, 7, 167-179. doi: 10.4161/19420862.2014.986000.
Ruhaak L R, et al., Journal of Proteome Research 2010, 9, 6655-6664.

1. A method for an automated determination and/or identification of carbohydrates and/or carbohydrate mixture composition pattern profiling comprising the steps of:
a) obtaining a sample containing at least one carbohydrate;
b) labelling said carbohydrate(s) with a first fluorescent label;
c) providing a standard of known composition labelled with a second fluorescent label;
d) determining the migration/retention time(s) of said carbohydrate(s) and the standard of known composition using electrokinetic/chromatographic separation techniques combined with fluorescence or laser induced fluorescence detection;
e) aligning the migration/retention time(s) to migration/retention time indice(s) based on given standard migration/retention time indice(s) of the standard;
f) comparing these migration/retention time indice(s) of the carbohydrate(s) with standard migration/retention time indice(s) from a database;
g) identifying or determining the carbohydrate(s) and/or the carbohydrate mixture composition pattern,
wherein the standard composition is added to the sample containing the unknown carbohydrate and/or carbohydrate mixture composition, the first fluorescent label and the second fluorescent label are different and wherein the first fluorescent label or the second fluorescent label is a fluorescent dye, preferably having multiple ionizable and/or negatively charged groups
which is selected from the group consisting of compounds of the following general Formula A and B: 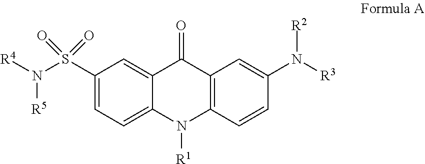
wherein
R¹, R², R³, R⁴, R⁵ are independent from each other and may represent:
H, CH₃, C₂H₅, a straight or branched C₃-C₁₂, preferably C₃-C₆, alkyl or perfluoroalkyl group, a phosphonylated alkyl group (CH₂)ₘP(O)(OH)₂, where m=1-12, preferably 2-6, with a straight or branched alkyl chain, (CH₂)ₙCOOH, where n=1-12, preferably 1-5, or (CH₂)ₙCOOR⁶, where n=1-12, preferably 1-5, and
R⁶ may be alkyl, in particular C₁-C₆, CH₂CN, benzyl, fluorene-9-yl, polyhalogenoalkyl, polyhalogenophenyl, e.g. tetra- or pentafluorophenyl, pentachlorophenyl, 2- and 4-nitrophenyl, N-succinimidyl, sulfo-N-succinimidyl, 1-oxybenzotriazolyl, or other potentially nucleophile-reactive leaving groups, alkyl sulfonate ((CH₂)ₙSO₃H) or alkyl sulfate ((CH₂)ₙOSO₃H) where n=1-12, preferably 1-5, and the alkyl chain in any (CH₂)ₙ may be straight or branched;
a hydroxyalkyl group (CH₂)ₘOH or thioalkyl group (CH₂)ₘSH, where m=1-12, preferably 2-6, with a straight or branched alkyl chain, a phosphorylated hydroxyalkyl group (CH₂)ₘOP(O)(OH)₂, where m=1-12, preferably 2-6, with a straight or branched alkyl chain; one of R¹ or R² groups may be a carbonate or carbamate derivative of (CH₂)ₘOCOOR⁷ or COOR⁷, where m=1-12 and R⁷=methyl, ethyl, tert-butyl, benzyl, fluoren-9-yl, CH₂CN, N-succinimidyl, sulfo-N-succinimidyl, 1-oxybenzotriazolyl, phenyl, substituted phenyl group, e.g., 2- or 4-nitrophenyl, pentachlorophenyl, penta-fluorophenyl, 2,3,5,6-tetrafluorophenyl, 2-pyridyl, 4-pyridyl, pyrimid-4-yl;
(CH₂)ₘNR^{a}R^{b}, where m=1-12, preferably 2-6, with a straight or branched alkyl chain; R^{a}, R^{b} are independent from each other and represent hydrogen and/or C₁-C₄ alkyl groups, a hydroxyalkyl group (CH₂)ₘOH, where m=2-6, with a straight or branched alkyl chain, a phosphorylated hydroxyalkyl group
(CH₂)ₘOP(O)(OH)₂, where m=1-12, preferably 2-6, with a straight or branched alkyl chain;
an alkyl azide (CH₂)ₘN₃, where m=m=1-12, preferably 2-6, with a straight or branched alkyl chain;
R¹, R², R³, R⁴, R⁵ may contain a terminal alkyloxyamino group (CH₂)ₘONH₂, where m=1-12, preferably 2-6, with a straight or branched alkyl chain, that can include one or multiple alkylamino (CH₂)ₘNH or alkylamido (CH₂)ₘCONH groups in all possible combinations with m=0-12;
(CH₂)ₙCONHR^{B}, with n=1-12, preferably 1-5; R⁸=H, C₁-C₆ alkyl, (CH₂)ₘN₃, or (CH₂)ₘ—N-maleimido, (CH₂)ₘ—NH—COCH₂X (X=Br or I), with m=1-12, preferably 2-6, and with straight or branched alkyl chains in (CH₂)ₙ, (CH₂)ₘ and R⁸;
a primary amino group, preferably as R¹, R², or R³, which forms aryl hydrazines;
a hydroxy group, preferably as R² or R³, which forms aryl hydroxylamines;
further, one of the residues R¹, R², R³, R⁴, R⁵ may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl;
additionally, R²-R³ (R⁴-R⁵) may form a four-, five, six-, or seven-membered cycle, or a four-, five, six-, or seven-membered cycle with or without a primary amino group NH₂, secondary amino group NHR^{a}, where R^{a}=C₁-C₆ alkyl, a hydroxyl group OH, or a phosphorylated hydroxyl group —OP(O)(OH)₂ attached to one of the carbon atoms in this cycle;
optionally R²-R³ (R⁴-R⁵) may form a four-, five, six-, or seven-membered heterocycle with an additional 1-3 heteroatoms such as O, N or S included into this heterocycle;
further, R¹ may represent an unsubstituted phenyl group, a phenyl group with one or several electron-donor substituents chosen from the set of OH, SH, NH₂, NHR^{a}, NR^{a}R^{b}, R^{a}O, R^{a}S, where R^{a} and R^{b} are independent from each other and may be C₁-C₆ alkyl groups with straight or branched carbon chains, a phenyl group with one or several electron-acceptors chosen from the set of NO₂, CN, COH, COOH, CH═CHCN, CH═C(CN)₂, SO₂R^{a}, COR^{a}, COOR^{a}, CH═CHCOR^{a}, CH═CHCOOR^{a}, CONHR^{a}, SO₂NR^{a}R^{b}, CONR^{a}R^{b}, where R^{a} and R^{b} are independent from each other and may be H, or C₁-C₆ alkyl group(s) with straight or branched carbon chains;
or R¹ may represent a heteroaromatic group;
with the proviso that in all compounds of Formula A above at least two, preferably at least 3, 4, 5 or 6 negatively charged groups are present under basic conditions, i.e. 7<pH<14, and these negatively charged groups represent at least partially deprotonated residues of ionizable groups selected from the following:
SH, COOH, a sulfonic acid residue SO₃H, a primary phosphate group OP(O)(OH)₂, a secondary phosphate group OP(O)(OH)R^{a}, where R^{a}=C₁-C₄ alkyl or substituted C₁-C₄ alkyl, a primary phosphonate group P(O)(OH)₂, a secondary phosphonate group P(O)(OH)R^{a}, where R^{a}=C₁-C₄ alkyl or substituted C₁-C₄ alkyl;
and compounds of Formula A can exist and can be used as salts, solvates and hydrates, preferably as salts with alkaline metal cations including Na⁺, Li⁺, K⁺ and organic ammonium or organic phosphonium cations; 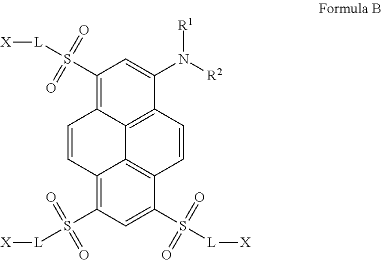
wherein R¹ and/or R² are independent from each other and may represent:
H, CH₃, C₂H₅, a linear or branched C₃-C₁₂ alkyl or perfluoroalkyl group, or a substituted C₂-C₆₁₂ alkyl group; in particular, (CH₂)ₙCOOR³, where n=1-12, preferably 1-5, R³ may be H, alkyl, in particular C₁-C₆, CH₂CN, benzyl, fluorene-9-yl, polyhalogenoalkyl, polyhalogenophenyl, e.g. tetra- or pentafluorophenyl, pentachlorophenyl, 2- and 4-nitrophenyl, N-succinimidyl, sulfo-N-succinimidyl, 1-oxybenzotriazolyl, or other potentially nucleophile-reactive leaving groups, and the alkyl chain in (CH₂)ₙ may be straight or branched; and
R¹-R² may form a four-, five, six-, or seven-membered non-aromatic carbocycle with an additional primary amino group NH₂, secondary amino group NHR^{a}, where R^{a}=C₁-C₆ alkyl, or hydroxyl group OH attached to one of the carbon atoms in this cycle; optionally R¹-R² may form a four-, five, six-, or seven-membered non-aromatic heterocycle with an additional heteroatom such as O, N or S included into this heterocycle;
a hydroxyalkyl group (CH₂)ₘOH, where m=1-12, preferably 2-6, with a straight or branched alkyl chain; one of R¹ or R² groups may be a carbonate or carbamate derivative (CH₂)ₘOCOOR⁴ or COOR⁴, where m=1-12 and R⁴=methyl, ethyl, 2-chloroethyl, N-succinimidyl, sulfo-N-succinimidyl, 1-oxybenzotriazolyl, a phenyl group or substituted phenyl group, e.g., 2- and 4-nitrophenyl, pentachlorophenyl, pentafluorophenyl, 2,3,5,6-tetrafluoro-phenyl, 2-pyridyl, or 4-pyridyl;
(CH₂)ₘNR^{a}R^{b}, where m=1-12, preferably 2-6, with a straight or branched alkyl chain; R^{a}, R^{b} are independent from each other and may be H, or optionally substituted C₁-C₄ alkyl group(s), in particular, one of R¹ or R² groups may be an alkyl azide group (CH₂)ₘN₃ with m=2-6 and a straight or branched alkyl chain;
one of R¹ or R² may be (CH₂)ₙSO₂NR⁵NH₂ with n=1-12, while the substituent R⁵ can be represented by H, alkyl, hydroxyalkyl or perfluoroalkyl groups C₁-C₁₂;
one of R¹ or R² groups may be a primary amino group to form aryl hydrazines Ar—NR⁶NH₂ where Ar is the entire pyrene residue in Formula B and R⁶=H or alkyl;
one of R¹ or R² groups may be a hydroxy group to form aryl hydroxylamines Ar—NR⁷OH where Ar is the entire pyrene residue in Formula B and R⁷=H or alkyl;
one of R¹ or R² groups may contain a terminal alkyloxyamino group (CH₂)ₙONH₂ with n=1-12, which can be linked via one or multiple alkylamino (CH₂)ₘNH, alkylamido (CH₂)ₘCONH, alkyl ether or ester group(s) in all possible combinations with m=0-12;
one of R¹ or R² groups may be CO(CH₂)ₙCOOR^{B}, with n=1-5 and a straight or branched alkyl chain (CH₂)ₙ and with R⁸ selected from H, straight or branched C₁-C₆ alkyl, CH₂CN, 2- and 4-nitrophenyl, 2,3,5,6-tetrafluorophenyl, pentachlorophenyl, pentafluoro-phenyl, N-succinimidyl, sulfo-N-succinimidyl, 1-oxybenzotriazolyl;
further, one of R¹ or R² may be (CH₂)ₙCONHR⁹, with n=1-5 and R⁹=H, C₁-C₆ alkyl, (CH₂)ₘN₃, (CH₂)ₘ—N-maleimido, (CH₂)ₘ—NHCOCH₂X (X=Br or I), where m=2-6 and with straight or branched alkyl chains in (CH₂)ₙ and R⁹;
or one of R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl; or one of R¹ or R² may be an alkyl azide (CH)N₃ or alkine, in particular propargyl;
the linker L comprises at least one carbon atom and may comprise alkyl, heteroalkyl, in particular alkyloxy such as CH₂OCH₂, CH₂CH₂O CH₂CH₂OCH₂, alkylamino or dialkylamino, particularly diethanolamine or N-methyl (alkyl) monoethanolamine moieties such as N(CH₃)CH₂CH₂O— and N(CH₂CH₂O—)₂, perfluoroalkyl, like single or multiple difluoromethyl (CF₂), alkene or alkyne moieties in any combinations, at any occurrence, linear or branched, with the length ranging from C₁ to C₁₂;
the linker L may also include a carbonyl (CH₂CO, CF₂CO) moiety, also as part of an amide group;
the linker L may also comprise or contain a residue of 1,3,5-triazine, thus providing two attachment points for group X;
X denotes a solubilizing and/or ionizable anion-providing moiety, in particular consisting of or including a moiety selected from the group comprising hydroxyalkyl (CH₂)ₙOH, thioalkyl ((CH₂)ₙSH), carboxy alkyl ((CH₂)ₙCO₂H), alkyl sulfonate ((CH₂)ₙSO₃H), alkyl sulfate ((CH₂)ₙOSO₃H), alkyl phosphate ((CH₂)ₙOP(O)(OH)₂) or phosphonate ((CH₂)ₙP(O)(OH)₂), wherein n is an integer ranging from 0 to 12, or an analogon thereof wherein one or more of the CH₂ groups are replaced by CF₂,
further, the anion-providing moieties may be linked by means of non-aromatic O, N and S-containing heterocycles, e. g., piperazines, pipecolines, or, alternatively, one of the groups X may bear any of the moieties listed above for groups R¹ and R², also with any type of linkage listed for group L, and independently from other substituents;
Compounds of Formula B can exist and can be used as salts, solvates and hydrates, preferably as salts with alkaline metal cations including Na⁺, Li⁺, K⁺ and organic ammonium.
With the proviso that in all compounds represented by Formula B three or six negatively charged groups are present in the residues X of Formula B under basic conditions, i.e. 7<pH<14, and these negatively charged groups represent at least partially deprotonated residues of ionizable groups selected from the following:
SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl is provided;
and compounds of Formula B can exist and can be used as salts, solvates and hydrates, preferably as salts with alkaline metal cations including Na⁺, Li⁺, K⁺ and organic ammonium or organic phosphonium cations;

2. The method according to claim 1 wherein the standard of known composition is a standard base pair ladder and/or a known carbohydrate mixture composition.

3. A method for an automated carbohydrate mixture composition pattern profiling comprising the steps of
a) providing a first sample containing a first carbohydrate mixture composition;
b) labelling of said carbohydrate mixture composition with a first fluorescent label;
c) providing a second sample containing a second carbohydrate mixture composition labelled with a second fluorescent label which may be added optionally to said first sample;
d) generating electropherograms/chromatograms of the carbohydrate mixture composition of said sample using electrokinetic/chromatographic separation techniques combined with fluorescence or laser induced fluorescence detection;
e) analyzing the identity and/or differences between the carbohydrate mixture composition pattern profiles of the first and the second sample, wherein the first fluorescent label of the first sample is different to the second fluorescent label of the second sample and wherein at least one of the first fluorescent label and the second fluorescent label is a fluorescent dye as defined in claim 1.

4. A method for an automated carbohydrate mixture composition pattern profiling according to claim 3 comprising the steps of
a) providing a sample containing a first carbohydrate mixture composition;
b) labelling of said carbohydrate mixture composition with a first fluorescent label;
c) providing a second sample labelled with a second fluorescent label containing a second carbohydrate mixture composition to be compared with;
d) generating electropherograms/chromatograms of the carbohydrate mixture composition of the first and second sample using electrokinetic/chromatographic separation techniques combined with fluorescence or laser induced fluorescence detection;
e) comparing the standard migration/retention time indices calculated from the obtained electropherogram/chromatogram of the first sample and the second sample;
f) analyzing the identify and/or differences between the carbohydrate mixture composition pattern profiles of the first and second sample, wherein standard migration/retention time indices of the carbohydrates present in the sample are calculated based on internal standards of known composition labelled with a third fluorescent label and wherein one of the first or the second fluorescent label is a fluorescent dye as defined in claim 1.

5. The method according to claim 1 whereby at least two orthogonal standards are added to the sample and orthogonal cross-alignment is performed based on the given standard migration/retention time indices of the at least two orthogonal standards.

6. The method according to claim 1 wherein the sample contains a mixture of carbohydrates.

7. The method according to claim 1 wherein the sample is an extraction of glycans and the method allows for the identification of a glycosylation pattern profile.

8. The method according to claim 1 wherein the glycosylation pattern of a glycoprotein is identified.

9. The method according to claim wherein the components of the carbohydrate mixture are determined quantitatively.

10. A method for calibration of a multi wavelength fluorescence detection system, in particular, a capillary-gel electrophoresis system, with acridone and/or pyrene based fluorescent dyes which may optionally be present as conjugates with a substrate moiety including carbohydrates,
whereby the method includes the detection of at least one of the compounds according to Formula A or B as defined in claim 1 together with additional fluorescent dyes and their carbohydrate conjugates emitting at different wavelengths, preferably including at least one of the compounds: APTS, 6-R, 8-H, 15, 19, 20, 23 or 23b, as shown in the following scheme:

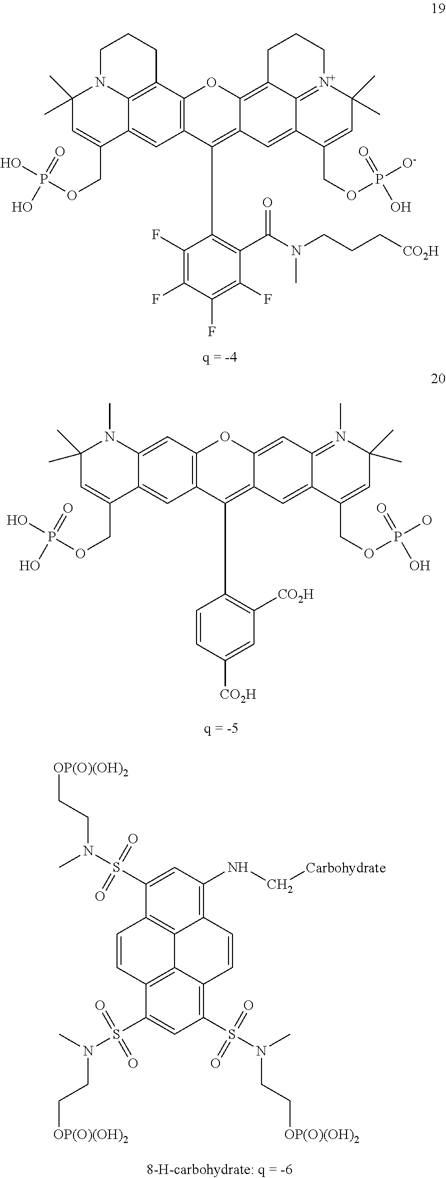

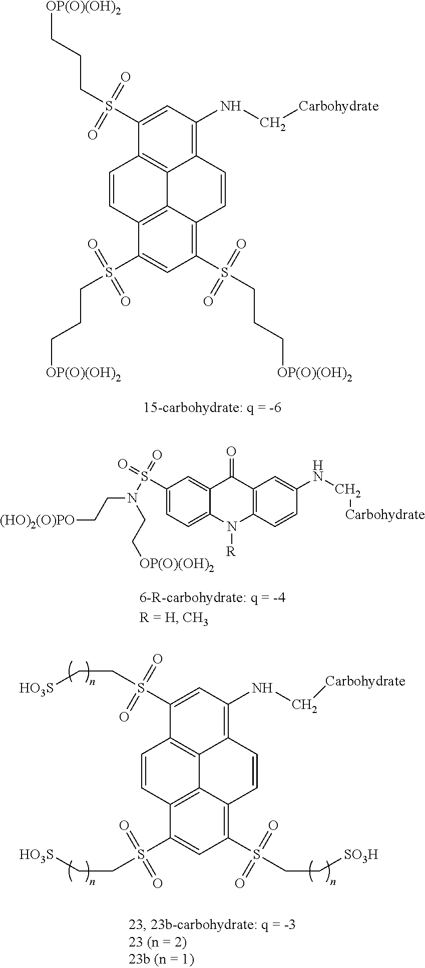

11. The method according to claim 10 wherein the acridone and/or pyrene based dyes, which may optionally be present as conjugates with a substrate moiety including carbohydrates, include the combination of APTS, 6-H, 19 and 20, or APTS, 6-Me, 19 and 20, or 15, 6-Me, 19 and 20, or APTS, 15, 19 and 20, or APTS, 15, 6-Me and 20, or APTS, 8-H, 6-Me and 19, or APTS, 8-H, 6-Me and 20, or APTS, 8-H, 19 and 20, or APTS, 23, 19 and 20, or APTS, 15, 6-Me and 19, or APTS, 23, 6-Me and 19, or APTS, 23, 6-Me and 20, or 23, 6-Me, 19 and 20, or APTS, 8-H, 6-Me, 20 and 19, or APTS, 15, 6-Me, 20 and 19, or APTS, 23, 6-Me, 20 and 19, or APTS, 8-H, 6-H, 20 and 19, or APTS, 15, 6-H, 20 and 19, or APTS, 23, 6-H, 20 and 19.

12. The method according to claim 1 wherein the fluorescent dye of Formula B is a dye having the following Formula C with n=0-12 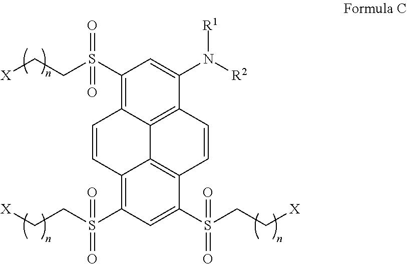
wherein
R¹ and/or R² are independent from each other and may represent:
H, CH₃, C₂H₅, a straight or branched C₃-C₁₂, preferably C₃-C₆, alkyl group, or a substituted C₂-C₁₂, preferably C₂-C₆, alkyl group; in particular, (CH₂)ₙCOOR³, where n=1-12, preferably 1-5, R³ may be H, CH₂CN, 2- and 4-nitrophenyl, 2,3,5,6-tetrafluorophenyl, pentachlorophenyl, pentafluorophenyl, N-succinimidyl, sulfo-N-succinimidyl, 1-oxybenzotriazolyl and the alkyl chain in (CH₂)ₙ may be straight or branched; and
R¹-R² may form a four-, five, six-, or seven-membered non-aromatic carbocycle with an additional primary amino group NH₂, secondary amino group NHR^{a}, where R^{a}=C₁-C₆ alkyl, or hydroxyl group OH attached to one of the carbon atoms in this cycle; optionally R¹-R² may form a four-, five, six-, or seven-membered non-aromatic heterocycle with an additional heteroatom such as 0, N or S included into this heterocycle;
a hydroxyalkyl group (CH₂)ₘOH, where m=1-12, preferably 2-6, with a straight or branched alkyl chain; one of R¹ or R² groups may be a carbonate or carbamate derivative (CH₂)ₘOCOOR⁴ or COOR⁴, where m=1-12 and R⁴=methyl, ethyl, 2-chloroethyl, N-succinimidyl, sulfo-N-succinimidyl, 1-oxybenzotriazolyl a phenyl group or substituted phenyl group, e.g., 2- and 4-nitrophenyl, pentachlorophenyl, pentafluorophenyl, 2,3,5,6-tetrafluoro-phenyl, 2-pyridyl, or 4-pyridyl;
(CH₂)ₘNR^{a}R^{b}, where m=1-12, preferably 2-6, with a straight or branched alkyl chain; R^{a}, R^{b} are independent from each other and may be H, or optionally substituted C₁-C₄ alkyl group(s), in particular, one of R¹ or R² groups may be an alkyl azide group (CH₂)ₘN₃ with m=2-6 and a straight or branched alkyl chain;
one of R¹ or R² groups may be (CH₂)ₙCOOR⁵, with n=1-5 and a straight or branched alkyl chain (CH₂)ₙ and with R⁵ selected from H, straight or branched C₁-C₆ alkyl, CH₂CN, 2- and 4-nitrophenyl, 2,3,5,6-tetrafluorophenyl, pentachlorophenyl, pentafluoro-phenyl, sulfo-N-succinimidyl, N-succinimidyl or 1-oxybenzotriazolyl;
further, one of R¹ or R² may be (CH₂)_{b}CONHR⁶, with n=1-12, preferably 1-5, and R⁶=H, C₁-C₆ alkyl, (CH₂)ₘN₃, (CH₂)ₘ—N-maleimido, (CH₂)ₘ—NHCOCH₂X (X=Br or I), where m=2-6 and with straight or branched alkyl chains in (CH₂)ₙ and R⁶; or one of R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl;
one of R¹ or R² groups may be a primary amino group to form aryl hydrazines Ar—NR⁶NH₂ where Ar is the entire pyrene residue in Formula C and R⁷=H or alkyl;
one of R¹ or R² groups may be a hydroxy group to form aryl hydroxylamines Ar—NR⁸OH where Ar is the entire pyrene residue in Formula C and R⁷=H or alkyl;
one of R¹ or R² groups may contain a terminal alkyloxyamino group (CH₂)ₙONH₂ with n=1-12, which can be linked via one or multiple alkylamino (CH₂)ₘNH, alkylamido (CH₂)ₘCONH, alkyl ether or alkyl ester group(s) in all possible combinations with m=0-12;
the (CH₂)ₙ—CH₂ linker, with n=1-5, between the SO₂ fragment and the residue X in Formula B may represent a straight-chain, branched or cyclic group having 2-6 carbon atoms;
X=SH, COOH, SO₃H, OP(O)(OH)₂, OP(O)(OH)R^{a}, where R^{a}=optionally substituted C₁-C₄ alkyl, P(O)(OH)₂, P(O)(OH)R^{a}, where R^{a}=optionally substituted C₁-C₄ alkyl;
with the proviso that in all compounds represented by Formula C three or six negatively charged groups are present in the residues X of Formula B under basic conditions, i.e. 7<pH<14, and these negatively charged groups represent at least partially deprotonated residues of ionizable groups selected from the following:
SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl;
and compounds of Formula C can exist and can be used as salts, solvates and hydrates, preferably as salts with alkaline metal cations including Na⁺, Li⁺, K⁺ and organic ammonium or organic phosphonium cations.

13. The method according to claim 1 wherein the fluorescent dye of Formula B is a dye having the following Formula D 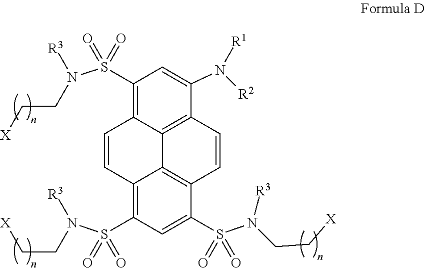
wherein
R¹ and/or R² are independent from each other and may represent H, CH₃, C₂H₅, or a straight or branched, optionally substituted, C₃-C₁₂, preferably C₃-C₆, alkyl group; in particular, (CH₂)ₙCOOR⁴, where n=1-12, preferably 1-5, R⁴ may be H, CH₂CN, 2- and 4-nitrophenyl, 2,3,5,6-tetrafluorophenyl, pentachlorophenyl, pentafluorophenyl, N-succinimidyl, sulfo-N-succinimidyl, 1-oxybenzotriazolyl and the alkyl chain in (CH₂)ₙ may be straight or branched; and
R¹-R² may form a four-, five, six-, or seven-membered non-aromatic carbocycle with an additional primary amino group NH₂, secondary amino group NHR^{a}, where R^{a}=optionally substituted C₁-C₆ alkyl, or hydroxyl group OH attached to one of the carbon atoms in this cycle; or optionally R¹-R² may form a four-, five, six-, or seven-membered non-aromatic heterocycle with a heteroatom such as 0, N or S included into this heterocycle;
R¹ and/or R² may further represent:
a hydroxyalkyl group (CH₂)ₘOH, where m=1-12, preferably 2-6, with a straight or branched, optionally substituted alkyl chain; one of R¹ or R² groups may be a carbonate or carbamate derivative (CH₂)ₘOCOOR⁵ or COOR⁵, where m=1-12 and R⁵=methyl, ethyl, 2-chloroethyl, CH₂CN, N-succinimidyl, sulfo-N-succinimidyl, 1-oxybenzotriazolyl, a phenyl group or substituted phenyl group, such as 2- and 4-nitrophenyl, pentachlorophenyl, pentafluoro-phenyl, 2,3,5,6-tetrafluorophenyl, 2-pyridyl, 4-pyridyl;
(CH₂)ₘN₃, m=1-12, preferably 2-6, with a straight or branched alkyl chain;
(CH₂)ₙCONHR⁶, where n=1-12, preferably 1-5 and R⁶=H, substituted or unsubstituted C₁-C₆ alkyl, (CH₂)ₘN₃, (CH₂)ₘ—N-maleimido, (CH₂)m-NHCOCH₂Y (Y=Br, I) where m=1-12, preferably 2-6, with straight or branched alkyl chains in (CH₂)ₙ and R⁶;
one of R¹ or R² groups may be a primary amino group to form aryl hydrazines Ar—NR⁷NH₂ where Ar is the entire pyrene residue in Formula D and R⁷=H or alkyl;
one of R¹ or R² groups may be a hydroxy group to form aryl hydroxylamines Ar—NR⁸OH where Ar is the entire pyrene residue in Formula D and R⁸=H or alkyl;
one of R¹ or R² groups may contain a terminal alkyloxyamino group (CH₂)ₙONH₂ with n=1-12, which can be linked via one or multiple alkylamino (CH₂)ₘNH, alkylamido (CH₂)ₘCONH, alkyl ether or alkyl ester group(s) in all possible combinations with m=0-12;
further, R¹ or R² may represent CH₂—C₆H₄—NH₂, COC₆H₄—NH₂, CONHC₆H₄—NH₂ or CSNHC₆H₄—NH₂ with C₆H₄ being a 1,2-, 1,3- or 1,4-phenylene, COC₅H₃N—NH₂ or CH₂—C₅H₃N—NH₂, with C₅H₃N being pyridin-2,4-diyl, pyridin-2,5-diyl, pyridin-2,6-diyl, or pyridin-3,5-diyl;
R³=H, (CH₂)_{q}CH₂X, C₂H₅, a straight or branched C₃-C₆ alkyl group, CₘH₂ₘOR, where m=2-6, with a straight or branched alkan-diyl chain CₘH₂ₘ, and R=H, CH₃, C₂H₅, C₃H₇, CH₃(CH₂CH₂O)ₖCH₂CH₂; with k=1-12; while the (CH₂)_{q}CH₂ linker may represent a straight-chain, branched or cyclic group having 2-6 carbon atoms;
in Formula D, the (CH₂)ₙ—CH₂ linker, with n=1-12, preferably 1-5, between the sulfonamide fragment SO₂N and the residue X may represent a straight-chain, branched or cyclic group having 2-6 carbon atoms;
X=SH, COOH, SO₃H, OP(O)(OH)₂, OP(O)(OH)R^{a}, where R^{a}=substituted or unsubstituted C₁-C₄ alkyl, P(O)(OH)₂, P(O)(OH)R^{a}, where R^{a}=substituted or unsubstituted C₁-C₄ alkyl;
with the proviso that in all compounds represented by Formula D three, six, nine or twelve negatively charged groups are present in the residues X of Formula C under basic conditions, i.e. 7<pH<14, and these negatively charged groups represent at least partially deprotonated residues of ionizable groups selected from the following: SH, COOH, SO₃H, OP(O)(OH)₂, OP(O)(OH)R^{a}, where R^{a}=C₁-C₄ alkyl or substituted C₁-C₄ alkyl, P(O)(OH)₂, P(O)(OH)R^{a}, where R^{a}=C₁-C₄ alkyl or substituted C₁-C₄ alkyl;
and compounds of Formula D can exist and can be used as salts, solvates and hydrates, preferably as salts with alkaline metal cations including Na⁺, Li⁺, K⁺ and organic ammonium or organic phosphonium cations.

14. The method according to claim 1 wherein R¹ and/or R² in formula B, or D represent:
H, deuterium, alkyl or deutero-substituted substituted alkyl, wherein one, several or all H atoms of the alkyl group may be replaced by deuterium atoms, in particular alkyl or deutero-alkyl with 1-12 C atoms, preferably 1-6 C atoms, 4,6-dihalo-1,3,5-triazinyl (C₃N₃X₂) where halogen X is preferably chlorine, 2-, 3- or 4-aminobenzoyl (COC₆H₄NH₂), N-[(2-, N-[(3- or N-[(4-aminophenyl)ureido group (NHCONHC₆H₄NH₂), N-[(2-, N-[(3- or N-[(4-aminophenyl)thioureido group (NHCSNHC₆H₄NH₂ or linked carboxylic acid residues and their reactive esters of the general formulae (CH₂)ₘ₁COOR³, (CH₂)ₘ₁OCOOR³ (CH₂)ₙ₁COOR³ or (CO)ₘ₁(CH₂)ₘ₂(CO)ₙ₁(NH)ₙ₂(CO)ₙ₃(CH₂)ₙ₄COOR³ where the integers m1, m2 and n1, n2, n3, n4 independently range from 1 to 12 and from 0 to 12, respectively, with the chain (CH₂)_{m/n} being straight, branched, saturated, unsaturated, partially or completely deuterated, and/or or included into a carbo- or heterocylcle containing N, O or S, whereas R³ is H, D or a nucleophile-reactive leaving group, preferably including but not limited to N-succinimidyl, sulfo-N-succinimidyl, 1-oxybenzotriazolyl, cyanomethyl, polyhalogenoalkyl, polyhalogenophenyl, e.g. tetra- or pentafluorophenyl, 2- or 4-nitrophenyl.

15. The method according to claim 1 wherein the compound of Formulae A to B is selected from: 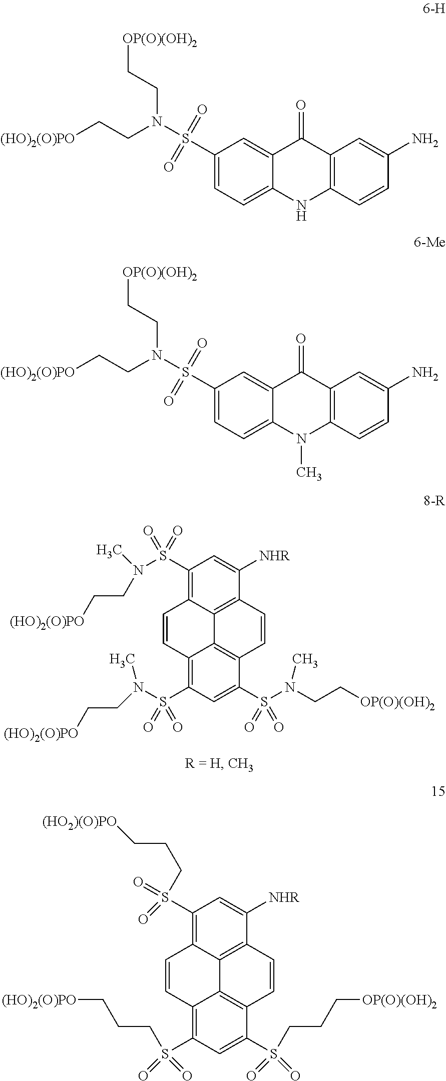
or a compound of 7-R (R=H, Me), 13a, 13b, 16, 18, 23 and 23b 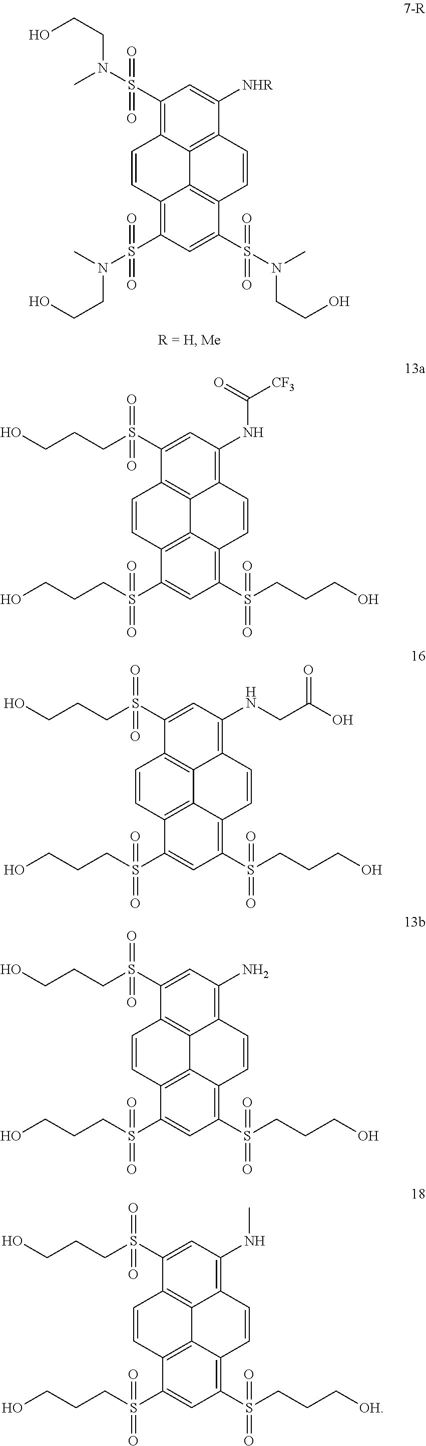 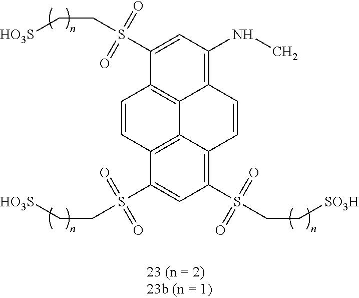
or salts thereof.

16. A kit or system for determining and/or identifying carbohydrate mixture composition patterns comprising a data processing unit having a non-transient memory, said memory containing a database, said database containing aligned migration/retention times and/or aligned migration/retention time indices of carbohydrates, said migration/retention times and/or migration/retention time indices are obtained by an automated determination and/or identification of carbohydrates and/or identification of carbohydrates and/or carbohydrate mixture composition pattern profiling comprising the steps of:
a) obtaining a sample containing at least one carbohydrate;
b) labelling said carbohydrate(s) with a first fluorescent label;
c) providing a standard of known composition labelled with a second fluorescent label;
d) determining the migration/retention time(s) of said carbohydrate(s) and the standard of known composition using electrokinetic/chromatographic separation techniques combined with fluorescence or laser induced fluorescence detection;
e) aligning the migration/retention time(s) to migration/retention time indice(s) based on given standard migration/retention time indice(s) of the standard;
f) comparing these migration/retention time indice(s) of the carbohydrate(s) with standard migration/retention time indice(s) from a database;
g) identifying or determining the carbohydrate(s) and/or the carbohydrate mixture composition pattern,
wherein the standard composition is added to the sample containing the unknown carbohydrate mixture composition, the first fluorescent label and the second fluorescent label are different and wherein the first fluorescent label or the second fluorescent label is a fluorescent dye, preferably having multiple ionizable and/or negatively charged groups which is selected from the group consisting of compounds of the general Formulae A to B and a fluorescent dye as defined in claim 1.

17. A kit or system for an automated carbohydrate mixture composition pattern profiling comprising a data processing unit having a non-transient memory, said memory containing a database, said database containing aligned migration/retention times and/or aligned migration/retention time indices of carbohydrates, said migration/retention times and/or migration/retention time indices are obtained by an automated determination and/or identification of carbohydrates and/or identification of carbohydrates and/or carbohydrate mixture composition pattern profiling comprising the steps of
a) providing a first sample containing an unknown carbohydrate mixture composition;
b) labelling of said carbohydrate mixture composition with a first fluorescent label;
c) adding a second sample having a known carbohydrate mixture composition pattern labelled with a second fluorescent label to said first sample;
d) generating electropherograms/chromatograms of the carbohydrate mixture composition of said sample using electrokinetic/chromatographic separation techniques combined with fluorescence or laser induced fluorescence detection, like capillary gel electrophoresis-laser induced fluorescence;
e) analyzing the identity and/or differences between the carbohydrate mixture composition pattern profiles of the first and the second sample, wherein the first fluorescent label of the first sample is different to the second fluorescent label of the second sample and wherein at least one of the first fluorescent label and the second fluorescent label is a fluorescent dye as defined in claim 1.

18. A kit or system according to claim 16 further comprising a capillary gel electrophoresis-laser induced fluorescence apparatus, in particular, wherein the capillary gel electrophoresis-laser induced fluorescence apparatus is a capillary DNA-sequencer.

19. A carbohydrate dye conjugate comprising fluorescent dyes as defined in and used in the method of claim 1.

20. The carbohydrate dye conjugate according to claim 19 wherein the dye is selected from the compounds of the formula below

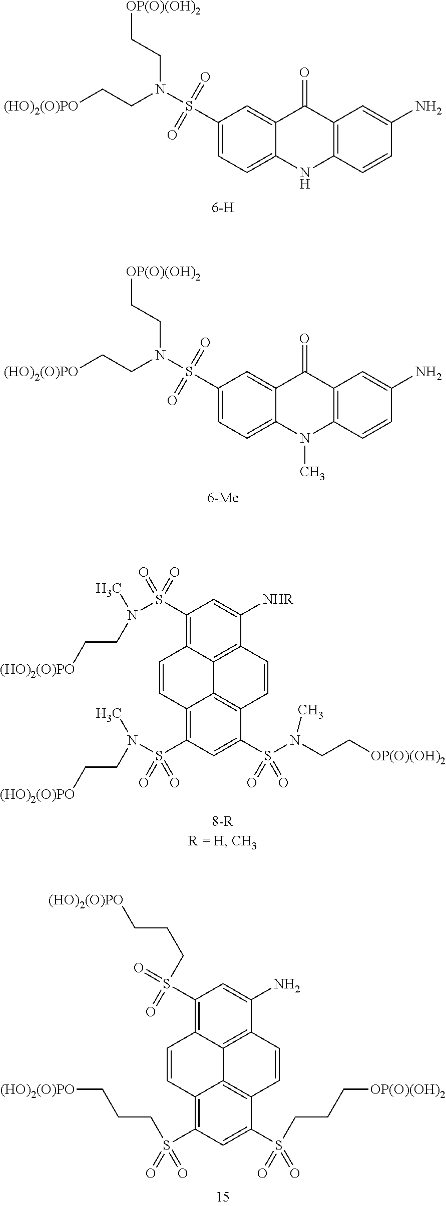

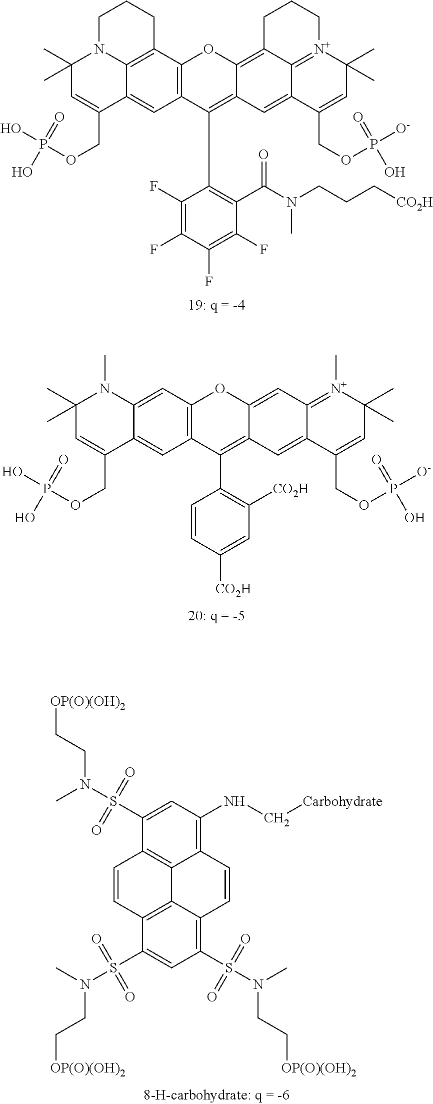

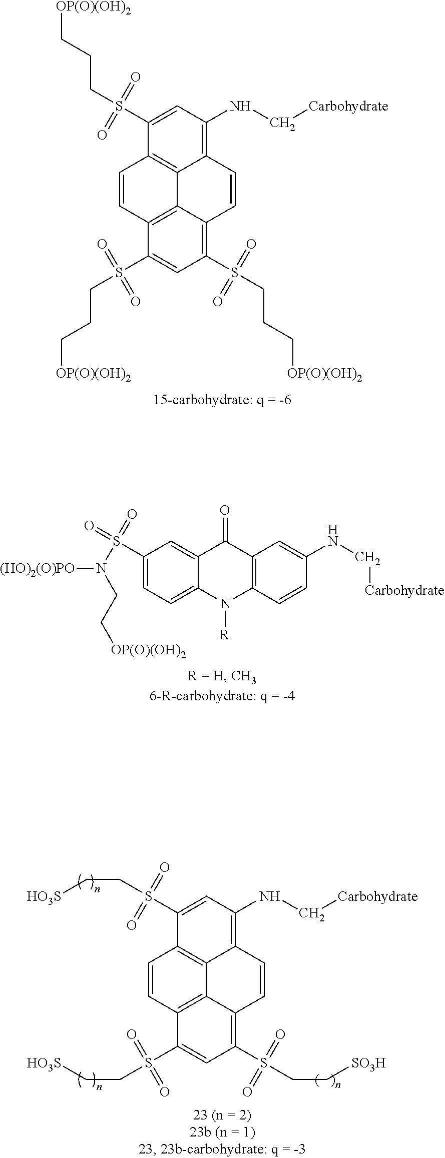

21. A kit or composition comprising one or more of the dyes as defined in and for use in the method of claim 1.

22. A calibration standard, like an oligosaccharide standard, including a fluorescence dye according to Formula A, B, C or D which may be conjugated with a carbohydrate, optionally further comprising at least one of compounds 19, 20.

23. A kit containing a calibration standard according to claim 22 and, optionally, instructions for use.

24. A compound having the Formula 20

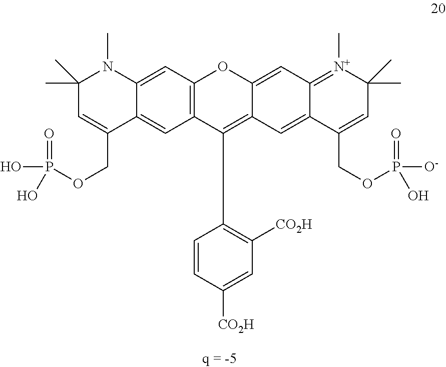 [SMILES: CN1c2cc3c(cc2C(COP(=O)(O)O)=CC1(C)C)C(c1ccc(C(=O)O)cc1C(=O)O)=c1cc2c(cc1O3)=[N+](C)C(C)(C)C=C2COP(=O)([O-])O]

25. A standard composition composed of compounds labelled with a fluorescence dye according to Formula A or B, in particular, of Formula C or D or different dyes of Formulae A to D.

26. The standard composition according to claim 25 being composed of carbohydrates labelled with a fluorescence dye according to Formula A or B, in particular, of Formula C or D or different dyes of Formulae A to D.

27. The standard composition according to claim 25 wherein the fluorescence dye is at least one dye selected from 6-H, 6-Me, 8-R, 15, 13a, 13b, 16, 18, 23 and 23b.

28. (canceled)

29. A kit or composition comprising one or more of the carbohydrate dye conjugates of claim 19.
